(19) United States
(12) Patent Application Publication
(10) Pub. No.: US 2019/0015423 A1
(43) Pub. Date: Jan. 17, 2019

(54) REGULATION OF GENE EXPRESSION BY MODULATING PRIMARY CILIA LENGTH

(71) Applicant: THE TRUSTEES OF COLUMBIA UNIVERSITY IN THE CITY OF NEW YORK, NEW YORK, NY (US)

(72) Inventors: Christopher Jacobs, New York, NY (US); Milos Spasic, New York, NY (US)

(21) Appl. No.: 16/144,779

(22) Filed: Sep. 27, 2018

Related U.S. Application Data
(63) Continuation-in-part of application No. PCT/US2017/025063, filed on Mar. 30, 2017.
(60) Provisional application No. 62/315,545, filed on Mar. 30, 2016.

(51) Int. Cl. A61K 31/55; A61K 31/165; A61K 31/135; A61K 31/485; A61P 19/10

(52) U.S. Cl. CPC A61K 31/55; A61K 31/165; A61K 31/135; A61K 45/06; A61P 19/10; A61K 2300/00; A61K 31/485

(57) ABSTRACT

The presently disclosed subject matter relates to methods of regulating gene expression in a cell by modulating the length of primary cilia of a cell, wherein such modulation can modulate the mechanosensitivity of the cell. The presently disclosed subject matter also provides for methods of treating ciliopathies and osteoporosis in a subject by increasing the length of primary cilia of a cell in a subject.

### CROSS-REFERENCE TO RELATED APPLICATION

This application is a continuation-in-part of International Patent Application No. PCT/US2017/025063 filed Mar. 30, 2017; which claims priority to U.S. Provisional Application Ser. No. 62/315,545 filed on Mar. 30, 2016, each of which are incorporated in its entirety herein.

### STATEMENT REGARDING FEDERALLY SPONSORED RESEARCH

This invention was made with government support under grant AR062177 awarded by the National Institutes of Health. The government has certain rights in the invention.

### FIELD OF INVENTION

The presently disclosed subject matter relates to methods of regulating gene expression in a cell by modulating the length of primary cilia of the cell. The presently disclosed subject matter also provides for methods of treating ciliopathies and osteoporosis.

### SEQUENCE LISTING

The specification further incorporates by reference the Sequence Listing submitted herewith via EFS on Mar. 30, 2017. Pursuant to 37 C.F.R. § 1.52(e)(5), the Sequence Listing text file, identified as seqlisting03292017.txt, is 656 bytes and was created on Mar. 29, 2017. The Sequence Listing, electronically filed herewith, does not extend beyond the scope of the specification and thus does not contain new matter.

### BACKGROUND

Mechanotransduction is a critical cellular process in a variety of tissues. Endothelial cells sense blood flow and transduce the mechanical stimuli into biochemical responses to adjust blood vessel diameter (Ku, 1997). Kidney epithelial cells in the collecting duct similarly sense and respond to varying rates of urine flow (Liu et al., 2003). Bone maintenance requires mechanical stimulation to maintain balanced formation and resorption (You et al., 2008). Understanding how cells sense mechanical cues and transduce them into biochemical responses is an important aspect of developing novel treatments for a wide variety of diseases of structural tissues.

Primary cilia are single immotile organelles extending from the surface of nearly all mammalian cells, and have been implicated as mechanosensors in a variety of cell types. It has been demonstrated that kidney epithelial cells respond to fluid flow, and specifically, that this mechanical stimulation causes primary cilia deflection (Praetorius and Spring, 2001). Furthermore, fluid flow initiates an intracellular calcium increase that is diminished when cilia are removed (Praetorius and Spring, 2003). Primary cilia have since been identified as mechanosensing organelles in a variety of cell types, including bone (Malone et al., 2007).

However, despite extensive efforts and the important medical implications of primary cilia function, the relation between cilium length, mechanosensitivity and gene expression has remained elusive.

### SUMMARY

The presently disclosed subject matter relates to methods of regulating gene expression in a cell by modulating the length of primary cilia of the cell. In certain embodiments, the method of regulating expression of a gene in a cell comprises administering to the cell (e.g., contacting the cell with) an effective amount of one or more cilium elongation modulator, wherein the cilium elongation modulator modulates a length of one or more primary cilia of the cell.

In certain embodiments, the one or more cilium elongation modulator is contacted to the cell in an amount effective to increase the length of one or more primary cilia of the cell.

In certain embodiments, the cilium elongation modulator regulates gene expression by modulating mechanosensitivity of the cell.

In certain embodiments, the one or more cilium elongation modulator is contacted to the cell in an amount effective to increase the mechanosensitivity of the cell.

In certain embodiments, the cilium elongation modulator modulates the length of one or more primary cilia of the cell by modulating the cAMP level.

In certain embodiments, the cilium elongation modulator modulates an expression level or an enzymatic activity of adenylyl cyclase, by which the cAMP level of the cell is modulated.

In certain embodiments, the cilium elongation modulator increases the length of one or more primary cilia of the cell by increasing the cAMP level.

In certain embodiments, the cilium elongation modulator increases an expression level or an enzymatic activity of adenylyl cyclase, by which the cAMP level of the cell is increased.

In certain embodiments, the cilium elongation modulator comprises one or more of fenoldopam, lithium, derivatives thereof, or combinations thereof.

In certain embodiments, the cilium elongation modulator comprises an adenylyl cyclase agonist. In certain embodiments, the cilium elongation modulator is selected from the group consisting of fenoldopam, forskolin, NKH 477 (CAS No: 138605-00-2), PACAP 1-27 (CAS No: 127317-03-7), PACAP 1-38 (137061-48-4), and combinations thereof.

In certain embodiments, the cilium elongation modulator comprises a dopamine D1-like receptor agonist. In certain embodiments, the cilium elongation modulator is selected from the group consisting of fenoldopam, Dihydrexidine (CAS No: 158704-02-0), Dopamine (CAS No: 62-31-7), NPEC-caged-dopamine (CAS No: 1257326-23-0), SKF 38393 (CAS No: 20012-10-6), SKF 77434 (CAS No: 300561-58-4), SKF 81297 (CAS No: 67287-39-2), SKF 82958 (CAS No: 74115-01-8), SKF 83822 (CAS No: 74115-10-9), SCH-23390 (CAS No: 87075-17-0), SKF-83959 (CAS No: 67287-95-0), A68930 (CAS No: 130465-39-3), A77636 (CAS No: 145307-34-2), (R)-(−)-Apomorphine (CAS No: 314-19-2), CY 208-243 (CAS No: 100999-26-6), Ecopipam (SCH-39,166, CAS No: 112108-01-7), and combinations thereof.

In certain embodiments, the cilium elongation modulator is lithium, derivatives thereof, or combinations thereof.

In certain embodiments, the cell being treated with the effective amount of a cilium elongation modulator is an osteocyte, an osteoblast, an osteoclast, an osteoprogenitor cell, or a combination thereof.

In certain embodiments, the gene expression modulated by contacting the cell with an effective amount of one or more cilium elongation modulator is an osteogenic gene.

In certain embodiments, the one or more cilium elongation modulator is contacted to the cell in an amount effective to increase expression of one or more gene, for example, an osteogenic gene. In certain embodiments, the cilium elongation modulator is contacted to the cell in an amount effective to increase a detectable level of expression of the one or more genes by at least about 10%, 20%, 30%, 40%, 50%, 60%, 70%, 80%, 90%, 95%, 99% or more compared to a cell not contacted with the cilium elongation modulator

In certain embodiments, the osteogenic gene is selected from the group consisting of COX-2, OPN, BSP, Collagen I, Osteocalcin, and combinations thereof.

In certain embodiments, the cilium elongation modulator is contacted to a population of cells in an amount effective to increase a detectable level of expression of said one or more genes in at least about 10%, 20%, 30%, 40%, 50%, 60%, 70%, 80%, 90%, 95%, 99% or more of the cells.

In certain embodiments, regulating expression of the osteogenic gene causes osteogenesis.

In certain embodiments, increasing the expression of the osteogenic gene increases osteogenesis.

In certain embodiments, the cell is a mammalian cell.

In certain embodiments, the mammalian cell is a human cell.

In certain embodiments, the cilium elongation modulator may be selected from a dihydrofolate reductase antagonist, an inhibitor of DNA synthesis or RNA synthesis, a glycogen synthase kinase 3 inhibitor, a topoisomerase inhibitor, a nucleoside analogue, an HDAC inhibitor, a 5-HT modulator, an anthelmintic, an epidermal growth factor receptor kinase inhibitor, a histamine receptor modulator, a dopamine D1 receptor agonist, a checkpoint kinase 1 inhibitor, a microtubule stabilizer, a reactive oxygen species modulator, a sodium channel blocker, an adrenoreceptor modulator, an inosine-5′-monophosphate dehydrogenase inhibitor, a benzodiazepine inverse agonist, an opioid receptor modulator, and an antiseptic.

In certain embodiments, the cilium elongation modulator may be selected from (S)-(+)-Niguldipine hydrochloride (CAS No.: 113145-69-0), 10-hydroxycamptothecin (CAS No.: 19685-09-7), 110-Phenanthroline monohydrate (CAS No.: 5144-89-8), 2 4-dihydroxychalcone 4-glucoside (CAS No.: 25515-43-9), 2-Methoxyestradiol (CAS No.: 362-07-2), 5-BDBD (CAS No.: 768404-03-1), 5-fluorouracil (CAS No.: 51-21-8), 62-dimethoxyflavone, 6-aminonicotinamide (CAS No.: 329-89-5), AG 494 (CAS No.: 139087-53-9), AM 630 (CAS No.: 164178-33-0), Amethopterin (R,S) (CAS No.: 60388-53-6), aminopterin (CAS No.: 54-62-6), BAY 61-3606 hydrochloride hydrate (CAS No.: 732938-37-8), Benzethonium chloride (CAS No.: 121-54-0), betahistine hydrochloride (CAS No.: 15430-48-5), beta-toxicarol (CAS No.: 82-11-1), BIO (CAS No.: 667463-62-9), Bromhexine HCl (CAS No.: 3572-43-8), Bufexamac (CAS No.: 2438-72-4), BW 373U86 (CAS No.: 155836-50-3), CP 339818 hydrochloride (CAS No.: 478341-55-8), CPT 11 (CAS No.: 100286-90-6), CY 208-243 (CAS No.: 100999-26-6), cycloheximide (CAS No.: 66-81-9), Cyclosporin A (CAS No.: 59865-13-3), deacetoxy-7-oxogedunin (CAS No.: CAS No. 13072-74-7), DEGUELIN(−) (CAS No.: 522-17-8), dehydrocholic acid (CAS No.: 81-23-2), DEOXYGEDUNIN (CAS No.: 21963-95-1), deoxykhivorin, deoxysappanone b 73-dimethyl ether acetate (CAS No.: 113122-54-6), dihydrocelastryl diacetate, Diphenyleneiodonium chloride (CAS No.: 4673-26-1), Docetaxel (CAS No.: 114977-28-5), Dorzolamide HCl (CAS No.: 130693-82-2), Droxinostat (CAS No.: 99873-43-5), duartin dimethyl ether, estradiol cypionate (CAS No.: 313-06-4), Ethacridine lactate monohydrate (CAS No.: 1837-57-6), Etonitazenyl isothiocyanate (CAS No.: 85951-65-1), Floxuridine (CAS No.: 50-91-9), Ginkgolide B (CAS No.: 15291-77-7), GW 1929 (CAS No.: 196808-24-9), harmalol hydrochloride (CAS No.: 6028-07-5), hecogenin acetate (CAS No.: 915-35-5), homidium bromide (CAS No.: 1239-45-8), hycanthone (CAS No.: 3105-97-3), IC 261 (CAS No.: 186611-52-9), Indirubin-3′-oxime (CAS No.: 160807-49-8), Irinotecan (CAS No.: 97682-44-5), itraconazole (CAS No.: 84625-61-6), Kenpaullone (CAS No.: 142273-20-9), khivorin (CAS No.: 2524-38-1), levulinic acid 3-benzylidenyl-, Lonidamine (CAS No.: 50264-69-2), Loratidine (CAS No.: 79794-75-5), Merbromin (CAS No.: 129-16-8), mercaptopurine (CAS No.: 50-44-2), Methotrexate (CAS No.: 59-05-2), methoxyamine hydrochloride (CAS No.: 67-62-9), monensin sodium (CAS No.: 22373-78-0), monobenzone (CAS No.: 103-16-2), Mycophenolate mofetil (CAS No.: 128794-94-5), Oxfendazole (CAS No.: 53716-50-0), pararosaniline pamoate (CAS No.: 7460-07-3), PD 169316 (CAS No.: 152121-53-4)), PD-407824 (CAS No.: 622864-54-4), Pemetrexed (CAS No.: 137281-23-3), perillic acid (−) (CAS No.: 23635-14-5), PHA 767491 hydrochloride (CAS No.: 942425-68-5), PIM 1 Inhibitor 2 (CAS No.: 477845-12-8), Piperlongumine (CAS No.: 20069-09-4), Pralatrexate (CAS No.: 146464-95-1), pyrimethamine (CAS No.: 58-14-0), pyrvinium pamoate (CAS No.: 3546-41-6), quinidine gluconate (CAS No.: 56-54-2), quinine sulfate (CAS No.: 6119-70-6), reserpine (CAS No.: 50-55-5), Retinoic acid p-hydroxyanilide (CAS No.: 65646-68-6), Ro 19-4605, S 14506 hydrochloride (CAS No.: 286369-38-8), S(−)-Atenolol (CAS No.: 93379-54-5), SANT-1 (CAS No.: 304909-07-7), SB 203580 (CAS No.: 152121-47-6), SB 206553 hydrochloride (CAS No.: 1197334-04-5), SB 228357 (CAS No.: 181629-93-6), SB 239063 (CAS No.: 193551-21-2), SB 408124 (CAS No.: 288150-92-5), SB 415286 (CAS No.: 264218-23-7), securinine (CAS No.: 5610-40-2), SKF 77434 hydrobromide (CAS No.: 300561-58-4), SKF 86002 dihydrochloride (CAS No.: 116339-68-5), Sorafenib (CAS No.: 284461-73-0), thioguanine (CAS No.: 154-42-7), Topotecan hydrochloride hydrate (CAS No.: 119413-54-6), Triamterene (CAS No.: 396-01-0), Trifluridine (CAS No.: 70-00-8), tulobuterol (CAS No.: 41570-61-0), Tyrphostin B44 (−) enantiomer (CAS No.: 133550-32-0), Vinblastine (CAS No.: 865-21-4), Vorinostat (CAS No.: 149647-78-9), VU 0155069 (CAS No.: 1130067-06-9), derivatives thereof, and combinations thereof.

The presently disclosed subject matter also provides for methods of treating ciliopathies. In certain embodiments, a method of treating a ciliopathy comprises administering to a subject suffering from, diagnosed as having, or at risk of having the ciliopathy, an effective amount of one or more cilium elongation modulator, which regulates expression of a gene in a cell by modulating a length of one or more primary cilia of the cell.

In certain embodiments, the one or more cilium elongation modulator is administered in an amount effective to increase the length of one or more primary cilia of the cell.

In certain embodiments, the one or more cilium elongation modulator is administered in an amount effective to increase expression of one or more gene by the cell, for example, an osteogenic gene, as described herein.

In certain embodiments, the cilium elongation modulator regulates gene expression by modulating mechanosensitivity of the cell.

In certain embodiments, the one or more cilium elongation modulator is administered in an amount effective to increase mechanosensitivity of the cell.

In certain embodiments, increasing the one or more cilium elongation modulator is administered in an amount effective to increase expression of an osteogenic gene, and increases osteogenesis.

In certain embodiments, the ciliopathy is Alstrom syndrome, Bardet-Biedl syndrome, Joubert syndrome, Meckel-Gruber syndrome, nephronophthisis, orofaciodigital syndrome, Senior-Loken syndrome, autosomal dominant polycystic kidney disease (ADPKD) and autosomal recessive polycystic kidney disease (ARPKD), Kartagener Syndrome, asphyxiating thoracic dysplasia, Marden-Walker syndrome, or any combination thereof.

The presently disclosed subject matter also provides for methods of treating osteoporosis. In certain embodiments, a method of treating osteoporosis comprises administering to a subject suffering from, diagnosed with, or at risk of having osteoporosis an effective amount of a cilium elongation modulator, which regulates expression of a gene in a cell by modulating a length of one or more primary cilia of the cell.

In certain embodiments, the one or more cilium elongation modulator is administered in an amount effective to increase the length of one or more primary cilia of the cell.

In certain embodiments, the one or more cilium elongation modulator is administered in an amount effective to increase expression of one or more gene by the cell, for example, an osteogenic gene, as described herein.

In certain embodiments, the cilium elongation modulator regulates gene expression by modulating mechanosensitivity of the cell.

In certain embodiments, the one or more cilium elongation modulator is administered in an amount effective to increase mechanosensitivity of the cell.

In certain embodiments, increasing the one or more cilium elongation modulator is administered in an amount effective to increase expression of an osteogenic gene, and increases osteogenesis.

The presently disclosed subject matter also provides for kits comprising one or more cilium elongation modulator, as described herein.

### BRIEF DESCRIPTION OF THE FIGURES

FIGS. 1A-1E. Small molecule treatments increase primary cilia length. 10 μM fenoldopam (A) and 500 μM lithium (B) treatment for 16 h significantly increases primary cilia length compared to vehicle control. Drug treatments elicit no change in cell viability, as assessed by MTT assay (C) and no gross morphological differences are exhibited (D,E). Mean±SEM; n>25 cilia for each group, n=4 for MTT assay; ** p<0.01, ***p<0.001; scale bars=20 μm.

FIGS. 2A-2D. Cells with longer cilia are more mechanosensitive. Cells were subjected to fluid flow for 1 hour, and the fold change of flow vs no flow control groups was compared. Cells expressed significant increases in COX-2 (A, B) and OPN (C, D) mRNA relative to GAPDH endogenous control when treated with either fenoldopam (A, C) or LiCl (B, D) for 16 hours. Mean±SEM; n≥5 for each group; *p<0.05, **p<0.01, ***p<0.001.

FIGS. 3A-3C. Treatment with IFT88 siRNA disrupts primary cilia formation. Overlays of primary cilia (green) and nuclei (blue) illustrate primary cilia incidence. (A) Scramble control siRNA treatment for 48 hours does not disrupt primary cilia formation. (B) IFT88 siRNA treatment results in decreased cilia length and incidence. (C) Fenoldopam treatment recovers primary cilia formation in IFT88 siRNA treated cells. Scale bars=10 μm.

FIGS. 4A-4E. Fenoldopam rescues ciliogenesis and mechanosensing. Cells treated with IFT88 siRNA have decreased cilia length compared to scramble control, while fenoldopam treatment appears to recover cilia length (n≥25 for scramble and fenoldopam treated, n=15 for IFT88 siRNA alone) (A). Oscillatory fluid flow was applied to cells treated with IFT88 or scramble control siRNA. Impaired cilia display a decreased OPN response to fluid flow, while fenoldopam treatment is able to recover flow stimulated OPN expression (B). Treatment with IFT88 siRNA decreases cilia incidence, but is recovered with fenoldopam treatment; n>8 fields of view (C). Fenoldopam treatment on untransfected cells has no effect on cilia incidence; n≥8 fields of view (D). Fenoldopam also did not alter IFT88 mRNA expression in untransfected cells (E). Mean±SEM; n≥4; ***p<0.001.

FIGS. 5A-5F. Fenoldopam enhances adenylyl cyclase production and activity. Cells were treated with fenoldopam or vehicle control for 16 h and then forskolin stimulated for 20 min. Fenoldopam treatment significantly increases the cAMP response to forskolin stimulation (A). Fenoldopam treatment significantly increases AC6 mRNA expression (B). AC6 knockdown decreases cilia length, but is not recovered with fenoldopam treatment (C). Neither AC6 siRNA nor fenoldopam alter cilia incidence; n>8 fields of view (D). Oscillatory flow applied to AC6 siRNA treated cells elicits a decrease in AC6 and OPN mRNA expression and is not recovered with fenoldopam treatment (E,F). Mean±SEM; n≥4 for each group; * p<0.05, *** p<0.001.

FIG. 6. Osteoblast osteogenic gene expression is enhanced by culture with conditioned media from mechanically.

FIGS. 7A-7D. (A-C) Osteocytes with longer primary cilia are more mechanosensitive. (D) Osteoblast osteogenic gene expression is diminished by disruption of osteocyte primary cilia formation.

FIG. 8. Paracrine signaling to osteoblasts is altered by pharmacologically targeting osteocyte primary cilia-mediated mechanotransduction.

FIGS. 9A-9E. (A) Fenoldopam treatment enhances load-induced bone formation. (B) Load-induced bone formation assessed by dynamic histomorphometry. Alizarin (red) was administered four days after calcein (green). (C-E) Minimal adverse effects of drug treatment. There is no difference in visible bone ultrastructure between fenoldopam and vehicle control mice (FIG. 9C). Mouse weight, kidney weight, and kidney morphology assessed by H&E stain, remained unchanged in drug vs vehicle control (FIG. 9D-E). μCT analysis also revealed no change in normal bone properties due to drug treatment.

FIG. 10. Mice treated with cilia stiffening and lengthening agents and a TRPV4 channel agonist show signs of altered load-induced bone formation. All data are shown in reference to the non-loaded contralateral limb as control. Skeletally mature wildtype C57BL/6 mice subcutaneously injected with 4αPDD (250 μg/kg), fenoldopam (2 and 20 mg/kg), tubastatin (5 mg/kg), or vehicle control for 6 consecutive days. On day 4-6 the mice were subjected to daily axial compressive ulnar loading. Mice were placed under isoflurane anesthesia and forelimbs were placed between two loading cups controlled by an electromagnetic loading system with feedback control (Bose, ELF 3220). An initial 0.1 N preload was applied, followed by 3 N of cyclic compression applied with a 2 Hz sine wave for 120 cycles, generating a strain gage verified average peak deformation of 1895 μstrain. On day 8, mice were subcutaneously injected with calcein (10 mg/kg) and alizarin red (70 mg/kg) on day 12. Mice were euthanized on day 18 and prepared for dynamic histomorphometric analysis. 4αPDD and fenoldopam treatment both displayed similar increases in bone formation rate (rBFR/BS), compared to vehicle control. Due to power limitations, tubastatin treatment did not elicit a marked change (Vehicle n=13, tubastatin n=3, 4αPDD n=3, fenoldopam (2 mg/kg) n=12, fenoldopam (20 mg/kg) n=2, Mean±SEM).

FIG. 11. Ciliary cAMP increases following a spike in ciliary calcium influx. Osteocyte-like MLO-Y4 cells transfected with a cAMP biosensor demonstrate increased cAMP levels in the cilium in response to flow. Cells were electroporated with cAMP and calcium biosensors, seeded on collagen coated slides, treated with reduced-serum media for 3 days, and exposed to 1 Hz 10 dynes/cm² oscillatory fluid flow (OFF). A Quad-view beam splitter was used to collect ECFP, YFP, and mApple fluorescence every 0.5 seconds, simultaneously measuring ciliary cAMP and calcium influx (cAMP n=6, calcium n=8,*p<0.05, Mean±SEM).

FIGS. 12A-12B. (A) Calcium inhibition of AC6 is necessary for normal flow-induced cAMP production and osteogenic response in osteocytes. Cells transfected with mutant AC6 demonstrate increased cAMP production and decreased COX-2 response. Cells were transfected with pcDNA3.2, pcDNA3.2+AC6, or pcDNA3.2+AC6^{CalMut} and received 600 ug/mL Geneticin for 2 days followed by 2 days of reduced-serum media. 5 days PE, cells were placed into flow chambers, acclimated for 30 minutes, exposed to flow for 2 minutes, and lysed to quantify cAMP. RNA was isolated 1 hour after 30 minutes of OFF and COX-2 expression was quantified via RT-qPCR. Flow measurements were normalized to static controls. (n=3, *p<0.05, Mean±SEM). (B) Calcium binding inhibition of TRPV4 and AC6 reduce osteocyte osteogenic response to flow, while it promotes osteogenic response for AC3. Cells overexpressing TRPV4 and AC6 have increased COX-2 response to flow, but this is abrogated in mutants. AC3 overexpression has a decreased response to flow, which is also prevented by the binding pocket mutation. Data is shown as COX-2 expression of the experimental plasmid flow to no-flow response over the control plasmid flow to no-flow response. Cells were transfected with pcDNA3.2, pcDNA3.2+TRPV4, pcDNA3.2+TRPV4^{CaMMut}, pcDNA3.2+AC6, pcDNA3.2+AC6^{CalMut,} pcDNA3.2+AC3, or pcDNA3.2+AC3^{CalMut} and received 600 ug/mL Geneticin for 2 days followed by 2 days of reduced-serum media. 5 days PE, cells were placed into flow chambers, acclimated for 30 minutes, exposed to flow for 5 minutes. RNA was isolated 1 hour after OFF and COX-2 expression was quantified via RT-qPCR. (n=6, *p<0.05, **p<0.01, Mean±SEM).

FIG. 13. Ciliary AC6 is depleted by disrupting a localization sequence. Cells containing a mutated intracellular V×P motif lacked AC6 localization to the cilium. Cells were transfected as in FIG. 3 and immunocytochemistry (ICC) was performed (4 days PE) using primary antibodies against the V5 tag of pCDNA3.2 to visualize construct expression and Arl13b to detect cilia (100× magnification).

FIG. 14. AC3 localizes to the osteocyte cilium but is absent from kidney cilia. MLO-Y4 and IMCD cells were seeded on collagen and fibronectin coated glass bottom dishes, respectively, at similar confluences. Cells were fixed and double ICC was performed with primary antibodies against AC3 and acetylated α-tubulin to identify cilia (100× magnification).

FIGS. 15A-15D. Repetitive calcium peaks are seen in an osteocyte network in response to mechanical loading. Tibiae were dissected and allowed to recover in MEMα, 10% FBS, 10% FCS for two hours and incubated with Fluo-8 AM. A preliminary sequence of 100 cyclic loads were applied (2 N preload with 8 N peak-peak amplitude). After the preload, specimens were allowed to recover for 15 mins. Then, 10 mins of 4 sec rest-inserted loading was applied and the calcium signal was measured with a 473 nm laser at the rests. (A) Osteocyte network 40 μm below the medial proximal surface with over 40 cells imaged simultaneously (10× objective). (B) Expanded view of inset illustrating one osteocyte with a basal calcium signal and (C) the same cell with increased signal intensity after loading, and the time-history (D) of the same osteocyte with three distinct calcium peaks.

FIG. 16. Cyclic overloading of murine ulnae increases osteocyte RANKL/OPG expression indicating osteoclastogenesis. Under isofluorane anesthesia, the right ulnae were loaded, similar to Study 1.1. An initial cyclic load of 0.5 N at 2 Hz was applied to seat ulnae into the fixture. Then, 10 cycles of loading were applied at 2 N. Displacement was scaled based on previous strain gauge studies to apply approximately 3000 μstrain. Displacement-controlled loading was carried out until reaching a 20% decrease in stiffness. Five days post-loading, unlae were dissected, snap frozen with liquid nitrogen, ground with a mortar and pestle and total RNA was isolated. Results are presented as the relative levels of RANKL over OPG (n=4, Mean±SEM).

FIGS. 17A-17B. (A) Schematic of the TRPV4 linked calcium biosensor (TRPV4-L-CaB) construction. An Xbal restriction site is introduced to a FRET-based calcium biosensor to cut and ligate TRPV4 into the plasmid. An additional mutation after L-CaB will block an Xbal site. (B) Completed TRPV4 linked calcium biosensor. A similar strategy will be employed to add Arl13b to TRPV4.

FIG. 18 is a schematic of a high-throughput screening platform. An automated platform was developed to perform a complete high-throughput drug screening and analysis. First, cells are seeded on 384-well plates, then treated with one of 6931 compounds with known biologic activity from commercially available small molecule libraries. Cells are then fixed, stained, and imaged. Images are then analyzed using custom MATLAB scripts, and compounds that increase cilia length were classified based on mechanism of action.

FIGS. 19A, 19B, 19C and 19D are images of exemplary primary cilia imaging, detection, graphs reporting and exemplary analysis thereof.

FIG. 20A is a graph reporting exemplary classification of compounds that increase cilia length.

FIG. 20B is a graph reporting exemplary classification of compounds that increase cilia incidence.

FIG. 21 is a schematic of compounds conserved between lists of hits independently increasing cilia length and incidence are classified based on mechanism of action. Below each class is also listed the number of conserved compounds in each group, 18 in total. Anti-folates (DHFR inhibitors) are the most commonly conserved, with topoisomerase, HDAC, and DNA/RNA synthesis inhibitors all having multiple conserved hits.

FIG. 22 is a schematic illustrating exemplary flow-induced osteogenic paracrine signaling.

FIG. 23 is a schematic illustrating an exemplary model of flow-induced paracrine signaling.

FIG. 24 is a graph reporting exemplary results of MSC osteogenic differentiation by pharmacologically manipulating osteogenic intercellular communication.

FIG. 25 is a schematic of the injection and loading timeline of an experiment investigating targeting primary cilia to promote load-induced bone formation in vivo. 16 week old mice were subcutaneously injected with fenoldopam (20 or 50 mg/kg) or vehicle control on 7 consecutive days. On the final 3 days mice were also subjected to compressive ulnar loading. Schematic of mouse ulna placed between loading platens was adapted from Warden and Turner, Bone 34, 261-270 (2004). 2 days following the completion of applied load, mice were injected with calcein, and then alizarin 4 days later. These fluorochrome labels allow quantification of regions of newly deposited bone mineral for analysis of load-induced bone formation rates.

FIG. 26 is a set of exemplary fluorescence microscopy images showing fluorochrome labeling of new bone formation.

FIGS. 27A-27C are a set of graphs reporting exemplary data on mineralizing surface (Panel A), mineral apposition rate (Panel B) and bone formation rate (Panel C) in mice treated with vehicle, fenoldopam, or high-dose fenoldopam (Fen-high).

FIG. 28 is a graph reporting exemplary data of AC6 mRNA expression in ulnae.

FIG. 29 is a set of images showing exemplary results of analysis of bone ultrastructure and microarchitecture.

FIGS. 30A-30B are a set of graphs reporting exemplary data on kidney weight (Panel A) and liver weight (Panel B) in mice treated with high dose fenoldopam (Fen-High) or vehicle.

FIG. 31 is a set of images showing exemplary histology results of kidney and liver from mice treated with fenoldopam or vehicle.

FIG. 32 is a graph reporting exemplary data on urine creatinine in mice treated with fenoldopam or vehicle.

FIGS. 33A-33C are a set of graphs reporting exemplary data on mineralizing surface (Panel A), mineral apposition rate (Panel B) and bone formation rate (Panel C) in osteoporotic mice treated with vehicle or fenoldopam.

### DETAILED DESCRIPTION

The presently disclosed subject matter relates to methods of regulating gene expression in a cell by modulating the length of primary cilia of the cell. The present subject matter also provides for methods of treating ciliopathies and osteoporosis. For purposes of clarity of disclosure and not by way of limitation, the detailed description is divided into the following subsections:

1. Definitions;

2. Method of Regulating Gene Expression by Modulating Cilium Length;

3. Method of Treating Ciliopathies;

4. Method of Treating Osteoporosis; and

5. Kits.

### 1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “derivative” refers to chemical compounds with a similar or the same core structure.

As used herein, the term “cell culture” refers to a growth of cells in vitro in an artificial medium for research or medical treatment.

As used herein, the term “expressing” in relation to a gene or protein refers to generating an mRNA and/or an amino acid sequence from a nucleic acid template, for example, a gene, which can be observed using assays such as microarray assays, antibody staining assays, and the like.

As used herein, the term “ciliopathy” refers to genetic disorders caused by dysfunctional cellular cilia. Ciliopathies include, but are not limited to, Alstrom syndrome, Bardet-Biedl syndrome, Joubert syndrome, Meckel-Gruber syndrome, nephronophthisis, orofaciodigital syndrome, Senior-Loken syndrome, polycystic kidney disease (e.g. autosomal dominant polycystic kidney disease (ADPKD) and autosomal recessive polycystic kidney disease (ARPKD), Kartagener Syndrome, asphyxiating thoracic dysplasia, Marden-Walker syndrome, and situs inversus.

As used herein, the term “osteoporosis” refers to a disease causing decreased bone strength and higher risk of a broken bone, compared to a subject that does not have osteoporosis.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the terms “regulates,” “modulates” or “modifies” refers to an increase or decrease in the amount, quality or effect, for example, of a particular expression of a gene, length of a cilium, or mechanosensitivity.

An “effective amount” of a substance as that term is used herein is that amount sufficient to effect beneficial or desired results, including clinical results, and, as such, an “effective amount” depends upon the context in which it is being applied. In the context of administering a composition to modulate expression of a gene, an effective amount of a composition is an amount sufficient to increase or decrease the expression of the gene. For example, the decrease can be a 1%, 5%, 10%, 20%, 30%, 40%, 50%, 60%, 70%, 80%, 90%, 95%, 98%, 99% or 100% decrease in the gene expression; the increase can be a 1%, 5%, 10%, 20%, 30%, 40%, 50%, 60%, 70%, 80%, 90%, 100%, 150%, 200%, 300%, 400%, 500%, 1000% or more increase in the gene expression. An effective amount can be administered in one or more administrations.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

### 2. Method of Regulating Gene Expression by Modulating Cilium Length

The presently disclosed subject matter relates to methods of regulating gene expression in a cell by modulating the length of primary cilia of the cell.

In certain embodiments, the methods of regulating expression of a gene in a cell comprise administering to the cell an effective amount of one or more cilium elongation modulator, wherein the cilium elongation modulator modulates a length of one or more primary cilia of the cell.

In certain embodiments, the one or more cilium elongation modulator is contacted to the cell in an amount effective to increase the length of one or more primary cilia of the cell.

In certain embodiments, the one or more cilium elongation modulator is contacted to the cell in an amount effective to increase expression of one or more genes of the cell.

Primary cilium is a non-motile sensory cellular organelle with a 9+0 microtubule formation. Primary cilium is found on the surface of almost all mammalian cell types.

Primary cilium is assembled based on centrosome or basal bodies in quiescent cells, and it is disassembled when cells re-enter the cell cycle. Genes that control cilium assembly and cell cycle include AKT1, BBS4, CCND1, CDK5RAP2, CDKN1A (P21CIP1/WAF1), IGF1, INS2, MAP2K1, PKD1, PKD2 and TRP53. Molecule transportation within intraflagellar space and between intraflagellar space and the rest of cell plasma is important for cilium assembly and elongation. Genes essential for such transportation include, for example, DYNC2LI1, IFT172, IFT20, IFT74, IFT80, IFT88 and Kinesin-like protein (KIF3A, KIF3B). Other genes involved in cilium formation include ALMS1, ARL6, BBS1, BBS2, BBS4, BBS7, IFT172, IFT88, MKKS, OFD1, PKHD1, RPGRIP1L, VANGL2 and WWTR1. In certain embodiments, the cilium elongation modulator is an agent which modulates, for example, increases, the gene expression and/or protein level of one or more of the genes involved in cilium assembly, cell cycle, and/or intraflagellar transportation.

In certain embodiments, the cilium elongation modulator modulates the length of primary cilia of by modulating the gene expression level and/or protein level of one or more of the genes involved in cilium assembly, cell cycle, and/or intraflagellar transportation. In certain embodiments, the cilium elongation modulator increases the length of primary cilia of by increasing the gene expression level and/or protein level of one or more of the genes involved in cilium assembly, cell cycle, and/or intraflagellar transportation. In certain embodiments, the one or more gene involved in cilium assembly, cell cycle, and/or intraflagellar transportation is selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

Primary cilia maintain lengths from 1 to 10 μm in mammalian cells. Intracellular signaling pathways can affect cilia length control. One cilium length control effector is cAMP and its associated calcium signaling pathway. Increased cyclic AMP levels and PKA activity in cells can stimulate the growth of cilia. Adenylate cyclase (AC) knockdown or reduction can block or inhibit cilium length increase.

Lithium, an inhibitor of glycogen synthase kinase 3β (GSK3β) can cause cilium elongation by decreasing GSK3β activity. The NIMA related kinase (Nek) family kinases are also involved in regulating cilia length control and congenital mutations on them cause ciliopathies. Nek family kinases have 11 members and Nek1 and 8 can also influence cilium growth.

Signal transduction pathways are also involved in cilia length control at different steps of ciliogenesis, including, but not limited to, FGF signaling. The Mammalian target-of-rapamycin (mTOR) pathways can also modulate ciliary length. Suppression of mTOR pathway by rapamycin can result in short cilia and pathway activation increases the length of cilia. In addition, MAK, CCRK CDC14 also affects ciliogenesis.

Cell shape and contractility genes can also determine cilium length. For example, polymerization of actin filaments suppresses cilia formation. Gelsolin family members, e.g., GSN and AVIL, can promote cilia formation. ACTR3, which polymerizes actin filaments, can inhibit ciliation, while its depletion can increase cilium length. Likewise, cytochalasin D, a drug that inhibits actin filament polymerization, can increase the length of the cilia. Jasplakinolide, a drug that induces the formation of actin filaments, can also lead to an increase in cilium length. Additionally the orientation of the actin cytoskeleton and the level of stress fiber formation can have a significant impact on cilium length.

In certain embodiments, the one or more cilium elongation modulator is administrated in a effective amount to modulate the gene expression of one or more signal pathway genes associated with primary cilia. Primary cilia are a nexus of cell signaling, associated with regulation of the Notch, Hedgehog, PDGF, TOR and Wnt signaling pathways. Components of these signaling pathways are concentrated within the ciliary compartment, which promotes efficient signal transduction. Signal pathway genes associated with primary cilia include
Hedgehog pathway: BTRC (BTRCP), FUZ, GLI1, GLI2, GLI3, GSK3B, IHH, INTU, LRP2, PTCH1, RAB23, SHH, SMO and SUFU;
cAMP pathway: ADCY3, ADCY7, AVPR2, HTR6, PKD2 and SSTR3;
mTOR pathway: AKT1, CDC42, GSK3B, IGF1, INS2, MAPK1, MTOR, PIK3CA (P110A), PRKCA, RHOA, TRP53, TSC1 and TSC2;
Planar Cell Polarity pathway: DVL1, FAT4, FJX1, FUZ, FZD1, INTU, RHOA, ROCK2, VANGL2 and WNT9B;
WNT pathway: AXIN2, GSK3β and INVS; and
MAP Kinase pathways: FOS, KRAS, MAP2K1 (MEK1), MAPK1 (ERK2), MOS, PRKCA and PTPN5.

In certain embodiments, the one or more cilium elongation modulator regulates gene expression by modulating mechanosensitivity of the cell. In certain embodiments, the deflection of the primary cilium can cause an increase in intracellular calcium. Such calcium response can be mediated by a mechanosensory complex located at the base of the cilium, comprising Polycystin 1 and Polycystin 2. For example, the primary cilium can function as a mechanosensor in bone cells, for example, osteocytes, osteoblasts, osteoclasts, osteoprogenitor cells, or a combinations thereof, where deflection of the primary cilium, for example, under fluid flow can increase expression of osteogenic genes as described herein, for example, COX-2 and/or OPN. In certain embodiments, deflecting a primary cilium induces a rapid and transient decrease in cAMP.

In certain embodiments, the cilium elongation modulator modulates the length of one or more primary cilia of the cell by modulating, for example, decreasing, cAMP level or activity.

Cyclic adenosine monophosphate (cAMP or cyclic AMP) is a derivative of adenosine triphosphate (ATP) and is involved in intracellular signal transduction. Cyclic AMP is synthesized by adenylate cyclase, a 12-transmembrane glycoprotein that catalyzes ATP to form cAMP. The cAMP produced is a second messenger in cellular metabolism and is an allosteric activator of protein kinase A (PKA). Adenylate cyclase is activated by signaling molecules through the activation of adenylate cyclase stimulatory G-protein-coupled receptors. Adenylate cyclase is inhibited by agonists of adenylate cyclase inhibitory G protein-coupled receptors. cAMP signals can be terminated by cAMP phosphodiesterase, an enzyme that degrades cAMP and inactivates protein kinase A.

In certain embodiments, the cilium elongation modulator modulates, for example, decreases, an expression level or an enzymatic activity of adenylyl cyclase, by which the cAMP level or activity in the cell is modulated.

In certain embodiments, the cilium elongation modulator comprises fenoldopam, lithium, derivatives thereof, or combinations thereof.

In certain embodiments, the cilium elongation modulator comprises an adenylyl cyclase agonist. In certain embodiments, the cilium elongation modulator is selected from the group consisting of fenoldopam, forskolin, NKH 477 (CAS No: 138605-00-2), PACAP 1-27 (CAS No: 127317-03-7), PACAP 1-38 (137061-48-4), and combinations thereof.

In certain embodiments, the cilium elongation modulator comprises a dopamine D1-like receptor agonist. In certain embodiments, the cilium elongation modulator is selected from the group consisting of fenoldopam, Dihydrexidine (CAS No: 158704-02-0), Dopamine (CAS No: 62-31-7), NPEC-caged-dopamine (CAS No: 1257326-23-0), SKF 38393 (CAS No: 20012-10-6), SKF 77434 (CAS No: 300561-58-4), SKF 81297 (CAS No: 67287-39-2), SKF 82958 (CAS No: 74115-01-8), SKF 83822 (CAS No: 74115-10-9), SCH-23390 (CAS No: 87075-17-0), SKF-83959 (CAS No: 67287-95-0), A68930 (CAS No: 130465-39-3), A77636 (CAS No: 145307-34-2), (R)-(−)-Apomorphine (CAS No: 314-19-2), CY 208-243 (CAS No: 100999-26-6), Ecopipam (SCH-39,166, CAS No: 112108-01-7), and combinations thereof.

In certain embodiments, the cilium elongation modulator is lithium.

Example 4 describes results of a high-throughput drug screen that was utilized to recognize compounds that manipulate ciliogenesis. Example 4 describes exemplary small molecules, as well as classes of compounds based on mechanism of action, that may be used as cilium elongation modulators.

In certain embodiments, the cilium elongation modulator may be selected from a dihydrofolate reductase antagonist, an inhibitor of DNA synthesis or RNA synthesis, a glycogen synthase kinase 3 inhibitor, a topoisomerase inhibitor, a nucleoside analogue, an HDAC inhibitor, a 5-HT modulator, an anthelmintic, an epidermal growth factor receptor kinase inhibitor, a histamine receptor modulator, a dopamine D1 receptor agonist, a checkpoint kinase 1 inhibitor, a microtubule stabilizer, a reactive oxygen species modulator, a sodium channel blocker, an adrenoreceptor modulator, an inosine-5′-monophosphate dehydrogenase inhibitor, a benzodiazepine inverse agonist, an opioid receptor modulator, and an antiseptic.

In certain embodiments, the cilium elongation modulator may be selected from (S)-(+)-Niguldipine hydrochloride (CAS No.: 113145-69-0), 10-hydroxycamptothecin (CAS No.: 19685-09-7), 110-Phenanthroline monohydrate (CAS No.: 5144-89-8), 2 4-dihydroxychalcone 4-glucoside (CAS No.: 25515-43-9), 2-Methoxyestradiol (CAS No.: 362-07-2), 5-BDBD (CAS No.: 768404-03-1), 5-fluorouracil (CAS No.: 51-21-8), 62-dimethoxyflavone, 6-aminonicotinamide (CAS No.: 329-89-5), AG 494 (CAS No.: 139087-53-9), AM 630 (CAS No.: 164178-33-0), Amethopterin (R,S) (CAS No.: 60388-53-6), aminopterin (CAS No.: 54-62-6), BAY 61-3606 hydrochloride hydrate (CAS No.: 732938-37-8), Benzethonium chloride (CAS No.: 121-54-0), betahistine hydrochloride (CAS No.: 15430-48-5), beta-toxicarol (CAS No.: 82-11-1), BIO (CAS No.: 667463-62-9), Bromhexine HCl (CAS No.: 3572-43-8), Bufexamac (CAS No.: 2438-72-4), BW 373U86 (CAS No.: 155836-50-3), CP 339818 hydrochloride (CAS No.: 478341-55-8), CPT 11 (CAS No.: 100286-90-6), CY 208-243 (CAS No.: 100999-26-6), cycloheximide (CAS No.: 66-81-9), Cyclosporin A (CAS No.: 59865-13-3), deacetoxy-7-oxogedunin (CAS No.: CAS No. 13072-74-7), DEGUELIN(−) (CAS No.: 522-17-8), dehydrocholic acid (CAS No.: 81-23-2), DEOXYGEDUNIN (CAS No.: 21963-95-1), deoxykhivorin, deoxysappanone b 73-dimethyl ether acetate (CAS No.: 113122-54-6), dihydrocelastryl diacetate, Diphenyleneiodonium chloride (CAS No.: 4673-26-1), Docetaxel (CAS No.: 114977-28-5), Dorzolamide HCl (CAS No.: 130693-82-2), Droxinostat (CAS No.: 99873-43-5), duartin dimethyl ether, estradiol cypionate (CAS No.: 313-06-4), Ethacridine lactate monohydrate (CAS No.: 1837-57-6), Etonitazenyl isothiocyanate (CAS No.: 85951-65-1), Floxuridine (CAS No.: 50-91-9), Ginkgolide B (CAS No.: 15291-77-7), GW 1929 (CAS No.: 196808-24-9), harmalol hydrochloride (CAS No.: 6028-07-5), hecogenin acetate (CAS No.: 915-35-5), homidium bromide (CAS No.: 1239-45-8), hycanthone (CAS No.: 3105-97-3), IC 261 (CAS No.: 186611-52-9), Indirubin-3′-oxime (CAS No.: 160807-49-8), Irinotecan (CAS No.: 97682-44-5), itraconazole (CAS No.: 84625-61-6), Kenpaullone (CAS No.: 142273-20-9), khivorin (CAS No.: 2524-38-1), levulinic acid 3-benzylidenyl-, Lonidamine (CAS No.: 50264-69-2), Loratidine (CAS No.: 79794-75-5), Merbromin (CAS No.: 129-16-8), mercaptopurine (CAS No.: 50-44-2), Methotrexate (CAS No.: 59-05-2), methoxyamine hydrochloride (CAS No.: 67-62-9), monensin sodium (CAS No.: 22373-78-0), monobenzone (CAS No.: 103-16-2), Mycophenolate mofetil (CAS No.: 128794-94-5), Oxfendazole (CAS No.: 53716-50-0), pararosaniline pamoate (CAS No.: 7460-07-3), PD 169316 (CAS No.: 152121-53-4)), PD-407824 (CAS No.: 622864-54-4), Pemetrexed (CAS No.: 137281-23-3), perillic acid (−) (CAS No.: 23635-14-5), PHA 767491 hydrochloride (CAS No.: 942425-68-5), PIM 1 Inhibitor 2 (CAS No.: 477845-12-8), Piperlongumine (CAS No.: 20069-09-4), Pralatrexate (CAS No.: 146464-95-1), pyrimethamine (CAS No.: 58-14-0), pyrvinium pamoate (CAS No.: 3546-41-6), quinidine gluconate (CAS No.: 56-54-2), quinine sulfate (CAS No.: 6119-70-6), reserpine (CAS No.: 50-55-5), Retinoic acid p-hydroxyanilide (CAS No.: 65646-68-6), Ro 19-4605, S 14506 hydrochloride (CAS No.: 286369-38-8), S(−)-Atenolol (CAS No.: 93379-54-5.), SANT-1 (CAS No.: 304909-07-7), SB 203580 (CAS No.: 152121-47-6), SB 206553 hydrochloride (CAS No.: 1197334-04-5), SB 228357 (CAS No.: 181629-93-6), SB 239063 (CAS No.: 193551-21-2), SB 408124 (CAS No.: 288150-92-5), SB 415286 (CAS No.: 264218-23-7), securinine (CAS No.: 5610-40-2), SKF 77434 hydrobromide (CAS No.: 300561-58-4), SKF 86002 dihydrochloride (CAS No.: 116339-68-5), Sorafenib (CAS No.: 284461-73-0), thioguanine (CAS No.: 154-42-7), Topotecan hydrochloride hydrate (CAS No.: 119413-54-6), Triamterene (CAS No.: 396-01-0), Trifluridine (CAS No.: 70-00-8), tulobuterol (CAS No.: 41570-61-0), Tyrphostin B44 (−) enantiomer (CAS No.: 133550-32-0), Vinblastine (CAS No.: 865-21-4), Vorinostat (CAS No.: 149647-78-9), VU 0155069 (CAS No.: 1130067-06-9), derivatives thereof, and combinations thereof.

In certain embodiments, the cilium elongation modulator modulates the length of primary cilia of by modulating the gene expression level and/or protein level of one or more of the genes involved in cilium assembly, cell cycle, and/or intraflagellar transportation. In certain embodiments, the cilium elongation modulator increases the length of primary cilia by increasing the gene expression level and/or protein level of one or more of the genes involved in cilium assembly, cell cycle, and/or intraflagellar transportation. In certain embodiments, the one or more gene involved in cilium assembly, cell cycle, and/or intraflagellar transportation is selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

In certain embodiments, the cilium elongation modulator comprises a nucleic acid sequence encoding one or more proteins, or functional fragments thereof, involved in cilium assembly, cell cycle, and/or intraflagellar transportation, signal transduction, cell shape and contractility, as described herein.

In certain embodiments, the cilium elongation modulator comprises an amino acid sequence of a protein, or functional fragment thereof, involved in cilium assembly, cell cycle, and/or intraflagellar transportation, signal transduction, cell shape and contractility, as described herein.

In certain embodiments, the cilium elongation modulator decreases the length of primary cilia of by decreasing the gene expression level and/or protein level of one or more of the genes involved in cilium assembly, cell cycle, and/or intraflagellar transportation. In certain embodiments, the cilium elongation modulator comprises a compound that can decrease expression or activity of one or more genes or proteins involved in cilium assembly, cell cycle, and/or intraflagellar transportation, signal transduction, cell shape and contractility, as described herein. Such compounds can include, for example, an RNAi molecule, antibody or fragment thereof capable of targeting one or more gene involved in cilium assembly, cell cycle, and/or intraflagellar transportation, signal transduction, cell shape and contractility. Examples of RNAi molecules include, but are not limited to, the following: siRNA, shRNA, microRNA, double stranded RNA, as well as any modifications or derivatives thereof.

Modulation of gene expression can be accomplished by a recombinant DNA construct. In certain embodiments, a vector (e.g., a non-viral vector or a rival vector, e.g., a gamma-retroviral or lentiviral vector) can be employed for the introduction of the DNA construct into the cell. For example, a polynucleotide encoding a genes involved in cilium assembly, cell cycle, and/or intraflagellar transportation can be cloned into a vector and expression can be driven from a endogenous promoter of the vector, or from a promoter specific for a target cell type of interest.

In certain embodiments, other viral vectors are used to modulate the expression of genes involved in cilium assembly, cell cycle, and/or intraflagellar transportation, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995).

In certain embodiments, non-viral approaches are used to modulate the expression of genes involved in cilium assembly, cell cycle, and/or intraflagellar transportation. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990).

In certain embodiments, other non-viral means are used to modulate the expression of genes involved in cilium assembly, cell cycle, and/or intraflagellar transportation. Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1c enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In addition to full-length polypeptides, the present disclosure also provides fragments of any one of the polypeptides or peptide domains of the genes involved in cilium assembly, cell cycle, and/or intraflagellar transportation. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80, 100, 200, 300 or more contiguous amino acids. Fragments of the genes can be generated by methods known to those skilled in the art or may result from normal protein processing (e.g., removal of amino acids from the nascent polypeptide that are not required for biological activity or removal of amino acids by alternative mRNA splicing or alternative protein processing events).

In certain embodiments, the cell being contacted with a cilium elongation modulator is an osteocyte, an osteoblast, an osteoclast, an osteoprogenitor cell, or combinations thereof.

In certain embodiments, the cilium elongation modulator is contacted to a population of cells.

Osteocytes are bone cells located in mature bone. Osteocytes are derived from osteoprogenitor cells. Osteocytes generate bone matrix through mechanosensory mechanisms. Osteocytes decompose bone through a rapid, transient mechanism, i.e., osteocytic osteolysis, and deposit hydroxyapatite, calcium carbonate and calcium phosphate. Osteocytes also synthesize sclerostin, a secreted protein product of the SOST gene that inhibits bone formation by binding to LRP5/LRP6 receptors and blocking Wnt signaling. Sclerostin is inhibited by parathyroid hormone (PTH) and mechanical loading. Sclerostin inhibits the activity of BMP (bone morphogenetic protein).

Osteoblasts are another type of bone cells. Osteoblasts synthesize dense, crosslinked collagen, and also synthesize osteocalcin and osteopontin, which comprise the matrix of bone.

Osteoclasts are a type of bone cells responsible for the breakdown and remodeling of bones. An osteoprogenitor cell is the precursor of the differentiated bone cells described above. Unlike the other types of bone cells, osteoprogenitor cells maintain the ability to divide.

In certain embodiments, the expression of the one or more gene modified according to the methods described herein is an osteogenic gene. Osteogenic genes include, but are not limited to, genes that promote osteogenesis. Non-limiting examples of osteogenic genes include the following:
Runt-related transcription factor 2 (Runx2, (Cbfa1/PEBP2αA/AML-3/Osf2)), Osterix (Osx), distal-less homeobox protein 5 (Dlx5), Alkaline phosphatase (ALP), Msx-2 (Hox-8), Nuclear factor of kappa light polypeptide gene enhancer in B-cells (NF-κB), Osteoprotegerin (OPG), Cytochrome c oxidase subunit 2 (Cox-2), fibroblast growth factor 2 (FGF2), bagpipe homeobox homolog 1 (Drosophila) (Bapx1), Collagen I, Osteocalcin, Osteopontin (OPN), and Bone sialoprotein (BSP).
Bone mineralization genes: AHSG, AMBN, AMELY, BGLAP, ENAM, MINPP1, STATH, and TUFT1.
Cartilage condensation genes: BMP1, COL11A1, and SOX9.
Ossification genes: ALPL, AMBN, AMELY, BGLAP, CALCR, CDH11, DMP1, DSPP, ENAM, MINPP1, PHEX, RUNX2, STATH, TFIP11, and TUFT1.
Calcium ion binding and homeostasis genes: ANXA5, BGLAP, BMP1, CALCR, CDH11, COMP, DMP1, EGF, MMP2, MMP8, and VDR.
Growth factors and receptors: BMP1, BMP2, BMP3, BMP4, BMP5, BMP6, CSF2, CSF3, EGF, EGFR, FGF1, FGF2, FGF3, FGFR1, FGFR2, FLT1, GDF10, IGF1, IGF1R, IGF2, PDGFA, TGFB1, TGFB2, TGFB3, TGFBR1, TGFBR2, VEGFA, and VEGFB.
Extracellular matrix (ECM) Molecules: COL4A3, COL10A1, COL11A1, COL12A1, COL14A1, COL15A1, COL1A1, COL1A2, COL2A1, COL3A1, COL4A3, COL5A1.
ECM protease inhibitors: AHSG, COL4A3, and SERPINH1
ECM proteases: CTSK, MMP10, MMP2, MMP8, MMP9, and PHEX.
Cell Adhesion Molecules: CDH11, COL11A1, COL14A1, ICAM1, ITGB1, VCAM1, ITGA1, ITGA2, ITGA3, ITGAM, ITGB1, BGLAP, CD36, COL12A1, COL15A1, COL4A3, COL5A1, COMP, FN1, SCARB1, and TNF.
Transcription factors: MSX1, NFKB1, RUNX2, SMAD1, SMAD2, SMAD3, SMAD4, SOX9, TNF, TWIST1, and VDR.

In certain embodiments, the cilium elongation modulator increases the length of one or more primary cilia of the cell. In certain embodiments, the cilium elongation modulator increases an expression level of one or more osteogenic gene by increasing the length of one or more primary cilia of the cell. In certain embodiments, the one or more osteogenic gene is selected from the group consisting of COX-2, OPN, BSP, Collagen I, Osteocalcin, Runt-related transcription factor 2 (Runx2, (Cbfa1/PEBP2αA/AML-3/Osf2)), Osterix (Osx), distal-less homeobox protein 5 (Dlx5), Alkaline phosphatase (ALP), Msx-2 (Hox-8), Nuclear factor of kappa light polypeptide gene enhancer in B-cells (NF-κB), Osteoprotegerin (OPG), Cytochrome c oxidase subunit 2 (Cox-2), fibroblast growth factor 2 (FGF2), bagpipe homeobox homolog 1 (Drosophila) (Bapx1), Collagen I, Osteocalcin, Osteopontin (OPN), Bone sialoprotein (BSP), AHSG, AMBN, AMELY, BGLAP, ENAM, MINPP1, STATH, TUFT1, Cartilage condensation genes: BMP1, COL11A1, SOX9, ALPL, AMBN, AMELY, BGLAP, CALCR, CDH11, DMP1, DSPP, ENAM, MINPP1, PHEX, RUNX2, STATH, TFIP11, TUFT1, ANXA5, BGLAP, BMP1, CALCR, CDH11, COMP, DMP1, EGF, MMP2, MMP8, VDR, BMP1, BMP2, BMP3, BMP4, BMP5, BMP6, CSF2, CSF3, EGF, EGFR, FGF1, FGF2, FGF3, FGFR1, FGFR2, FLT1, GDF10, IGF1, IGF1R, IGF2, PDGFA, TGFB1, TGFB2, TGFB3, TGFBR1, TGFBR2, VEGFA, VEGFB, COL4A3, COL10A1, COL11A1, COL12A1, COL14A1, COL15A1, COL1A1, COL1A2, COL2A1, COL3A1, COL4A3, COL5A1, AHSG, COL4A3, SERPINH1, CTSK, MMP10, MMP2, MMP8, MMP9, and PHEX, CDH11, COL11A1, COL14A1, ICAM1, ITGB1, VCAM1, ITGA1, ITGA2, ITGA3, ITGAM, ITGB1, BGLAP, CD36, COL12A1, COL15A1, COL4A3, COL5A1, COMP, FN1, SCARB1, TNF, MSX1, NFKB1, RUNX2, SMAD1, SMAD2, SMAD3, SMAD4, SOX9, TNF, TWIST1, VDR, and combinations thereof.

In certain embodiments, the osteogenic gene is selected from the group consisting of COX-2, OPN, BSP, Collagen I, Osteocalcin, and combinations thereof. In certain embodiments, increasing expression of one or more osteogenic genes increases osteogenesis. In certain embodiments, the cell is a mammalian cell. In certain embodiments, the mammalian cell is a human cell.

### 3. Method of Treating Ciliopathies

The presently disclosed subject matter is also directed to methods of treating ciliopathies. Human congenital disruption of cilium structure or function can cause developmental disorders. Many diseases are attributed to cilia formation and/or functional defects, which affect organs, such as kidney, brain, limb, eye, ear, liver and bone. Identified ciliopathies include, but are not limited to, Joubert syndrome (JBTS), nephronophthisis (NPHP), autosomal dominant and recessive polycystic kidney disease (ADPKD and ARPKD), Meckel-Gruber syndrome (MKS), Bardet-Biedl syndrome (BBS), Alstrom syndrome, orofaciodigital syndrome, Senior-Loken syndrome, Kartagener Syndrome, asphyxiating thoracic dysplasia, and Marden-Walker syndrome among others.

In certain embodiments, a method of treating a ciliopathy comprises administering to a subject suffering from, diagnosed with, or at risk of having a ciliopathy, an effective amount of a cilium elongation modulator, as described herein, that regulates expression of a gene in a cell of the subject by modulating a length of one or more primary cilia of the cell.

In certain embodiments, the cilium elongation modulator is administered in an amount effective to increase length of one or more primary cilia of the cell.

In certain embodiments, the cilium elongation modulator is administered in an amount effective to increase expression of a gene in the cell, wherein the gene, for example, comprises one or more osteogenic genes.

In certain embodiments, the cilium elongation modulator is administered in an amount effective to increase ostrogenesis.

In certain embodiments, the ciliopathy is Alstrom syndrome, Bardet-Biedl syndrome, Joubert syndrome, Meckel-Gruber syndrome, nephronophthisis, orofaciodigital syndrome, Senior-Loken syndrome, autosomal dominant polycystic kidney disease (ADPKD) and autosomal recessive polycystic kidney disease (ARPKD)), Kartagener Syndrome, asphyxiating thoracic dysplasia, Marden-Walker syndrome, or any combination thereof.

In certain embodiments, the cilium elongation modulator comprises one or more agents which modulate gene expression and/or protein level of one or more of the genes involved in cilium assembly, cell cycle, and/or intraflagellar transportation, as described herein. In certain embodiments, the cilium elongation modulator increases the length of primary cilia of by increasing the gene expression level and/or protein level of one or more of the genes involved in cilium assembly, cell cycle, and/or intraflagellar transportation. In certain embodiments, the one or more gene involved in cilium assembly, cell cycle, and/or intraflagellar transportation is selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

The presently disclosed cilium elongation modulator can be administered in any physiologically acceptable vehicle. Pharmaceutical compositions comprising the presently disclosed cilium elongation modulator and a pharmaceutically acceptable carrier are also provided. The presently disclosed cilium elongation modulator and the pharmaceutical compositions comprising thereof can be administered via localized injection, orthotopic (OT) injection, systemic injection, intravenous injection, or parenteral administration. In certain embodiments, the presently disclosed cilium elongation modulator are administered to a subject suffering from a ciliophacy via systemic or localized injection.

The presently disclosed cilium elongation modulator and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed cilium elongation modulator, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed cilium elongation modulators.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed cilium elongation modulator. This will present no problem to those skilled in chemical and pharmaceutical principles, or problems can be readily avoided by reference to standard texts or by simple experiments (not involving undue experimentation), from this disclosure and the documents cited herein.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the ciliopathy, or otherwise reduce the pathological consequences of the ciliopathy. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently cilium elongation modulator is an amount that is sufficient to reduce or eliminate the symptoms of a subject suffering from a ciliopathy. In certain embodiments, an effective amount of the presently disclosed cilium elongation modulator is an amount that is sufficient to prevent a subject from developing a ciliopathy.

### 4. Method of Treating Osteoporosis

The presently disclosed subject matter also provides for methods of treating osteoporosis. In certain embodiments, a method of treating osteoporosis comprises administering to a subject suffering from, diagnosed with, or at risk of having osteoporosis, an effective amount of a cilium elongation modulator, as described herein, that regulates expression of a gene in a cell by modulating a length of one or more primary cilia of the cell.

In certain embodiments, the cilium elongation modulator is administered in an amount effective to increase length of one or more primary cilia of the cell.

In certain embodiments, the cilium elongation modulator is administered in an amount effective to increase expression of a gene in the cell, wherein the gene, for example, comprises one or more osteogenic genes.

In certain embodiments, the cilium elongation modulator is administered in an amount effective to increase ostrogenesis.

Osteoporosis is a disease causing decreased bone strength and higher risk of a broken bone. Commonly affected bones include the back bones, the bones of the forearm, and the hip. There are typically no symptoms until a broken bone occurs. The WNT/Lrp pathway is a regulator of bone anabolism. In certain embodiments, the cilium elongation modulator is administered in an amount effective to increase bone strength and/or reduce risk of a broken bone, compared to a subject with osteoporosis that is not administered the cilium elongation modulator.

In certain embodiments, the cilium elongation modulator comprises one or more agents that modulates the gene expression and/or protein level of one or more of the genes involved in cilium assembly, cell cycle, and/or intraflagellar transportation, as described herein. In certain embodiments, the cilium elongation modulator increases the length of primary cilia of by increasing the gene expression level and/or protein level of one or more of the genes involved in cilium assembly, cell cycle, and/or intraflagellar transportation. In certain embodiments, the one or more gene involved in cilium assembly, cell cycle, and/or intraflagellar transportation is selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

The presently disclosed cilium elongation modulator can be administered in any physiologically acceptable vehicle. Pharmaceutical compositions comprising the presently disclosed cilium elongation modulator and a pharmaceutically acceptable carrier are also provided. The presently disclosed cilium elongation modulator and the pharmaceutical compositions comprising thereof can be administered via localized injection, orthotopic (OT) injection, systemic injection, intravenous injection, or parenteral administration. In certain embodiments, the presently disclosed cilium elongation modulator are administered to a subject suffering from a ciliophacy via systemic or localized injection.

The presently disclosed cilium elongation modulator and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed cilium elongation modulator.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed cilium elongation modulator. This will present no problem to those skilled in chemical and pharmaceutical principles, or problems can be readily avoided by reference to standard texts or by simple experiments (not involving undue experimentation), from this disclosure and the documents cited herein.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of osteoporosis, or otherwise reduce the pathological consequences of osteoporosis. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently cilium elongation modulator is an amount that is sufficient to reduce or eliminate the symptoms of a subject suffering from Osteoporosis. In certain embodiments, an effective amount of the presently disclosed cilium elongation modulator is an amount that is sufficient to prevent a subject from developing Osteoporosis.

### 5. Kits

The presently disclosed subject matter also provides for kits for regulating gene expression in a cell by modulating the length of the primary cilia of the cell. In certain embodiments, kits are provided for increasing the length of the cell's primary cilia, and increasing expression of one or more genes of the cell, for example, one or more osteogenic genes, as described herein. In certain embodiments, kits are provided for increasing the production of bone by a cell, for example, an osteoblast. In certain embodiments, the kits comprise one or more cilium elongation modulator, as described herein. In certain embodiments, the kits also comprise instructions for modulating the length of primary cilia of a cell, and thereby regulating expression of a gene in a cell, for example, increasing the length of the cell's primary cilia and increasing the expression of one or more osteogenic gene.

### EXAMPLES

The presently disclosed subject matter will be better understood by reference to the following Example, which is provided as exemplary of the presently disclosed subject matter, and not by way of limitation.

### Example 1: Lengthening Primary Cilia Enhances Cellular Mechanosensitivity

Osteocytes are mechanosensitive cells within bone, where the primary cilium functions as a mechanosensor in this context (Malone et al., 2007). In vitro, fluid flow mechanical stimulation of osteocytes enhances expression of the osteogenic genes cyclooxygenase-2, COX-2, and osteopontin, OPN. COX-2 synthesizes prostaglandin E2, and OPN is a critical extracellular matrix protein. Increases in the production of both are indicative of osteogenesis (Ehrlich and Lanyon, 2002; Fujihara et al., 2006; Klein-Nulend et al., 1997; Raisz, 1999). When osteocyte primary cilia formation is inhibited, the cells display an abrogated osteogenic response to flow, implicating the cilium as a critical mechanosensor in osteocytes (Malone et al., 2007). Furthermore, it has been previously reported that adenylyl cyclases, specifically AC6, play a significant role in osteocyte mechanosensitivity (Kwon et al., 2010). Adenylyl cyclases convert ATP to the ubiquitous second messenger cAMP, a process which can be specifically stimulated by forskolin.

Due to the significance of primary cilia in cellular mechanotransduction, the inventors hypothesized that increasing their length would enhance mechanosensitivity. Here, the inventors treated osteocytes with lithium and fenoldopam to increase ciliary length, and then mechanically stimulate the cells (Kathem et al., 2014; Miyoshi et al., 2009; Upadhyay et al., 2014). The results highlight the importance of cilium length in cellular mechanosensitivity, and that this is a process that can be modulated by pharmacologic intervention.

### (1) MODULATION OF PRIMARY CILIA LENGTH

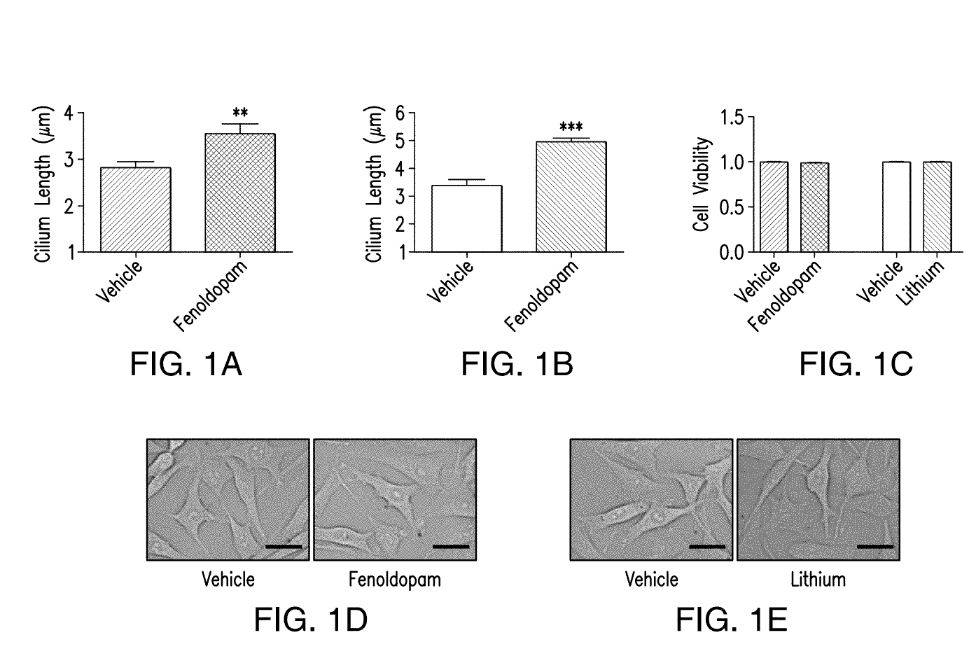

To test the hypothesis that cilium length directly affects mechanosensitivity, the inventors first verified that primary cilia length could be modulated. MLO-Y4 osteocytes were cultured and treated with two distinct small molecules to increase cilia length. Cells were cultured in media supplemented with fenoldopam, lithium, or vehicle control for 16 hours, and no gross morphological changes resulted from the drug treatments. Immunocytochemistry was then used to image primary cilia. Both fenoldopam and lithium treatments induced significant increases in cilia length by 26%±7% and 46%±5%, respectively, compared to vehicle control (FIG. 1A, B). Cell viability was assessed with MTT assay and it found no change upon drug treatments (FIG. 1C). Additionally, no gross morphological changes resulted from fenoldopam or lithium treatment (FIG. 1D, E).

### (2) EFFECT OF ELONGATING CILIA ON CELLULAR MECHANOSENSITIVITY

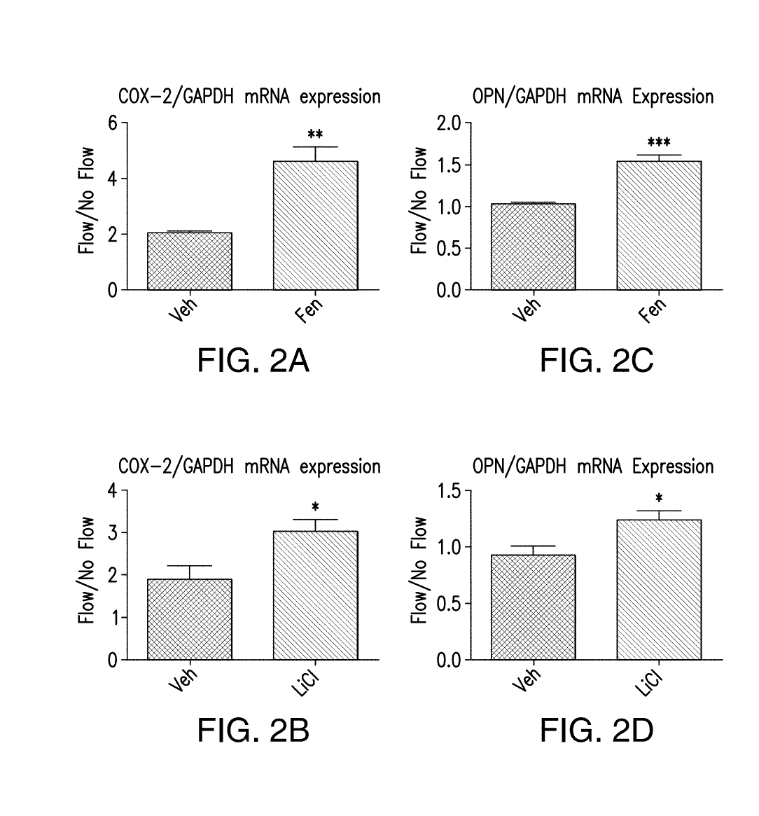

Next, the inventors examined the effect of elongating cilia on cellular mechanosensitivity by mechanically stimulating cells with longer cilia and analyzing their osteogenic response. Osteocytes were treated with fenoldopam, lithium, or vehicle control, and exposed to oscillatory fluid flow for 1 hr. As a control, samples were simultaneously loaded into flow chambers, but not subjected to flow. Mechanosensitivity was then quantified at the mRNA level with analysis of COX-2 and OPN expression, and presented as the fold change of flow over no flow control (FIG. 2). Cells with cilia lengthened by fenoldopam were more responsive, exhibiting elevated mRNA expression of 124%±27% and 48%±8% of COX-2 and OPN respectively compared to unlengthened controls. Lithium resulted in a more modest, but still significant increase in response of 61%±13% and 34%±8% for COX-2 and OPN. This flow-induced enhanced osteogenic response was observed in cells with elongated primary cilia, regardless of the means of lengthening, suggesting that the effect was due to lengthening and not an unanticipated effect of the agents utilized.

### (3) EFFECT OF PRIMARY CILIA LENGTH TO CILIA FUNCTION

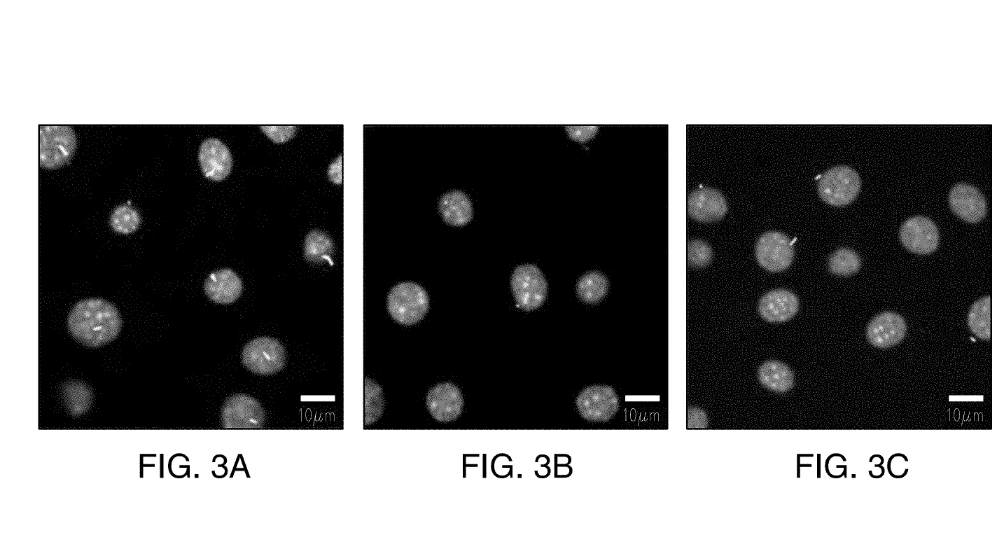

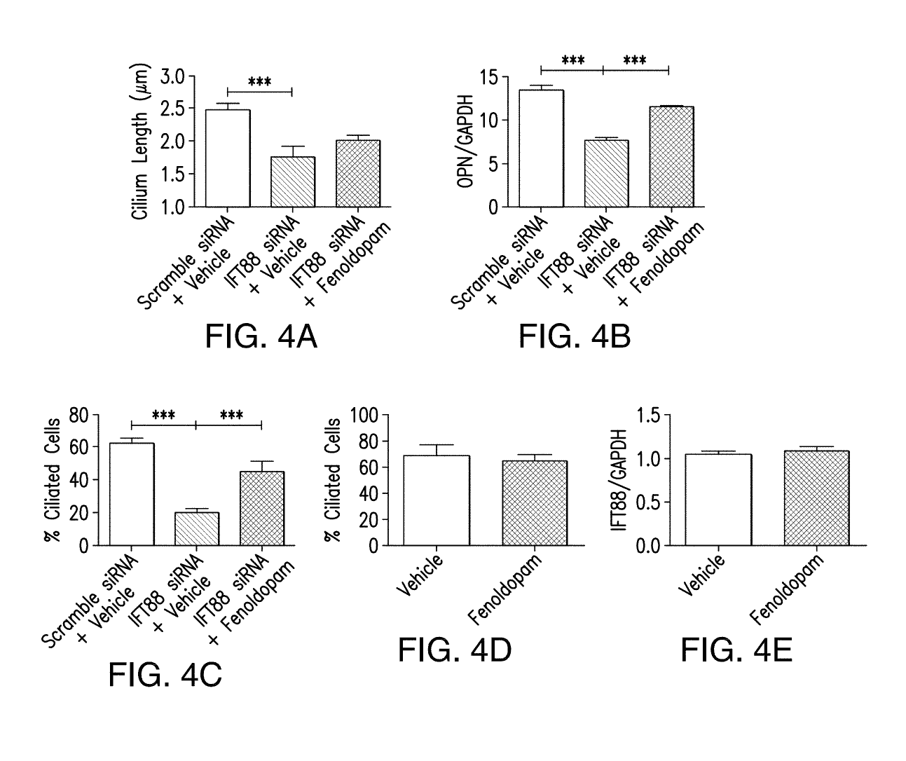

The inventors next sought to examine the potential of targeting primary cilia length to recover impaired cilia function. IFT88 inhibition was employed as a model of dysfunctional cilia, and has previously been used to mimic the effects of polycystic kidney disease (Lehman et al., 2008). IFT88 is a critical component of intraflagellar transport and is necessary for proper primary cilia formation (Pazour et al., 2000; Yoder et al., 2002). Cells treated with IFT88 siRNA displayed decreased primary cilia length and incidence compared to scramble control (FIG. 3A, B). IFT88 siRNA treated cells were then treated with fenoldopam, and cilia length and incidence were noticeably recovered (FIG. 3C). Upon analysis, cilia of IFT88 siRNA treated cells were significantly shorter (FIG. 4A) and were present with lower incidence (FIG. 4C) than scramble control groups, with fenoldopam treatment significantly recovering cilia incidence. Then the cells were mechanically stimulated to examine whether ciliogenesis recovery restored mechanosensitivity. Fluid flow was applied for 1 h and cells with impaired primary cilia formation displayed significantly decreased flow-induced OPN mRNA expression by 43%±2%, compared to scramble control (FIG. 4B). Then, fenoldopam treatment was able to recover this OPN response by 52%±1%, compared to IFT88 siRNA and vehicle treated cells. This further suggests that the length of primary cilia is critical to their function as a mechanosensor and that as cilia formation was restored, so was mechanosensitivity too. Then it was confirmed that fenoldopam treatment of healthy, ciliated cells had no significant effect on cilia incidence (FIG. 4D) or IFT88 mRNA expression (FIG. 4E).

### (4) MOLECULAR PATHWAY THROUGH WHICH FENOLDOPAM INCREASES PRIMARY CILIA LENGTH

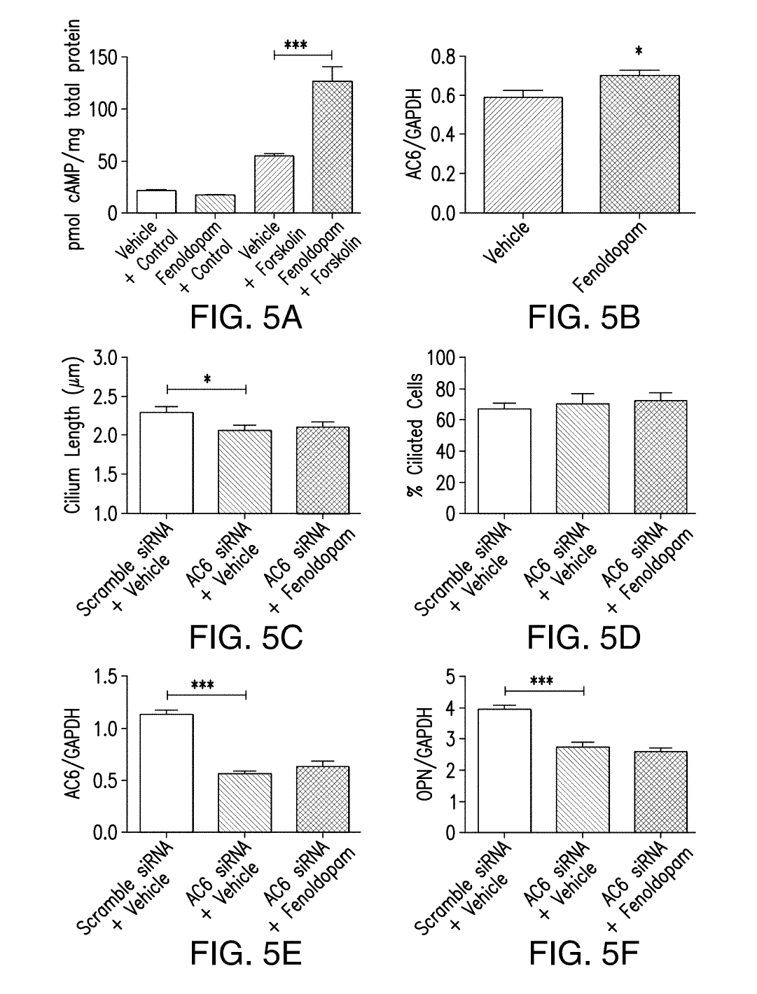

Finally, the inventors examined a potential molecular pathway through which fenoldopam increases primary cilia length. cAMP has been previously shown to be involved in ciliogenesis and primary cilia-mediated mechanotransduction, so adenylyl cyclase activity was quantified by measuring stimulated cAMP production (Besschetnova et al., 2010; Kwon et al., 2010). The inventors increased primary cilia length by fenoldopam treatment, and then briefly stimulated the cells with the adenylyl cyclase agonist, forskolin (FIG. 5A). Fenoldopam treatment significantly enhanced the forskolin stimulated cAMP response by 130%±25% compared to vehicle control. Additionally, fenoldopam treatment stimulated a 20%±8% increase in AC6 mRNA expression (FIG. 5B). Because of the significant role of AC6 in primary cilia-mediated mechanotransduction, inhibition of AC6 was used as an alternative model of impaired cell mechanosensitivity. Treatment of osteocytes with AC6 siRNA resulted in a small but significant decrease in cilia length by 10%±3% (FIG. 5C), while AC6 inhibition had no effect on cilia incidence (FIG. 5D). Fenoldopam had no effect on recovering cilia length or incidence in AC6 siRNA treated cells. When cells with diminished AC6 were mechanically stimulated, AC6 knockdown cells displayed decreased AC6 and flow-induced OPN expression by 50%±3% and 30%±3%, respectively, which was not recovered with fenoldopam treatment (FIG. 5E, F).

### (5) MATERIALS AND METHODS

(5-1) Cell Culture and Drug Treatments

MLO-Y4 osteocytes were cultured on collagen-coated dishes in MEMα (Life Tech) supplemented with 5% fetal bovine serum, 5% calf serum, and 1% penicillin/streptomycin at 37° C. and 5% CO2. Fenoldopam mesylate (Sigma) was used at 10 μM diluted in DMSO, dimethyl sulfoxide, (Sigma) and normal culture media. Lithium chloride (Sigma) was used at 500 μM diluted in normal culture media—a dose response from 50 μM to 10 mM was examined with 500 μM being the lowest dose to increase length significantly (data not shown). These agents, or their vehicle control, were applied to cells for 16 h prior to experimentation. MTT, (methylthiazolyldiphenyl-tetrazolium bromide) assay (Sigma) was performed according to manufacturer's protocol to assess cell viability during drug treatments. Phase contrast microscopy with an Olympus CKX41 inverted microscope and 40× objective was used to assess cell morphology.

(5-2) Immunocytochemistry

For primary cilia imaging and analysis, cells cultured on collagen I-coated glass were fixed in 10% formalin and treated with anti-acetylated α-tubulin primary antibody, 1:1, from a C3B9 hybridoma cell line (Sigma). Cilia were visualised with Alexa-Fluor 488 secondary antibody, 1:1000 (Life Technologies) and imaged with a 100× oil objective on an Olympus Fluoview FV1000 confocal microscope. Nuclei were stained with DAPI (Life Technologies). Cilia length was analyzed using Image J software.

(5-3) Oscillatory Fluid Flow

Cells were exposed to oscillatory fluid flow as a mechanical stimulus. Cells were seeded on collagen I-coated glass slides at ˜2,800 cells/cm² and cultured for 72 h before application of flow. Drug treatments were applied 16 h prior to experimentation. Slides were loaded into parallel plate flow chambers (dimensions: 75×38×0.28 mm) and allowed to incubate at 37° C. for 30 min prior to initiation of stimulation (Kwon et al., 2010; Lee et al., 2014; Malone et al., 2007). Flow was applied for 1 h at 1 Hz with a peak flow rate of 18.8 mL/min, providing 1 Pa peak wall shear stress.

(5-4) mRNA Expression

Immediately after flow, cells were washed with PBS and total mRNA was isolated using TriReagent (Sigma). Total mRNA was converted to cDNA by TaqMan reverse transcriptase (Applied Biosystems). Gene expression was analyzed by quantitative real-time PCR using primers and probes (Life Technologies) for analysis of cyclooxygenase-2, COX-2 (Mm00478374_m1); osteopontin, OPN (Mm00436767_m1); adenylyl cyclase 6, AC6 (Mm00475772_m1); intraflagellar transport 88, IFT88 (Mm00493675_m1); and GAPDH (4351309). Samples and standards were run in triplicate, and all gene expression was normalized to GAPDH endogenous control.

(5-5) RNA Interference

Gene silencing was performed by siRNA mediated knockdown and compared to scramble siRNA control (Life Technologies). For primary cilia disruption, cells were transfected with 20 μM IFT88 siRNA (5′-CCAGAAACAGATGAGGACGACCTTT-3′) (SEQ ID NO:1), AC6 siRNA (5′-CCTGCCACCTACAACAGCTCAATTA-3′) (SEQ ID NO:2), or scrambled siRNA control using Lipofectamine 2000 (Life Technologies) as previously described (Kwon et al., 2010).

(5-6) Adenylyl Cyclase Activity

Adenylyl cyclase activity was quantified by cAMP ELISA (Enzo). Cells were cultured as previously described and treated with 10 μM fenoldopam for 16 hours. Cells were stimulated by 10 μM forskolin (Sigma) or DMSO vehicle control for 20 minutes prior to lysis with 0.1 M HCl. Cell lysate was analyzed according to manufacturer's protocol, and normalized to total protein quantified by BCA (Thermo Fisher). All samples and standards were run in duplicate.

(5-7) Statistic Analysis

All data were analyzed with one-way ANOVA followed by Bonferroni post-hoc correction. Values are reported as mean±SEM, with p<0.05 considered statistically significant. Sample size, n, represents biological replicates.

### (6) DISCUSSION

The results demonstrated that primary cilia length plays a significant role in cell mechanosensitivity. Two distinct, clinically utilized, small molecules were employed to increase cilia length and both resulted in enhanced mechanosensitivity. Cells with impaired ciliogenesis had impaired mechanosensing, but this could be recovered with fenoldopam treatment. Finally, The results showed that fenoldopam modulates osteocyte mechanosensitivity through a mechanism involving AC6 and cells with diminished AC6 had shorter cilia and impaired mechanosensing.

Based on clinical and biochemical considerations, fenoldopam was a more suitable candidate for further study. Fenoldopam is a dopamine D1-like receptor agonist clinically used as a vasodilator in cases of extreme hypertension (Murphy et al., 2001; Post and Frishman, 1998). Lithium has a much less defined function and is clinically used to treat a wide range of mental disorders, including bipolar disorder (Marmol, 2008). Furthermore, lithium is an inhibitor of GSK-3β and can have downstream effects on various signaling pathways including Wnt and Hedgehog. While lithium has been used to increase cilia length in a variety of cell types, other GSK-3β inhibitors have no effect on cilia length (Jope, 2003; Ou et al., 2009). This suggests that lithium has off-target effects beyond modulating cilium length. Thus only fenoldopam was studied further as a cilium lengthening agent.

Fenoldopam treatment increased cilia length, but also plays a role in adenylyl cyclase activity. The increase in forskolin stimulated adenylyl cyclase activity with fenoldopam treatment implicates two potential mechanisms. First, it is possible that fenoldopam sensitizes adenylyl cyclases, resulting in an increased cAMP response to forskolin. Alternatively, fenoldopam may increase production of adenylyl cyclases, augmenting forskolin stimulated cAMP production. This second notion is consistent with previous work indicating that fenoldopam treatment upregulates AC6, a specific adenylyl cyclase isoform, production in kidney cells (Yu et al., 2014). The results support this possible molecular pathway, demonstrating an increase in AC6 mRNA expression in response to fenoldopam stimulation. Previously, the inventors have demonstrated that AC6 localizes to the osteocyte primary cilium and is critical for primary cilia-mediated mechanotransduction (Kwon et al., 2010). Besschetnova et al, reported that stimulating the cAMP signaling pathway results in increased cilia length (Besschetnova et al., 2010). Using an siRNA mediated knockdown, they then showed that AC6 has a functional role in mediating primary cilium elongation.

Adenylyl cyclases and cAMP contribute to recovering and elongating primary cilia by stimulating IFT particle transport. It has previously been reported that stimulation of the adenylyl cyclase-cAMP-PKA signaling pathway augments anterograde transport of IFT particles to promote cilium elongation (Besschetnova et al., 2010). Because fenoldopam enhances adenylyl cyclase production, this suggests that fenoldopam treatment is potentiating adenylyl cyclase activity and IFT particle transport. The model of impaired cilia utilized an IFT88 knockdown, not a complete knockout of the gene, so it is possible that fenoldopam was able to enhance remaining IFT88 function and promote cilium elongation. Furthermore, this presupposes that even though the IFT88 knockdown is satisfactory to impair cilia formation and function, sufficient IFT88 remains to elongate cilia. The data show no change in IFT88 mRNA expression elicited by fenoldopam treatment suggesting that fenoldopam stimulated the remaining IFT88, rather than promoting production of new IFT88. This does not, however, discount the notion that fenoldopam treatment may instead prevent IFT88 knockdown driven disassembly of the cilium.

The specific means by which cells with longer cilia are more mechanosensitive remains elusive, but there are two potential mechanisms of how this may occur. Schwartz et al, developed one of the first models of primary cilia deflection under fluid flow and hypothesized that longer cilia would experience greater membrane strain to increase opening of stretch-activated ion channels on the ciliary membrane (Schwartz et al., 1997). Alternatively, longer cilia may simply allow for the presence of more cilia-specific proteins and signaling molecules within this microdomain (Breslow et al., 2013; Kee et al., 2012). Increasing the total amount of ciliary protein could enhance signal transduction within the ciliary compartment, modifying primary cilia-mediated mechanosensitivity. In fact, fenoldopam treated cells exposed to fluid flow have increased ciliary influx of calcium, which has been identified as one initiator of the mechanotransduction signaling cascade (Jin et al., 2014; Yuan et al., 2015). It is also possible that cilium-lengthening agents actually enhance ciliary protein production and trafficking to promote cilium elongation.

The correlation between cilia length and critical ciliary proteins involved in mechanosensing was examined with AC6 siRNA treatment. The knockdown of AC6 decreased flow-induced osteogenic gene expression, yet fenoldopam treatment was not sufficient to rescue AC6 expression or OPN expression as was demonstrated in the IFT88 knockdown model. Because fenoldopam treatment was not able to recover AC6 or OPN mRNA expression in AC6 knockdown cells, this may suggest that the ability of fenoldopam to enhance AC6 activity is critical for recovering cellular mechanosensing. However, AC6 knockdown also decreased primary cilia length, which was not recovered with fenoldopam and did not alter cilia incidence. Altogether, these data suggest that both cilia length and protein production may be critical in primary cilia-mediated mechanosensing.

Cells with longer cilia are more mechanosensitive, but primary cilia cannot be elongated indefinitely. Longer cilia are exposed to greater drag force, and are more likely to be sheared off (Hierck et al., 2008). For example, endothelial cell primary cilia are flow sensors in regions of low shear specifically because they are cleaved off as shear stresses increase (Van der Heiden et al., 2008). Interestingly, electron microscopy has shown that primary cilia structure is not constant along the ciliary axoneme and becomes increasingly disorganized and asymmetric at the distal tip (Odor and Blandau, 1985; Yamamoto and Kataoka, 1986). This loss of microtubule symmetry reduces the bending stiffness of the cilium at the distal end, making drastically elongated cilia more susceptible to removal by fluid shear (Rydholm et al., 2010).

While osteocyte primary cilia are free-standing flow sensors in vitro, their mechanosensing function may differ in vivo. It has been estimated that the lacunar space in which osteocytes reside in vivo allows for only a 1 μm long cilium (McNamara et al., 2009; Uzbekov et al., 2012). Due to the spatial limitations within the lacuna, the potential effect of pharmacologically enhancing osteocyte cilia length in vivo is unclear. In fact, these spatial constraints may point to the cilium not being a free-flowing mechanosensor at all. Rather, osteocyte cilia may anchor to the lacunar wall, similarly to chondrocyte primary cilia which form integrin attachments with the surrounding extracellular matrix, ECM (McGlashan et al., 2006). A computational model by Vaughan et al, simulated osteocytes exposed to fluid flow within the lacunar-canalicular network (Vaughan et al., 2014). The authors modeled a free-standing cilium, 0.5 μm long, within a lacuna and calculated the resulting strain at the base of the cilium. Their model suggests that a cilium in this configuration does not experience a great enough strain to function as a flow sensor, but a cilium directly attached to the ECM does. The authors, however, do not account for the amount of membrane strain necessary to stimulate stretch-activated ion channels on the ciliary membrane, and may have overestimated the required strain for cilium stimulation. Fenoldopam treatment not only increases length, but may also enhance mechanosensitive protein levels, such as adenylyl cyclases and ion channels, within the cilium. Regardless of cilium length, this enriched protein trafficking to the cilium would increase chemical kinetics within the ciliary microdomain to modify cellular mechanosensitivity.

Targeting primary cilia-mediated mechanotransduction has widespread applications in preventative medicine that reach far beyond osteocytes. Numerous diseases are characterized by impaired primary cilia function. Mutations of PC2, polycystin 2, are attributed to polycystic kidney disease and skeletal deformations. Bardet-Biedl syndrome is characterized by malfunctioning BBS proteins at the base of the primary cilium, causing retinopathy, polydactyly, and renal failure (Loktev et al., 2008; Mochizuki et al., 1996; Xiao et al., 2011). Recently, primary cilia have even been implicated in tumor development. Primary cilia help regulate Wnt signaling, changes in which have been correlated with cancer cell progression (Lancaster et al., 2011). Furthermore, some cancer cell types lose their primary cilia, which potentially contributes to their insensitivity to repressive signals (Plotnikova et al., 2008). Additionally, atherosclerotic plaques form in areas of low and disturbed arterial fluid flow, regions that interestingly have an increased incidence of primary cilia. This suggests that these cells are compensating, increasing their sensitivity to low fluid flow in order to promote an adequate cellular response (Van der Heiden et al., 2008; Warboys et al., 2011). Within bone, osteocytes utilize primary cilia to sense and respond to mechanical cues. In vitro and in vivo studies demonstrate that when these cilia are removed there is a decreased bone formation response to loading (Kwon et al., 2010; Malone et al., 2007; Temiyasathit et al., 2012). Fenoldopam is already an FDA approved drug, and The results point to it being an attractive candidate for study in numerous in vitro and in vivo applications to treat such a myriad of conditions.

### (7) REFERENCES

Besschetnova T Y, Kolpakova-Hart E, Guan Y, Zhou J, Olsen B R, Shah J V (2010) Identification of signaling pathways regulating primary cilium length and flow-mediated adaptation. Curr. Biol. 20: 182-7. doi:10.1016/j.cub.2009.11.072.
Breslow D K, Koslover E F, Seydel F, Spakowitz A J, Nachury M V (2013) An in vitro assay for entry into cilia reveals unique properties of the soluble diffusion barrier. J. Cell Biol. 203: 129-47. doi:10.1083/jcb.201212024.
Ehrlich P, Lanyon L (2002) Mechanical strain and bone cell function: a review. Osteoporos. Int.: 688-700. doi:10.1007/s001980200095.
Fujihara S, Yokozeki M, Oba Y, Higashibata Y, Nomura S, Moriyama K (2006) Function and regulation of osteopontin in response to mechanical stress. J Bone Min. Res 21: 956-964. doi:10.1359/jbmr.060315.
Van der Heiden K, Hierck B P, Krams R, de Crom R, Cheng C, Baiker M, Pourquie MJBM, Alkemade F E, DeRuiter M C, Gittenberger-de Groot A C, Poelmann R E (2008) Endothelial primary cilia in areas of disturbed flow are at the base of atherosclerosis. Atherosclerosis 196: 542-50. doi:10.1016/j.atherosclerosis.2007.05.030.
Hierck B P, Van der Heiden K, Alkemade F E, Van de Pas S, Van Thienen J V, Groenendijk B C, Bax W H, Van der Laarse A, Deruiter M C, Horrevoets A J, Poelmann R E (2008) Primary cilia sensitize endothelial cells for fluid shear stress. Dev Dyn 237: 725-735. doi:10.1002/dvdy.21472.
Jin X, Mohieldin A M, Muntean B S, Green J a, Shah J V, Mykytyn K, Nauli S M (2014) Cilioplasm is a cellular compartment for calcium signaling in response to mechanical and chemical stimuli. Cell. Mol. Life Sci. 71: 2165-78. doi:10.1007/s00018-013-1483-1.
Jope R S (2003) Lithium and GSK-3: One inhibitor, two inhibitory actions, multiple outcomes. Trends Pharmacol. Sci. 24: 441-443. doi:10.1016/S0165-6147(03)00206-2.
Kathem S H, Mohieldin A M, Abdul-Majeed S, Ismail S H, Altaei Q H, Alshimmari I K, Alsaidi M M, Khammas H, Nauli A M, Joe B, Nauli S M (2014) Ciliotherapy: a novel intervention in polycystic kidney disease. J. Geriatr. Cardiol. 11: 63-73. doi:10.3969/j.issn.1671-5411.2014.01.001.
Kee H L, Dishinger J F, Lynne Blasius T, Liu C-J, Margolis B, Verhey K J (2012) A size-exclusion permeability barrier and nucleoporins characterize a ciliary pore complex that regulates transport into cilia. Nat. Cell Biol. doi:10.1038/ncb2450.
Klein-Nulend J, Burger E H, Semeins C M, Raisz L G, Pilbeam C C (1997) Pulsating fluid flow stimulates prostaglandin release and inducible prostaglandin G/H synthase mRNA expression in primary mouse bone cells. J. Bone Miner. Res. 12: 45-51. doi:10.1359/jbmr.1997.12.1.45.
Ku D N (1997) Blood Flow in Arteries. Annu. Rev. Fluid Mech. 29: 399-434. doi:10.1146/annurev.fluid.29.1.399.
Kwon R Y, Temiyasathit S, Tummala P, Quah C C, Jacobs C R (2010) Primary cilium-dependent mechanosensing is mediated by adenylyl cyclase 6 and cyclic AMP in bone cells. FASEB J. 24: 2859-68. doi:10.1096/fj.09-148007.
Lancaster M a, Schroth J, Gleeson J G (2011) Subcellular spatial regulation of canonical Wnt signalling at the primary cilium. Nat. Cell Biol. 13: 700-7. doi:10.1038/ncb2259.
Lee K L, Hoey D A, Spasic M, Tang T, Hammond H K, Jacobs C R (2014) Adenylyl cyclase 6 mediates loading-induced bone adaptation in vivo. FASEB J. 28: 1157-1165. doi:10.1096/fj.13-240432.
Lehman J M, Michaud E J, Schoeb T R, Aydin-Son Y, Miller M, Yoder B K (2008) The Oak Ridge Polycystic Kidney mouse: Modeling ciliopathies of mice and men. Dev. Dyn. 237: 1960-1971. doi:10.1002/dvdy.21515.
Liu W, Xu S, Woda C, Kim P, Weinbaum S, Satlin L M (2003) Effect of flow and stretch on the [Ca2+]i response of principal and intercalated cells in cortical collecting duct. Am. J. Physiol. Renal Physiol. 285: F998-F1012. doi:10.1152/ajprenal.00067.2003.
Loktev A V, Zhang Q, Beck J S, Searby C C, Scheetz T E, Bazan J F, Slusarski D C, Sheffield V C, Jackson P K, Nachury M V (2008) A BBSome Subunit Links Ciliogenesis, Microtubule Stability, and Acetylation. Dev. Cell 15: 854-865. doi:10.1016/j.devcel.2008.11.001.
Malone A M D, Anderson C T, Tummala P, Kwon R Y, Johnston T R, Stearns T, Jacobs C R (2007) Primary cilia mediate mechanosensing in bone cells by a calcium-independent mechanism. Proc. Natl. Acad. Sci. U.S.A 104: 13325-13330. doi:10.1073/pnas.0700636104.
Marmol F (2008) Lithium: Bipolar disorder and neurodegenerative diseases Possible cellular mechanisms of the therapeutic effects of lithium. Prog. Neuro-Psychopharmacology Biol. Psychiatry 32: 1761-1771. doi:10.1016/j.pnpbp.2008.08.012.
McGlashan S R, Jensen C G, Poole C A (2006) Localization of extracellular matrix receptors on the chondrocyte primary cilium. J. Histochem. Cytochem. 54: 1005-1014. doi:10.1369/jhc.5A6866.2006.
McNamara L M, Majeska R J, Weinbaum S, Friedrich V, Schaffler M B (2009) Attachment of osteocyte cell processes to the bone matrix. Anat Rec 292: 355-363. doi:10.1002/ar.20869.
Miyoshi K, Kasahara K, Miyazaki I, Asanuma M (2009) Lithium treatment elongates primary cilia in the mouse brain and in cultured cells. Biochem. Biophys. Res. Commun. 388: 757-62. doi:10.1016/j.bbrc.2009.08.099.
Mochizuki T, Wu G, Hayashi T, Xenophontos S L, Veldhuisen B, Saris J J, Reynolds D M, Cai Y, Gabow P A, Pierides A, Kimberling W J, Breuning M H, Deltas C C, Peters D J, Somlo S (1996) PKD2, a gene for polycystic kidney disease that encodes an integral membrane protein. Science 272: 1339-1342. doi:10.1126/science.272.5266.1339.
Murphy M B, Murray C, Shorten G D (2001) Fenoldopam: a selective peripheral dopamine-receptor agonist for the treatment of severe hypertension. N. Engl. J. Med. 345: 1548-1557. doi:10.1056/NEJMra010253.
Odor D L, Blandau R J (1985) Observations on the solitary cilium of rabbit oviductal epithelium: its motility and ultrastructure. Am. J. Anat. 174: 437-453. doi:10.1002/aja.1001740407.
Ou Y, Ruan Y, Cheng M, Moser J J, Rattner J B, van der Hoorn F a. (2009) Adenylate cyclase regulates elongation of mammalian primary cilia. Exp. Cell Res. 315: 2802-2817. doi:10.1016/j.yexcr.2009.06.028.
Pazour G J, Dickert B L, Vucica Y, Seeley E S, Rosenbaum J L, Witman G B, Cole D G (2000) Chlamydomonas IFT88 and its mouse homologue, polycystic kidney disease gene Tg737, are required for assembly of cilia and flagella. J. Cell Biol. 151: 709-718. doi:10.1083/jcb.151.3.709.
Plotnikova O V, Golemis E a, Pugacheva E N (2008) Cell cycle-dependent ciliogenesis and cancer. Cancer Res. 68: 2058-61. doi:10.1158/0008-5472.CAN-07-5838.
Post J B, Frishman W H (1998) Fenoldopam: a new dopamine agonist for the treatment of hypertensive urgencies and emergencies. J. Clin. Pharmacol. 38: 2-13.
Praetorius H A, Spring K R (2001) Bending the MDCK cell primary cilium increases intracellular calcium. J. Membr. Biol. 184: 71-79. doi:10.1007/s00232-001-0075-4.
Praetorius H A, Spring K R (2003) Removal of the MDCK cell primary cilium abolishes flow sensing. J. Membr. Biol. 191: 69-76. doi:10.1007/s00232-002-1042-4.
Raisz L G (1999) Prostaglandins and bone: Physiology and pathophysiology. Osteoarthr. Cartil. 7: 419-421. doi:10.1053/joca.1998.0230.
Rydholm S, Zwartz G, Kowalewski J M, Kamali-Zare P, Frisk T, Brismar H (2010) Mechanical properties of primary cilia regulate the response to fluid flow. Am. J. Physiol. Renal Physiol. 298: F1096-102. doi:10.1152/ajprenal.00657.2009.
Schwartz E A, Leonard M L, Bizios R, Bowser S S (1997) Analysis and modeling of the primary cilium bending response to fluid shear. Am. J. Physiol. 272: F132-F138.
Temiyasathit S, Tang W J, Leucht P, Anderson C T, Monica S D, Castillo A B, Helms J A, Stearns T, Jacobs C R (2012) Mechanosensing by the primary cilium: Deletion of Kif3a reduces bone formation due to loading. PLoS One 7. doi:10.1371/journal.pone.0033368.
Upadhyay V S, Muntean B S, Kathem S H, Hwang J J, Aboualaiwi W a, Nauli S M (2014) Roles of dopamine receptor on chemosensory and mechanosensory primary cilia in renal epithelial cells. Front. Physiol. 5: 72. doi:10.3389/fphys.2014.00072.
Uzbekov R E, Maurel D B, Aveline P C, Pallu S, Benhamou C L, Rochefort G Y (2012) Centrosome fine ultrastructure of the osteocyte mechanosensitive primary cilium. Microsc. Microanal. 18: 1430-41. doi:10.1017/S1431927612013281.
Vaughan T J, Mullen C a, Verbruggen S W, McNamara L M (2014) Bone cell mechanosensation of fluid flow stimulation: a fluid-structure interaction model characterising the role integrin attachments and primary cilia. Biomech. Model. Mechanobiol. 2. doi:10.1007/s10237-014-0631-3.
Warboys C M, Amini N, de Luca A, Evans P C (2011) The role of blood flow in determining the sites of atherosclerotic plaques. F1000 Med. Rep. 3: 5. doi:10.3410/M3-5.
Xiao Z, Dallas M, Qiu N, Nicolella D, Cao L, Johnson M, Bonewald L, Quarles L D (2011) Conditional deletion of Pkd1 in osteocytes disrupts skeletal mechanosensing in mice. FASEB J. 25: 2418-2432. doi:10.1096/fj.10-180299.
Yamamoto M, Kataoka K (1986) Electron microscopic observation of the primary cilium in the pancreatic islets. Arch. Histol. Jpn. Nippon soshikigaku kiroku 49: 449-457.
Yoder B K, Tousson A, Millican L, Wu J H, Bugg C E, Schafer J A, Balkovetz D F (2002) Polaris, a protein disrupted in orpk mutant mice, is required for assembly of renal cilium. Am. J. Physiol. Renal Physiol. 282: F541-F552. doi:10.1152/ajprenal.00273.2001.
You L, Temiyasathit S, Lee P, Kim C H, Tummala P, Yao W, Kingery W, Malone A M, Kwon R Y, Jacobs C R (2008) Osteocytes as mechanosensors in the inhibition of bone resorption due to mechanical loading. Bone 42: 172-179. doi:10.1016/j.bone.2007.09.047.
Yu P, Sun M, Villar V A M, Zhang Y, Weinman E J, Felder R a, Jose P a (2014) Differential dopamine receptor subtype regulation of adenylyl cyclases in lipid rafts in human embryonic kidney and renal proximal tubule cells. Cell. Signal. 26: 2521-2529. doi:10.1016/j.cellsig.2014.07.003.
Yuan S, Zhao L, Brueckner M, Sun Z (2015) Intraciliary Calcium Oscillations Initiate Vertebrate Left-Right Asymmetry. Curr. Biol. 25: 556-567. doi:10.1016/j.cub.2014.12.051.

### Example 2: Primary Cilia-Mediated Mechanotransduction Regulates Osteocyte Paracrine Signaling to Osteoblasts

### (1) INTRODUCTION

Primary cilia, solitary non-motile antennae, mediate mechanosensing in numerous cell types, such as kidney, cartilage, and bone cells [1, 2, 3]. Impairing/removing primary cilia diminishes cell mechanosensitivity [3]. Stiffening primary cilia, by increasing microtubule acetylation, decreases mechanosensitivity [4]. Paracrine signals from mechanically stimulated osteocytes promote MSC osteogenic differentiation [5]. Flow-induced mechanical stimulation initiates mechanotransduction [6]. Mechanical load is a potent anabolic stimulus of bone formation [3]. The objective of this study is to determine whether the osteocyte cilium is involved in signaling to osteoblasts and how this signaling can be potentiated via ciliary targeted therapies

### (2) METHODS

Cell Culture

MLO-Y4 osteocytes were cultured on collagen coated dishes. MC3T3 osteoblasts were cultured on tissue culture dishes. Osteocyte media was supplemented with 10 μM fenoldopam for 16 hours prior to rocking, 5 μM tubastatin for 3 hours, or DMSO vehicle control.

In Vitro Mechanical Stimulation

Oscillatory fluid flow was applied to osteocytes at 0.12 Pa peak wall shear stress using a rocker plate set-up [8]. MLO-Y4s were subjected to 2, 6, 12, and 24 hours of flow. Cells not exposed to rocking were used as a no flow control. 12 hours of flow was then used for the remainder of studies. Osteocytes were subjected to drug treatment or siRNA-mediated knockdown prior to initiation of flow. Conditioned osteocyte media was used to culture osteoblasts for 24 hours before analysis.

In Vivo Drug Treatment and Loading

Skeletally mature, 16 week old, C57Bl/6 mice were treated with 20 mg/kg fenoldopam by subcutaneous injection on 7 consecutive days. Compressive ulnar loading was applied on days 5-7 to mechanically stimulate bone (3N at 2 Hz for 120 cycles) [9].

Primary Cilia Disruption

Osteocyte primary cilia formation was inhibited by siRNA-mediated knockdown of IFT88 [3, 5]. AC6, TRPV4, and PC2 were also inhibited by siRNA treatment [10].

Statistics

All data reported as mean±SEM, *p<0.05 **p<0.01, ***p<0.001.

### (3) RESULTS

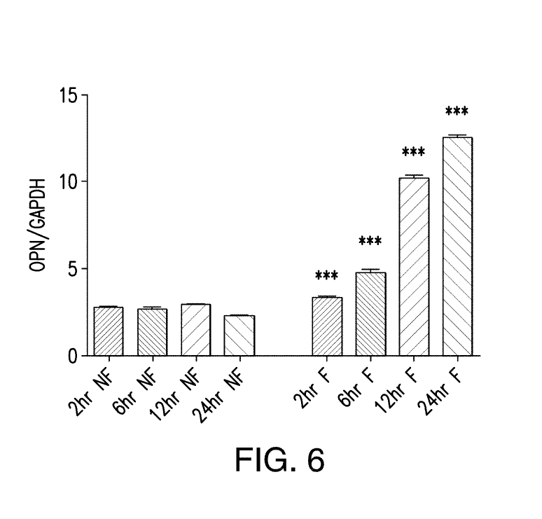

Osteoblast osteogenic gene expression was enhanced by culture with conditioned media from mechanically stimulated osteocytes (FIG. 6). Osteopontin (OPN) expression was normalized to GAPDH and compared to no flow controls.

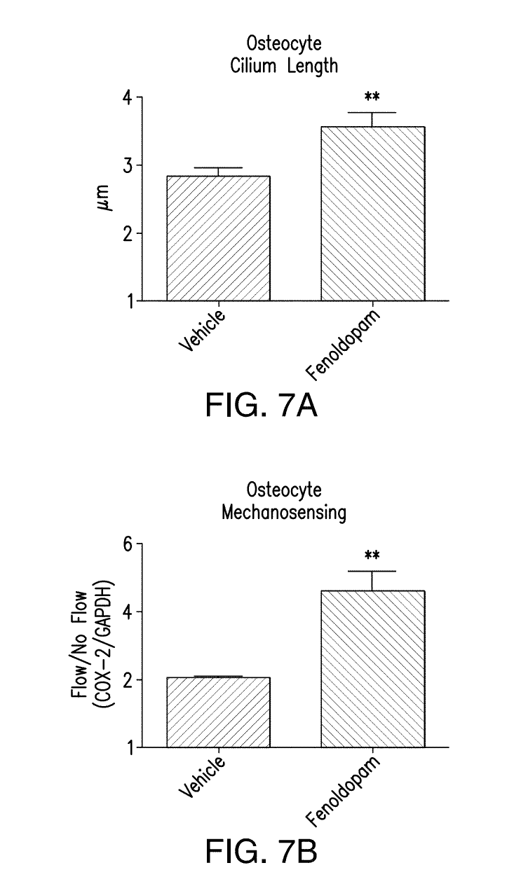

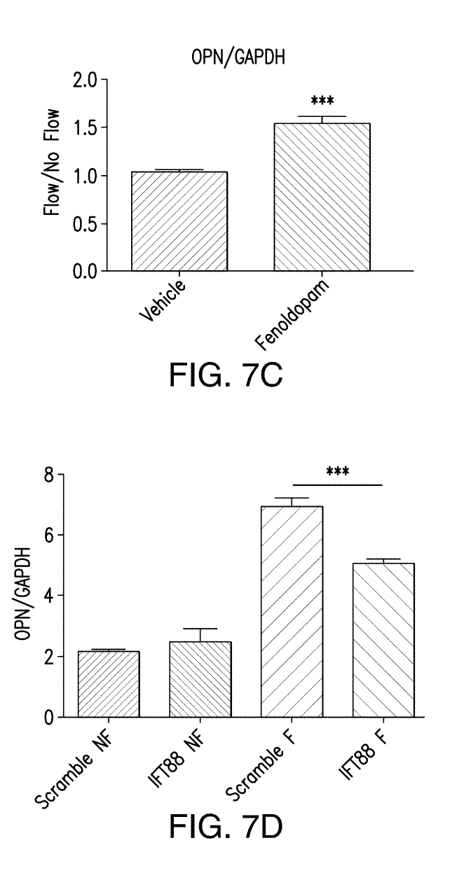

Osteocytes with longer primary cilia were more mechanosensitive. Fenoldopam treatment increased osteocyte primary cilia length, as well as the OPN and COX-2 response to flow. OPN and COX-2 expressions were normalized to GAPDH and compared to no flow controls (FIG. 7A-C). Osteoblast osteogenic gene expressions were diminished by disruption of osteocyte primary cilia formation (FIG. 7D). Primary cilia were impaired by siRNA-mediated KD of IFT88. Media from mechanically stimulated osteocytes with impaired cilia elicited an abrogated response in osteoblasts compared to scramble control.

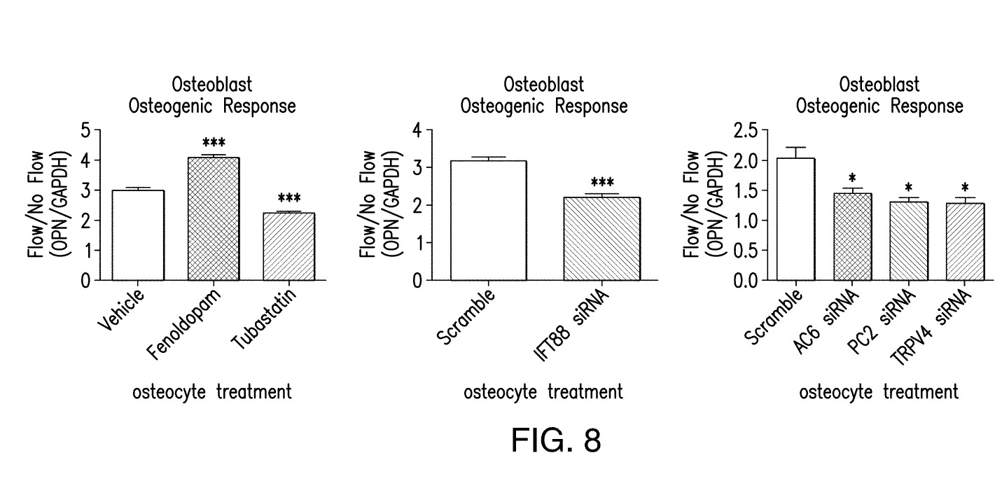

Osteocyte primary cilia directed osteogenic paracrine signaling. Osteoblast osteogenic response to paracrine signals from mechanically stimulated osteocytes was assessed by osteopontin, OPN, mRNA expression (FIG. 8). Osteocytes were treated with fenoldopam (increase cilia length and mechanosensing), or tubastatin (increase cilia stiffness to impair mechanosensing). Osteocyte cilia formation was also inhibited, IFT88 knockdown, as well as pools of key cilia mechanotransduction proteins—AC6, PC2, TRPV4 (FIG. 8).

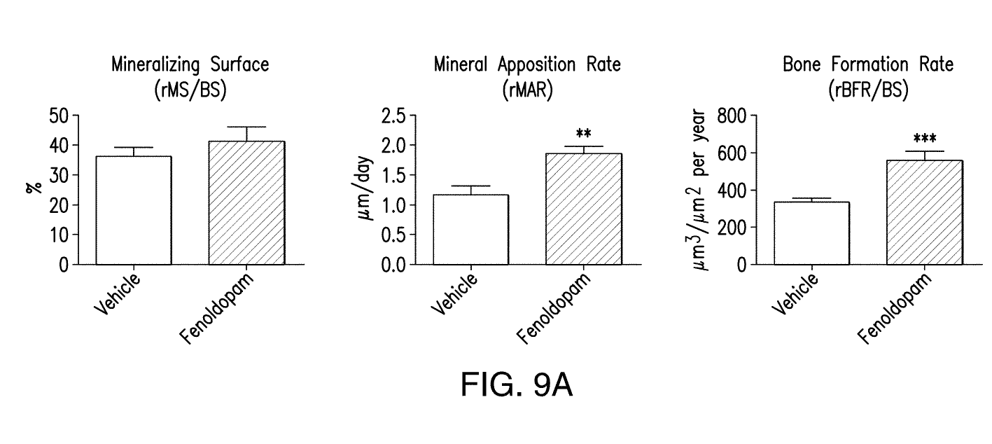

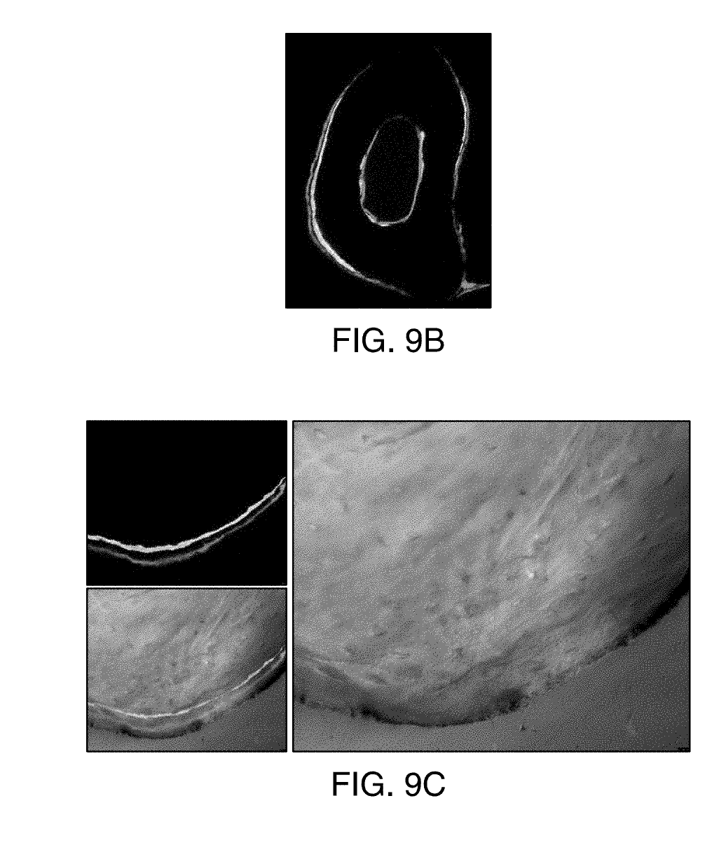

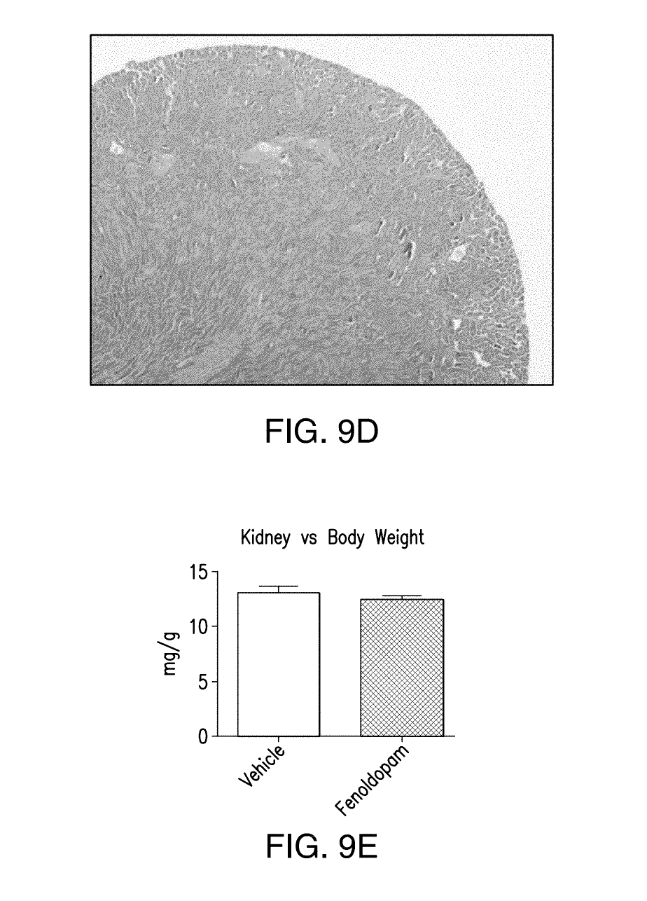

Fenoldopam treatment enhances load-induced bone formation (FIG. 9A). Skeletally mature mice were administered fenoldopam for 7 consecutive days. Compressive ulnar load was applied for 3 days to mechanically stimulate the bones, while contralateral limbs served as non-loaded controls. Dynamic histomorphometric analysis was performed to assess bone adaptation. The amount of mineralizing surface (rMS/BS) remained unchanged, while mineral apposition (rMAR) and bone formation (rBFR/BS) rates significantly increased with fenoldopam treatment (FIG. 9A). N≥12 for each group. 2-way ANOVA revealed no statistical difference based on gender.

Load-induced bone formation was assessed by dynamic histo-morphometry (FIG. 9B). Alizarin (red) was administered four days after calcein (green). Bone formation was measured at periosteal surface. Minimal adverse effects of drug treatment is shown in FIG. 9C-E. There is no difference in visible bone ultrastructure between fenoldopam and vehicle control mice (FIG. 9C). Mouse weight, kidney weight, and kidney morphology assessed by H&E stain, remained unchanged in drug vs vehicle control (FIG. 9D-E). μCT analysis also revealed no change in normal bone properties due to drug treatment.

TABLE 1
uCT analysis of ulnar midshaft shows no change in normal bone
architecture with drug treatment compared to control.

| | Female | | Male | |
|---|---|---|---|---|
| Bone and parameter | Vehicle | Fenoldopam | Vehicle | Fenoldopam |

Ulnar midshaft

| | | | | |
|---|---|---|---|---|
| n | 9 | 5 | 8 | 7 |
| Total area (mm²) | 0.352 ± 0.019 | 0.357 ± 0.005 | 0.380 ± 0.018 | 0.370 ± 0.027 |
| Cortical area (mm²) | 0.306 ± 0.017 | 0.310 ± 0.003 | 0.330 ± 0.016 | 0.320 ± 0.023 |
| Cortical | 0.173 ± 0.008 | 0.173 ± 0.006 | 0.172 ± 0.005 | 0.169 ± 0.009 |
| thickness (mm) | | | | |
| Iₘₐₓ (mm⁴) | 0.028 ± 0.004 | 0.029 ± 0.001 | 0.037 ± 0.006 | 0.033 ± 0.007 |
| Iₘᵢₙ (mm⁴) | 0.004 ± 0.001 | 0.005 ± 0.000 | 0.005 ± 0.001 | 0.004 ± 0.001 |
| Porosity (%) | 0.131 ± 0.005 | 0.131 ± 0.006 | 0.133 ± 0.004 | 0.134 ± 0.003 |
| Bone Mineral | 1236.399 ± 15.089 | 1224.143 ± 11.750 | 1234.686 ± 25.702 | 1218.543 ± 20.338 |
| Density (mg/mm³) | | | | |

### (4) DISCUSSION

Primary cilia disruption in mechanically stimulated osteocytes diminishes the osteogenic response in osteoblasts, demonstrating a role of primary cilia in osteocyte mechanotransduction, as well as downstream signaling to other cell types. Osteocytes are a paracrine signaling nexus that directs not only MSC differentiation, but also osteoblast activity Primary cilia disruption in osteocytes does not completely abolish the flow-induced OPN increase in osteoblasts, suggesting that other cellular mechanosensors, such as integrins or gap junctions, may also be involved. This paracrine signaling model does not discount the potential of intercellular signaling by cell-cell contact between mechanically stimulated osteocytes and osteoblasts. Pharmacologic manipulation of osteocyte cilia alters mechanotransduction response and paracrine signaling to osteoblasts.

Pharmacologically targeting the primary cilia apparatus can be a potential therapeutic strategy to promote bone formation. Treatment with a cilia lengthening agent can sensitize bone to mechanical stimulation. Increased mineral apposition and bone formation rates, with no change in amount of mineralizing surface, indicates increased osteoblast activity, consistent with in vitro results. Fenoldopam is a DR1 agonist clinically used to treat hypertension, but never used for any bone indication. No change in normal bone properties, animal weight, kidney weight, or kidney morphology suggests minimal adverse effects of drug treatment.

### (5) References

1. Praetorius et al. (2003) J Membr Biol. 191(1) 69-76.
2. McGlashan et al. (2010) Cell Biol Int. 34(5):441-6.
3. Malone et al. (2007) PNAS 104(33): 13325-13330.
4. Nguyen et al. (2015) Biol Open. 4(12):1733-8.
5. Hoey et al. (2011) Biochem Biophys Res Commun. 412(1):182-7.
6. Battle et al. (2015) PNAS 112(5):1410-5.
7. Duffy and Jacobs (2015) Biophys J 108(7):1583-4.
8. Zhou et al. (2010) J Biomech. 43(8):1598-602.
9. Lee et al. (2014) FASEBJ. 28(3):1157-65. [10] Lee et al. (2015) Cilia. 4:7.

### Example 3: Targeting the Mechanobiology of the Osteocyte Primary Cilium to Bias Bone Formation

### Introduction

The burden of osteoporosis and low bone mass is unrelenting, affecting over 50% of the US population over 50, and is compounded by the insufficiency of prophylaxis and treatment options. The inventors and others have established the osteocyte primary cilium—a mechanosensing antenna-like organelle—as a promising pharmaceutical target to exploit the natural anabolic response to physical loading to maintain bone health. Nonetheless, how key molecular components of the osteocyte primary cilium microdomain can be manipulated to enhance bone formation without adversely impacting bone physiology remains a critical gap in knowledge.

The central hypothesis is that the unique mechanobiology of the osteocyte primary cilium can be targeted to bias bone formation while minimizing disruption to normal physiology. This is based on the rationale that the osteocyte primary cilium plays a key role in bone mechanotransduction and preliminary data suggest it can be selectively manipulated. Specifically agents that modify cilium structural properties (length and stiffness) and its osteogenic signaling modulate load-induced bone formation. Furthermore, preliminary evidence collected with the inventors' novel biosensors indicates that aspects of calcium/cAMP signaling dynamics within the ciliary microdomain are unique to osteocytes. Particularly, with a ciliary-localizing calcium biosensor, the inventors have identified TRPV4 as the principal mechanosensitive calcium ion channel. The inventors have also demonstrated that AC6 is an important adenylyl cyclase in osteocyte primary cilia and that mice lacking AC6 have an abrogated response to load-induced bone formation, similar to an osteocyte primary cilia knockout model. With the expanding biosensor capabilities, the inventors will define distinct molecular targets of the osteocyte cilium and employ a multifaceted approach designed to ensure that the proposed therapeutic strategy does not have off-target effects on intercellular signaling or remodeling.

The following specific aims will test the central hypothesis by establishing the potential of osteocyte cilia therapeutics in vivo (SA1), defining the osteocyte cilia microdomain signaling cascade to inform development of improved treatment strategies (SA2), and characterize the role of the osteocyte primary cilium in directing bone intercellular communication and physiology (SA3).

SA1: Therapeutically Manipulate the Physical and Biochemical Composition of the Primary Cilium Microdomain and Quantify the Resulting Change in Load-Induced Bone Formation In Vivo.

Working hypothesis: Biochemically potentiating the ciliary microdomain increases load-induced bone formation. Skeletally mature mice will be treated with agents that lengthen (fenoldopam) and stiffen (tubastatin) primary cilia. TRPV4 will be agonized (4αPDD, GSK101) and antagonized (RN1734). Load-induced bone formation will be quantified via dynamic histomorphometry, microCT analysis, and mechanical testing.

SA2: Characterize the Osteocyte Intraciliary Molecular Signaling Apparatus and Determine how its Dynamics are Affected by Altering Ciliary Structure and Composition.

Working Hypothesis: Calcium kinetics and cAMP signaling are coupled by adenylyl cyclases within the cilium and changes in their dynamics culminate in altered osteogenic gene expression. This aim relies on unique ciliary cAMP and calcium biosensors to measure calcium and cAMP within the ciliary microdomain. Coupling between these two second messengers will be confirmed by introducing a disruptive mutation to the AC6 calcium binding pocket. Targeting sequences will be altered to manipulate ciliary pools of adenylyl cyclases and channel proteins to enhance mechanosensitivity. TRPV4-calcium with a channel-biosensor fusion construct will be observed to distinguish ciliary from cytoplasmic influx. Finally, the inventors will determine whether mechanically induced ciliary cAMP synthesis occurs and distinguish the temporal relationship between ciliary and cytosolic cAMP using the ciliary-direct cAMP biosensor.

SA3: Elucidate the Role of Osteocyte Primary Cilia in the Propagation of Signals Through the Osteocyte Network and Downstream Regulation of Bone Turnover.

Working Hypothesis: Manipulating primary cilia sensitivity biases osteocytes towards osteogenic signaling while maintaining osteocyte function in activating bone cells and transmitting intercellular signals. Ciliary knockouts and pharmacologically enhanced primary cilia will be tested in terms of four aspects of bone physiology: (1) inter-osteocyte signaling with an ex vivo load-induced calcium signal propagation model; (2) osteocyte-osteoblast signaling in a co-culture model; (3) fatigue-induced osteoclastogenesis; and (4) disuse bone loss and recovery in a transient paralysis model. The expected outcomes are in vivo evidence of the potential to sensitize osteocyte primary cilia (SA1), identification of osteocyte interciliary signaling mechanisms to inform development of new therapeutic strategies (SA2) and determination of the impact of targeting osteocyte primary cilia on anabolic and catabolic signaling (SA3). This will have a positive impact to advance bone mechanobiology and contribute to the development of a more complete armamentarium to treat osteoporosis.

### Results

SA1: Therapeutic Manipulation of the Physical and Biochemical Composition of the Primary Cilium Microdomain and Quantification of the Resulting Change in Load-Induced Bone Formation In Vivo.

Introduction:

Previously the focus was to further the understanding of how primary cilia-mediated mechanotransduction allows osteocytes to sense and respond to mechanical cues. The inventors demonstrated that the primary cilium plays a critical role in osteocyte mechanobiology and that key structural and molecular aspects of this unique microdomain regulate its function in vitro. The objective of this aim is to determine if the effects of agents shown to alter primary cilia mechanosensitivity in vitro can be translated in vivo to enhance bone adaptation. It will be achieved by testing the working hypothesis that biochemical potentiation of the ciliary microdomain increases load-induced bone formation. The approach will be to target primary cilia structure and mechanosensing proteins via subcutaneous injections of pharmacologic agents in mice. Primary cilia stiffness will be modulated by tubastatin treatment, length will be targeted with fenoldopam, and the TRPV4 ion channel will be agonized with 4αPDD and GSK1019670A and antagonized with RN-1734. Mouse long bones will then be subjected to compressive loading to induce bone formation and the bone adaptation response will be assessed by dynamic histomorphometry. The expectation is that the inventors will show that treatment with primary cilia manipulating agents can alter the bone response to loading, indicating the primary cilium as not only a potential, but a viable and promising target for bone disease therapeutics. Furthermore, this will demonstrate an in vitro to in vivo scheme for the development of primary cilia targeted interventions for conditions such as osteoporosis.

Preliminary Studies:

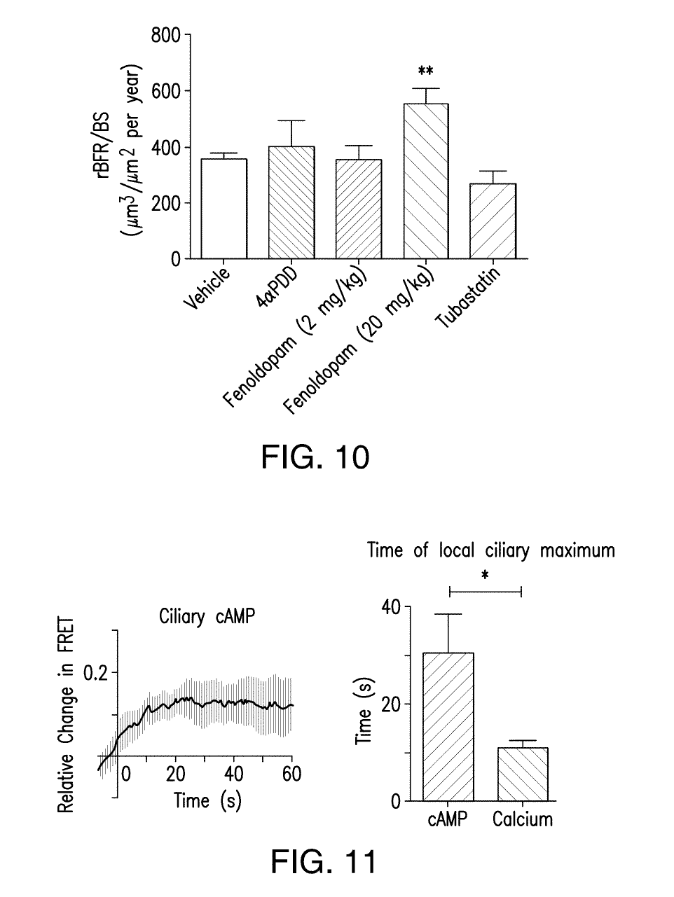

Skeletally mature C57BL/6J mice (16 weeks old) were administered subcutaneous injections of 4αPDD, fenoldopam, tubastatin A, or vehicle control and axial compressive ulnar loading followed by dynamic histomorphometry (FIG. 10). Results were quantified as the loaded limb relative to the non-loaded contralateral limb. The initial dose of 4αPDD, in only 3 animals, did not promote a significant increase in relative bone formation rate compared to vehicle control (rBFR/BS). A dose-response study was done for fenoldopam and there was a marked increase in response from 2 mg/kg to 20 mg/kg. Tubastatin treatment slightly decreased rBFR, and also resulted in an over 50% decrease (data not shown) in active mineralizing surface (rMS/BS). This effect was dramatic enough to warrant continued in vivo evaluation. Additionally, gross changes in size, weight, or temperament as a result of the injections was not observed. For fenoldopam (20 mg/kg) treated specimens, kidney histology showed no abnormalities or cysts and weight to body mass measurements were no different than vehicle controls. In addition, μCT analysis of these mice demonstrated no change in normal cortical bone properties including thickness, area, and moment of inertia (data not shown). This suggested that the treatments are well tolerated with no observed no unintended adverse consequences. These data support the working hypothesis that biochemically potentiating the ciliary microdomain increases load-induced bone formation, and that all of the required techniques to complete the aim are up-and-running in the inventors' hands.

SA2: Characterization of the Osteocyte Intraciliary Molecular Signaling Apparatus and Determination how its Dynamics are Affected by Altering Ciliary Structure and Composition.

Introduction:

While SA1 focused on immediate translation of pharmacological agents to enhance bone formation, SA2 focused on further elucidating the osteocyte cilia signaling microdomain to enable development of highly specific therapeutic strategies. Previously, the inventors identified a unique signaling mechanism of the primary cilium involving calcium influx through TRPV4 and cAMP production by AC6. Both, TRPV4 and AC6, have independently been shown to be important for mechanically-induced osteogenic signaling, though it is not known if they work in concert in the ciliary microdomain to regulate calcium/cAMP dynamics and downstream osteogenic signaling. A mechanistic understanding of osteocyte intraciliary signaling dynamics is impeding the refinement of molecular targets. Due to the amplifying nature of their signaling cascades, minute alterations in second messengers have large downstream impacts and strategies targeting them have promising pharmacological potency. Furthermore, there is great potential for therapeutic specificity in promoting bone growth since the osteocyte signaling apparatus is distinct from other cell types. The objective is to determine how osteocyte primary cilia microdomain potentiates signaling in response to mechanical stimulation. To achieve this objective the inventors will test the working hypothesis that calcium kinetics and cAMP signaling are coupled by adenylyl cyclases within the cilium and changes in their dynamics culminate in altered osteogenic gene expression. The approach is to utilize molecular techniques to characterize the interplay of intraciliary calcium/cAMP signaling. Specifically, novel biosensors will be utilized to quantify changes in calcium/cAMP signaling with alterations in axonemal stiffness, cilium length, and the presence and concentration of ciliary proteins believed to be involved in mechanotransduction. The hypothesis is that the composition and function of the osteocyte ciliary microdomain can be altered to enhance the calcium/cAMP signaling apparatus, thereby activating osteocytes independently of other cell types to encourage bone growth.

Preliminary Studies:

A mechanism was developed to detect ciliary cAMP levels specifically by fusing a FRET-based Epac1-cAMP biosensor to Arl13b, a protein that is naturally trafficked exclusively to the primary cilium. With shear flow, a ciliary cAMP increase was observed approximately 20 seconds following a spike in calcium (FIG. 11), suggesting the latter induced the former. The cytosolic cAMP levels decreased approximately 30 seconds following an increase in ciliary cAMP (data not shown). These results demonstrate that the ciliary and cytosolic signaling domains are distinct and it is feasible to study their dynamics using the novel biosensors.

To determine if AC6 couples calcium and cAMP in the osteocyte cilium, an overexpression vector was generated by inserting AC6 into a pcDNA3.2 backbone (pcDNA3.2+AC6). Two AC6 aspartic acid residues, Asp³⁸² and Asp⁴²⁶, were mutated via site directed mutagenesis to disrupt the calcium binding pocket (pcDNA3.2+AC6^{CalMut}). This mutation did not affect AC6's general catalytic activity by treating transfected cells with 10 μM of forskolin (which binds at an independent site) for 20 minutes and verified that cAMP production was not affected by the mutation (data not shown).

To identify AC isoforms other than AC6 that may mediate calcium/cAMP coupling, and ICC revealed that AC3 localizes to osteocyte but not kidney cilia (FIG. 14). This is the first examination of ciliary AC3 expression in non-excitable cells and is compelling evidence that there are unique components of the osteocyte cilium. To determine if calcium and cAMP are coupled and how calcium regulates inter-ciliary protein activity, plasmids of TRPV4, AC6 and AC3 were acquired and constructed. All plasmids were transferred into a common backbone, pcDNA3.2, to generate overexpression vectors. With site directed mutagenesis, the calmodulin binding domain of TRPV4 (Δ812-831) were deleted, and the calcium binding pocket of AC6 (Asp³⁸² and Asp⁴²⁶ to Ala) and AC3 (Asp³²⁴ and Asp³⁶⁸ to Ala) were mutated. TRPV4 mutations were previously validated. The results confirmed AC mutations did not affect protein activity by treating transfected cells with 10 μM of forskolin (which binds at an independent site) for 20 minutes and verified that cAMP production was not affected by the mutation (data not shown). Mutating TRPV4 and AC6 results in a decreased osteogenic response to flow compared to the wildtype overexpression vector, while mutated AC3 increased the osteogenic response to flow. These data demonstrate that calcium is involved in regulating osteogenic signaling through adenylyl cyclases and that all plasmids are in-hand and can be successfully transfected.

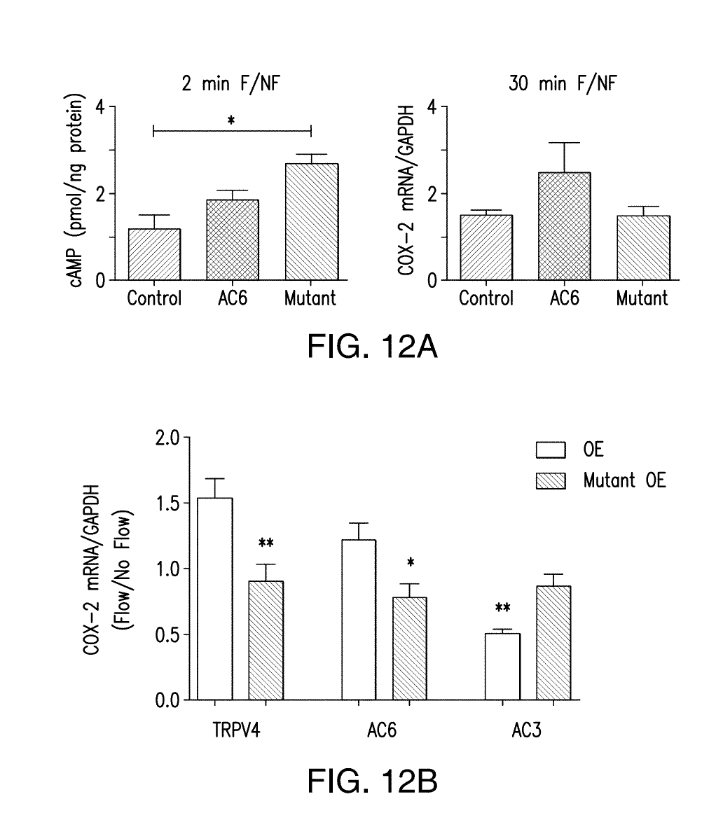

With flow, the mutant group had higher levels of cAMP (FIG. 12A), demonstrating that calcium inhibition was lost as expected and AC6 overexpression resulted in an enhanced osteogenic response that was lost in the mutants (FIG. 12A).

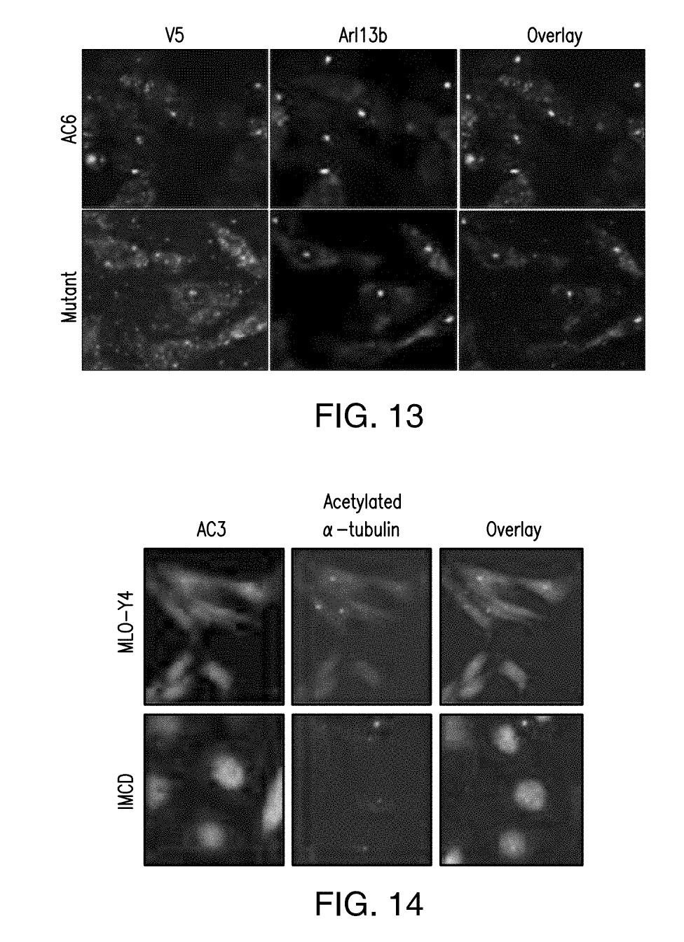

Next a mutant plasmid (pcDNA3.2+AC6^{VxPMut}) was generated to prevent trafficking of AC6 to the cilium. The inventors mutated two residues of a potential ciliary targeting sequence, a V×P motif, on the first intracellular loop. Indeed, AC6 overexpression in the cilium was lost with this mutation (FIG. 13). These results show that trafficking sequences can be genetically altered to selectively direct or prevent localization of proteins to influence osteogenesis.

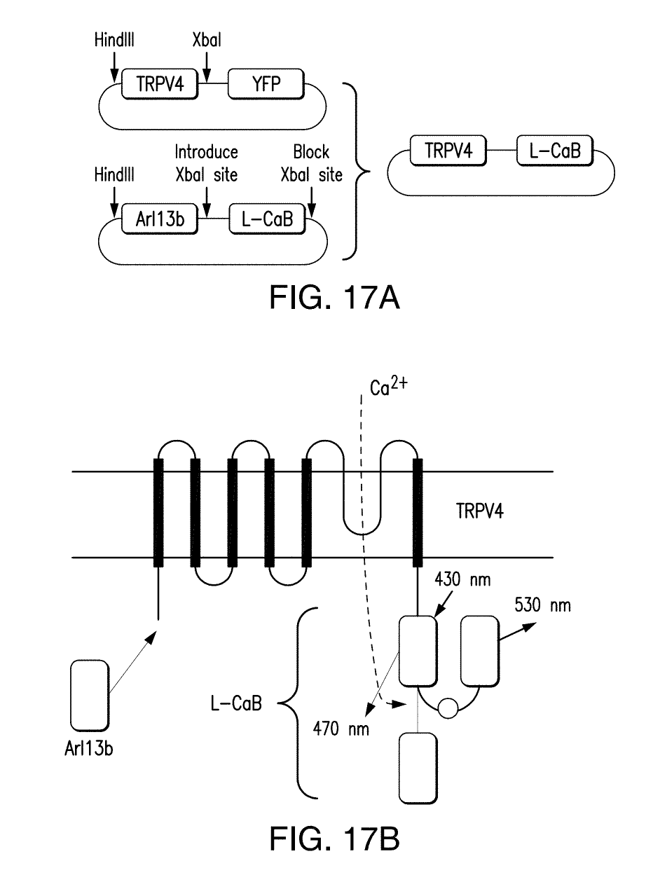

Finally, the calcium biosensor was linked to a TRPV4-YFP plasmid (gift from Heinrich Brinkmeier, University of Greifswald) to measure calcium entry independently from other calcium sources (FIG. 17). This same strategy will be employed to traffic TRPV4 exclusively to the cilium with Arl13b. To summarize the plasmid-based data, all sequences are in-hand and have been developed to varying degrees to validate the proposed experimental approach.

Collectively, the data suggest the osteocyte microdomain contains potentially unique proteins with specific functional domains that may become compelling targets for enhancing osteogenesis. They also suggest that a detailed understanding of ciliary calcium/cAMP dynamics will reveal new therapeutic candidates and that the molecular strategies to prosecute this aim are validated.

SA3: Elucidation of the Role of Osteocyte Primary Cilia in the Propagation of Signals Through the Osteocyte Network and Downstream Regulation of Bone Turnover.

Introduction:

With a long-term goal of therapeutically targeting the osteocyte primary cilium, it is critical to overcome the limitations of current osteoporosis treatments to only promote formation or inhibit resorption. For instance, bisphosphonates increase bone mass, but can lead increased microdamage accumulation eventually resulting in atypical femur fractures. While there is an emerging understanding of the role of osteocyte primary cilia in mechanosensing at the cellular and tissue level, it is crucial to future therapeutics to also understand osteocyte ciliary contribution to normal intercellular communication. The objective is to determine the role of osteocyte primary cilia in intercellular communication and if osteocyte mechanosensitivity can be manipulated while otherwise maintaining normal bone biology. This objective is pursued by testing the working hypothesis that the role of osteocytes in activating bone cells and transmitting intercellular signals is unchanged while manipulating their primary cilia. To that end, the approach examines three key aspects of bone biology: osteocyte intercellular communication, osteogenic signaling from osteocytes to osteoblasts and MSCs, and osteoclastogenesis. In Study 3.1 the impact on intracellular signaling in the model of the intact osteocyte network will be examined. Osteoclast activity due to disuse and overuse may occur through different mechanisms. For example overuse may produce microdamage, which could recruit osteoclasts independent of primary cilia. To address this, two independent models will be used: a newly developed repetitive loading model and the transient muscle-paralysis model. Additionally, as muscle function is regained in the paralysis model, normal bone mass is recovered, which allows us to examine load-induced bone formation with restoration of normal ambulation. Paracrine signaling between treated osteocytes and osteoblasts/MSCs will be examined to assess the effects on osteogenic signaling. The expectation is that altering osteocyte cilium mechanics will affect initial calcium signaling, promote osteogenic signaling, and allow normal osteoclast activation. These data would indicate that osteocyte ciliary mechanosensitivity can be targeted to shift the level of osteogenic signaling, while protecting normal bone rejuvenation.

Preliminary Data:

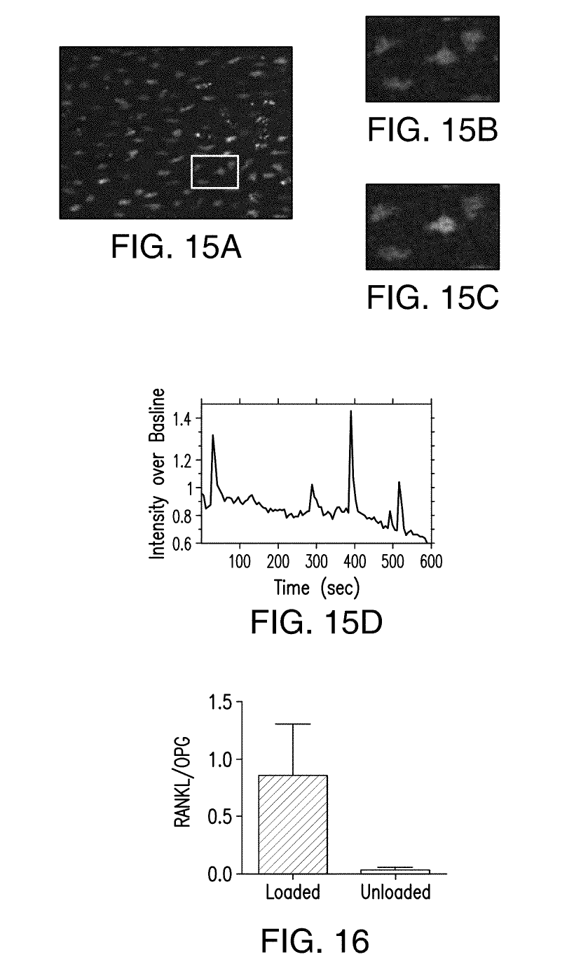

Whether manipulations of primary cilia have adverse consequences for osteocyte network response will be examined using a novel ex vivo model the inventors developed that tracks real-time intracellular calcium levels in an intact osteocyte network. An initial study was performed to validate the system and verify the osteocyte network response to physical loading (FIG. 15).

Osteoclastogenesis will be investigated using two potent stimuli: overloading and disuse. RANKL/OPG is a common readout for osteocytic regulation of osteoclastogenesis. For this study, a repetitive loading model of osteoclastogenesis was developed, crucial to enable exploitation of the extensive genetic mouse models. In a pilot experiment mice were subjected to a relatively small amount of cyclic overloading (roughly 1000 cycles). The mice responded to higher strains (3000 μtrain) and cycle number (˜1000) than the model of habitual loading used in SA1 with a dramatically increased RANKL/OPG mRNA ratios (FIG. 16), demonstrating that this model to study osteoclast activation, is up-and-running.

The impact of potentiating osteocyte primary cilia on osteocyte-osteoblast communication will be evaluated in a co-culture system. In a pilot study the inventors treated osteocytes with fenoldopam (lengthening agent) and tubastatin A (stiffening agent) to enhance or diminish mechanosensitivity, respectively. Manipulating osteocyte primary cilia affected paracrine signaling to osteoblasts and that it was achievable to both promote (fendoldopam) and diminish (tubastatin A) osteogenic response in osteoblasts. As shown in FIG. 8 (left panel) MLO-Y4 cells were seeded on a T75 flask. Fenoldopam (10 μM) was added 16 hours prior, while tubastatin A (5 μM) was added 4 hours before stimulation. Flasks with fresh media were placed in an incubator and rocked at 0.5 Hz with an amplitude of 1.5 cm for 12 hrs. Controls were placed in the same incubator, but not rocked. 12 mL of media from each flask was transferred to a 4-well plate of MC3T3-E1 osteoblasts. After 24 hours, increased osteogenic gene expression was observed with lengthening (fenoldopam) and decreased with stiffening (tubastatin) Flow over no-flow control data were used as comparison (n=4, ***p<0.001, Mean±SEM).

In summary the results suggest osteocyte cilia mechanosensing can be modulated to increase or decrease osteogenic activity. Furthermore the inventors have expanded the experimental capabilities in order to evaluate potential adverse effects of cilia targeted therapies on key aspects of bone biology.

### Example 4: High-Throughput Drug Screening Allows Identification of Ciliogenesis Modifying Compounds

In Example 1, two small molecules were identified, fenoldopam and lithium, as compounds that increase primary cilia length. Furthermore, it was found that the cells with longer cilia are more mechanosensitive. Primary cilia play a critical role in mechanosensing, chemosensing, and signal transduction in a myriad of cell types. Moreover, numerous different human conditions are being attributed to aberrant ciliogenesis. Disrupted ciliogenesis can result in improper embryogenesis, skeletal patterning disorders, polycystic kidney disease, anosmia, and retinitis pigmentosa, among numerous other conditions. While fenoldopam has been identified as a compound with potential to be repurposed for bone applications, the list of candidate therapeutics to treat such a range of ciliopathies is limited. In this Example high-throughput drug screening was utilized to recognize compounds that manipulate ciliogenesis. This work allows the identification of individual small molecules, as well as classes of compounds based on mechanism of action to help inform the development of novel cilia-targeted therapeutics.

As previously discussed herein, primary cilia are solitary immotile organelles serving chemo and mechanosensing roles in numerous different cell types. Primary cilia are comprised of microtubule doublets that are highly acetylated (Nguyen, A. M., Young, Y.-N. & Jacobs, C. R. Biol. Open 4, 1733-8 (2015)). Furthermore, primary cilia form a unique microdomain within the cell to which particular proteins localize, and through which many signaling pathways are transduced. Impaired primary cilia formation is being implicated with an increasing number of different health conditions being termed ciliopathies. The most notable of these being disrupted embryonic patterning and polycystic kidney disease (Davenport, J. R. & Yoder, B. K. Am J Physiol Ren. Physiol 289, F1159-69 (2005)). Primary cilia also serve chemosensing roles in olfaction as well as in neurons and in the retina controlling vision (McIntyre, J. C. et al. Nat. Med. 18, 1423-8 (2012)).

Due to the acetylated microtubule composition of the ciliary axoneme, it is anticipated that compounds targeting microtubule stability and microtubule acetylation, as well as cell cycle progression, will significantly impact ciliogenesis. Primary cilia formation is tightly regulated with the cell cycle, with ciliogenesis occurring during the G1 phase of the cell cycle (Avasthi, P. & Marshall, W. F. Differentiation 83, S30-S42 (2012)). As cells approach the G2 and S phase the cilium is disassembled before cell division. Taxol, and its derivatives, stabilize microtubules to prevent them from depolymerization, and can also arrest cells in the G1 phase, when ciliogenesis occurs (Arnal, I. & Wade, R. H. Curr. Biol. 5, 900-908 (1995)). Histone deacetylases (HDACs) increase α-tubulin acetylation to also stabilize microtubules (Portran, D., Schaedel, L., Xu, Z., Théry, M. & Nachury, M. V. Nat. Cell Biol. 19, 391-398 (2017)).

Primary cilia formation can be altered, pharmacologically, having a direct impact on cell function. Besschetnova et al. demonstrated that inhibiting intracellular calcium or stimulating the cAMP-PKA signaling pathway can significantly enhance primary cilia length (Besschetnova, T. Y. et al. Curr. Biol. 20, 182-7 (2010)). Recently it has been demonstrated that increasing primary cilia length with fenoldopam can potentiate mechanosensing in endothelial cells and osteocytes, resulting in enhanced nitric oxide production to reduce blood pressure, and increased osteogenic signaling, respectively (Hierck, B. P. et al. Dev Dyn 237, 725-735 (2008); Spasic, M. & Jacobs, C. R. Eur. Cell. Mater. 33, 158-168 (2017)). These works demonstrate that ciliogenesis can be pharmacologically enhanced, having a direct impact on cell function.

Targeting primary cilia is a potent way to manipulate cell activity, such as by sensitizing cells to chemical or mechanical stimuli. This has implications in combating diseases ranging from osteoporosis, atherosclerosis, and polycystic kidney disease, to cancer and primary ciliary dyskinesia. While several compounds are known to manipulate cilia length, the list is minimal. As described in this Example, high-throughput drug screening was utilized to expand the library of candidate bioactive small molecules that increase primary cilia length, to both further understanding of cilia biology and identify potential disease treating compounds.

In this Example high-throughput drug screening was performed to expand the list of candidate compounds that increase primary cilia length. A high-throughput screening system was established at the High-Throughput Screening Center at the Columbia Genome Center. This platform involves an automated process of cell culture on 384-well plates, drug treatment with 6931 compounds, staining, imaging, and image analysis.

The following materials and methods were used.

Cell Culture.

MLO-Y4 osteocytes were maintained on collagen I-coated dishes in MEMα (Life_Technologies) supplemented with 5% fetal bovine serum (FBS), 5% calf serum (CS), and 1% penicillin/streptomycin (P/S) at 37° C. and 5% CO2. Osteocytes were then seeded onto 384-well collagen-coated plates for 72 hours before fixation. Cells were treated, in triplicate, with 6931 biologically active small molecules from a collection of libraries for 16 hours before fixation. These libraries include Tocris, Sigma LOPAC, Selleck Chemical, Prestwick, and Spectrum commercially available compound libraries. All cell seeding and plate handling was performed with the Perkin Elmer cell:explorer workstation.

High-Throughput Imaging.

Cells were fixed in 4% paraformaldehyde and permeabilized with 0.1% Triton-X 100 (Sigma). 10% goat serum (vendor) was used for blocking. Cilia were stained using a monoclonal antibody against acetylated α-tubulin (1:20) from a C3B9 hybridoma cell line, and visualized with A F488 (1:1000, ThermoFisher). Nuclei were visualized with Hoechst. Cells were imaged using a GE INCell 2000 high-throughput imager at 40× magnification. 4 fields of view were taken per well.

Automated Cilia Detection and Measurement.

Each pair of nuclei and cilia images was first analyzed individually, then the entire population was examined to evaluate each compound's effect on cilia length and incidence. A custom MATLAB script was developed to perform all image analysis. This script first utilizes an enhanced implementation of a watershed transform, which is a segmentation tool which enables detection of similarly shaped overlapping particles in noisy images by the procedural whittling down of pixels found close to the edge of such particles. The augmentation of the algorithm incorporates thresholding for noise removal based on signal intensity and object size, nuclei dilation to exclude non-cellular artifacts, as well as skeletonization of cilia to quantify length. The results of the analysis are number of nuclei, number of cilia, and average cilia length in each image.

Identification and Classification of Hits.

Compounds were selected based on deviation from the population mean. Each well was normalized to vehicle control images on each plate, allowing the establishment of a normalized population mean. Hits were chosen based on compounds whose triplicate mean was furthest (based on standard deviation away) from the population mean. This was performed to identify the 105 individual compounds with the greatest deviation in terms of increased cilia length and can also be performed for changes in cilia incidence. Compounds were classified based on mechanism of action.

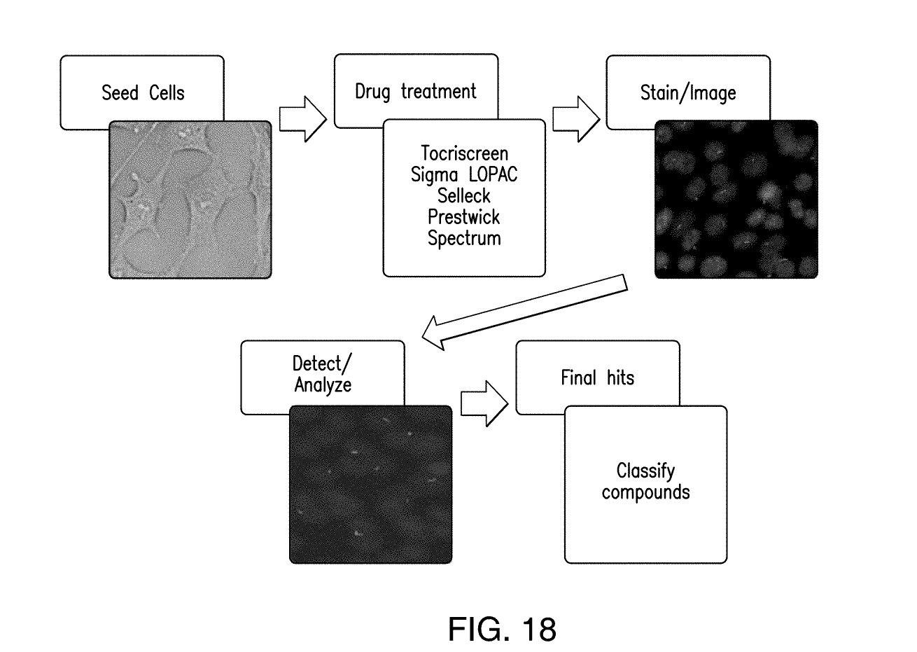

To identify compounds that modulate primary cilia formation a high-throughput platform was developed to automate cell culture, staining, imaging, and analysis, as shown in schematic form in FIG. 18. This platform was used to examine the effects of 6931 small molecule treatments on osteocyte primary cilia length and incidence. Osteocytes were chosen as the model cell type, as they form primary cilia of consistent length and incidence (Lee, K. L. et al. Cilia 4, 7 (2015); Spasic, M. & Jacobs, C. R. Eur. Cell. Mater. 33, 158-168 (2017); Malone, A. M. D. et al. PNAS 104 (33) 13325-13330 (2007)). Cells were cultured on 384-well plates, and treated with small molecules with known biologic activity, enabling classification of mechanisms of action. Drug treatment was applied for 16 hours at 10 μM concentration, performed in triplicate, before fixation and immunostaining of primary cilia. Cells were then imaged using the GE INCell 2000 High Content Analyzer, capturing 4 fields of view per well at 40× magnification. Additionally, each plate contains 8 wells of DMSO vehicle control.

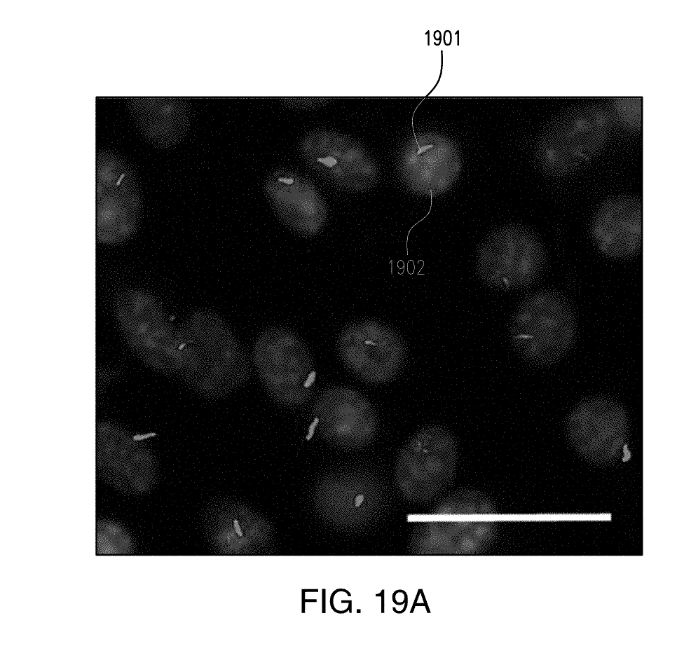

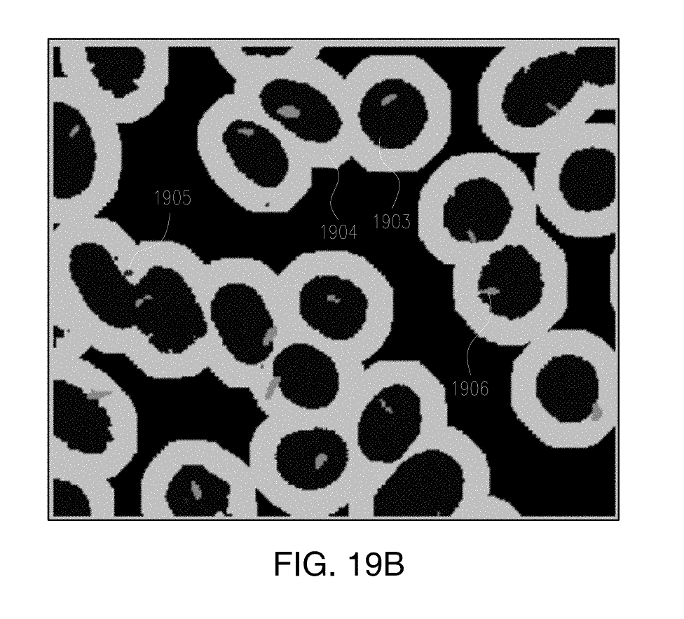

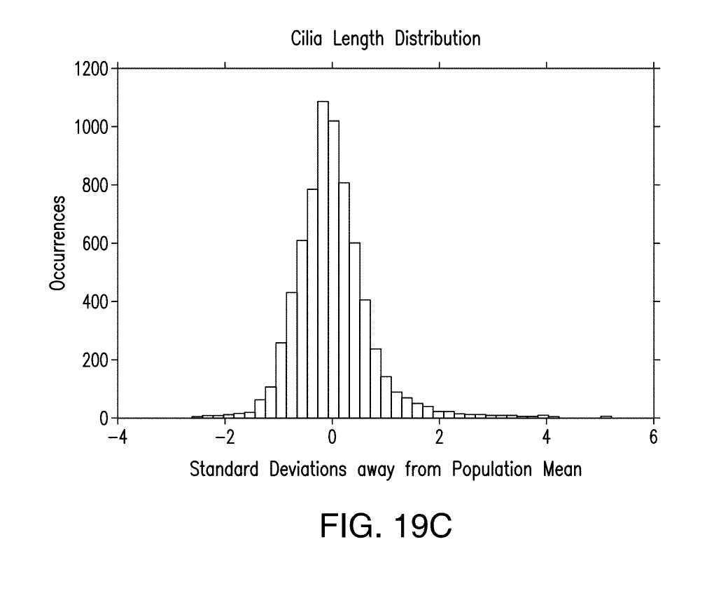

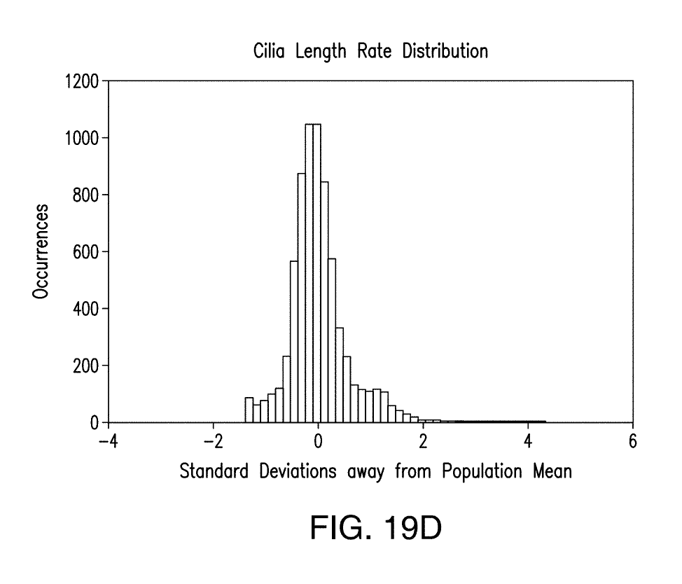

A MATLAB script was developed to detect and analyze changes in ciliogenesis. FIGS. 19A to 19D shows examples of primary cilia imaging, detection, and analysis. Cells stained for primary cilia (stained for acetylated α-tubulin; visualized with AF488, as example of which is indicated by 1901) and nuclei (stained with Hoescht, an example of which is indicated by 1902), and imaged at 40× magnification. A representative exemplary image (FIG. 19A) is cropped to better display features, scale bar represents 30 μm. A custom MATLAB script was used to identify nuclei and cilia for analysis (FIG. 19B). The script detects nuclei (an example of which is indicated by 1903) and applies a dilation mask to reject objects not associated with a cell (dilation mask indicated for example by 1904). Objects are further filtered based on size and signal intensity. Rejected objects are labeled (an example of which is indicated by 1905 and identified cilia are labeled (an example of which is indicated by 1906). Cilia length and incidence distributions are displayed as histograms of compound standard deviations from population mean (FIG. 19C and FIG. 19D). Nuclei and cilia images were binarized based on object size and signal intensity. A dilated mask was overlaid on the nuclei to identify only cilia that are associated with a nucleus, removing image artifacts. Cilia were skeletonized to quantify cilia length. Primary cilia incidence was quantified as the number of cilia versus nuclei per field of view.

Cilia length and incidence for each field of view were normalized to vehicle control on that individual plate. This allowed development of a normalized population average and standard deviation. Initially, hits were chosen as compounds with a normalized average 1.5 standard deviation above the population average (Kim, J. et al. Nature 464, 1048-1051 (2010)). To control for toxic effects only small molecules with at least 40% viability, based on number of nuclei versus control, were selected as hits. Furthermore, compounds repeated between multiple libraries eliciting the same effect on cilia length or incidence were excluded, such that only one of the repeated compounds was selected as a hit. For example, bufexamac was present in 3 libraries and was listed as a hit increasing length from each library but is only counted once for analysis of mechanism of action. This resulted in 103 individual compounds that increase primary cilia length, independent of changes in cilia incidence, and 92 compounds that increase cilia incidence independent of length. 18 individual compounds were conserved between the two lists.

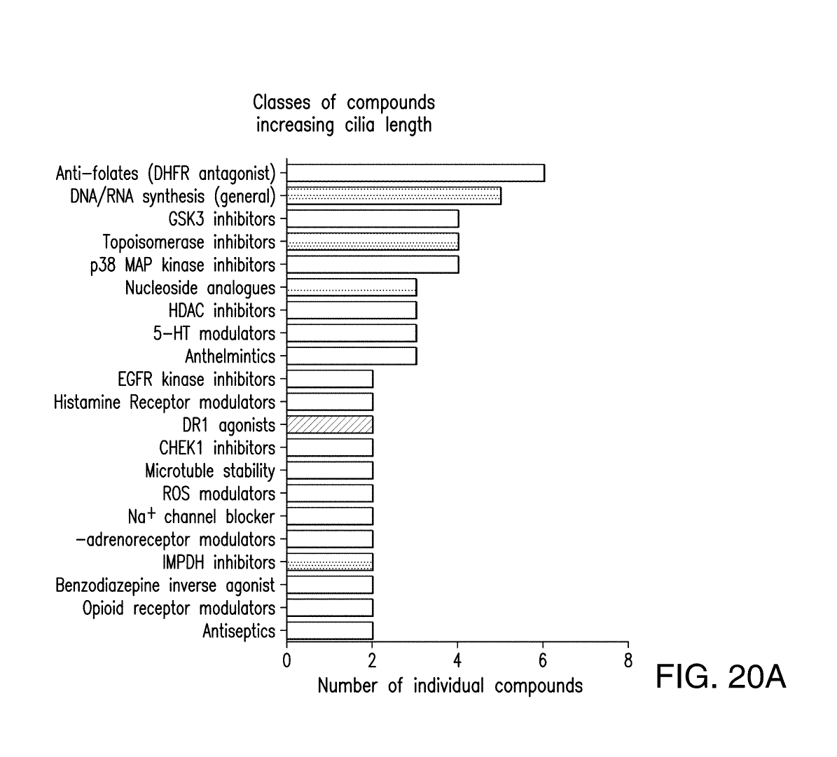

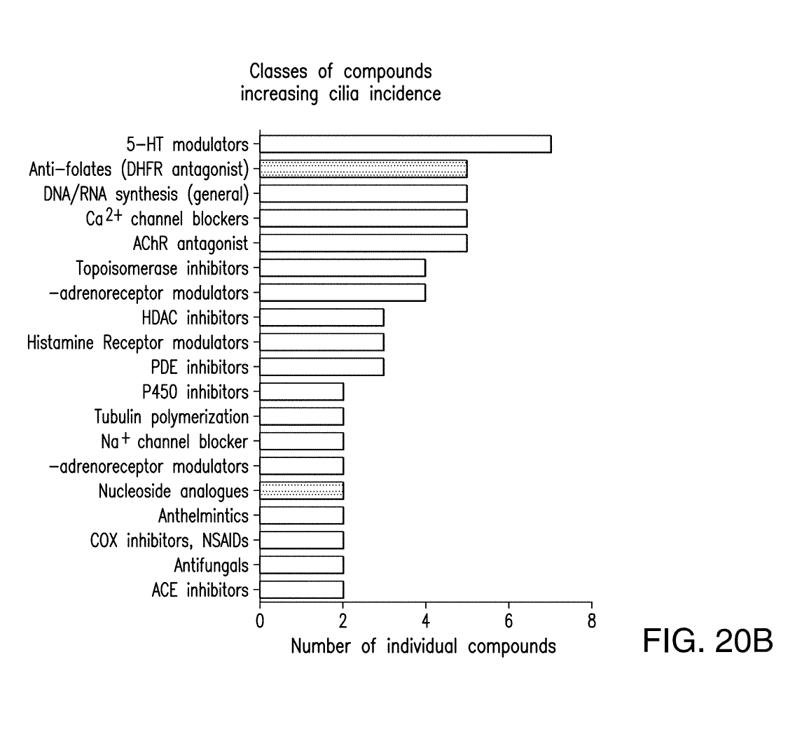

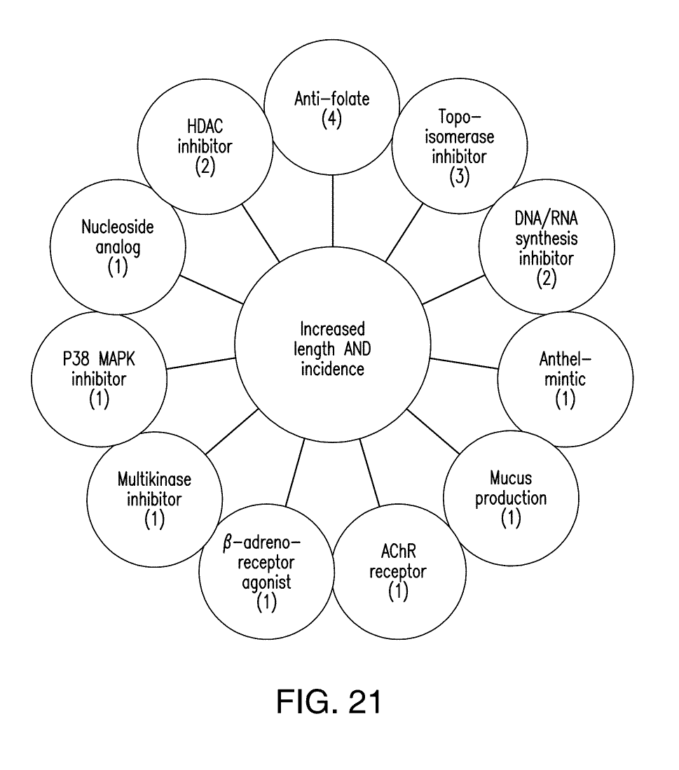

Compounds were classified based on mechanism of action to assess pathways through which primary ciliogenesis is altered. FIG. 20A is a graph reporting exemplary classification of compounds that increase cilia length. Compounds that increase cilia length were classified based on mechanism of action, independent of cilia incidence. Only classes with at least 2 hits are listed to demonstrate repeated mechanisms of action increasing cilia length. Classes marked with dots (anti-folates, general DNA/RNA synthesis inhibitors, topoisomerase inhibitors, and nucleoside analogues) all represent different compounds that inhibit DNA and RNA synthesis, slowing proliferation. Classes marked with slashes (DR1 agonists, Adenylyl cyclase modulators) represent compounds that increase AC activity and cAMP production. DR1 agonists have been demonstrated to stimulate production of ACs. FIG. 20B is a graph reporting exemplary classification of compounds that increase cilia incidence. Compounds that increase cilia incidence (independent of changes in cilia length) were classified based on mechanism of action, just as in FIG. 20A. The most common class of compounds was DNA/RNA synthesis inhibitors; including anti-folates, topoisomerase inhibitors, purine/pyrimidine (nucleoside) analogs, and general DNA/RNA synthesis inhibitors not falling into these other classes. It was also noticed that several distinct dopamine D1-like receptor (DR1) agonists increase length, as well as an adenylyl cyclase activator. As previously discussed, it has been demonstrated that DR1 agonists enhance adenylyl cyclase production. Additionally, compounds affecting microtubule stability, as well as GSK3 and HDAC inhibitors also increase primary cilia length. Of the 103 hits increasing length, 37 did not share a distinct pathway with another compound. 7 of these compounds have biologic activity, but their mechanisms of action have not been explicitly described in mammalian cells. These compounds comprise phytochemicals and flavonoids. 30 of 92 total incidence hits were also individual compounds. All hits increasing cilia length are listed in Table 2, along with the standard deviation away from the population average. All hits increasing cilia incidence are listed in Table 3, along with the standard deviation away from the population average. All hits increasing both cilia length and cilia incidence are listed in Table 4, along with the standard deviation away from the population average. These conserved hits are categorized by mechanism of action in FIG. 21.

TABLE 2
Compounds increasing cilia length.

| | | Average | |
|---|---|---|---|
| | | Length | |
| Drug Name | Vendor | SD Away | Mechanism of Action |
| (S)-(+)-Niguldipine | Tocris | 2.267094 | alpha1 antagonist, L-type |
| hydrochloride | | | Calcium channel blocker |
| | | | (cation channel) |
| 10- | Spectrum | 2.109686 | DNA Topoisomerase I |
| HYDROXYCAMPTOTHECIN | | | inhibitor |
| 110-Phenanthroline | Sigma_LOPAC | 1.837898 | metalloprotease inhibitor; |
| monohydrate | | | chelates iron, zinc, and |
| | | | other divalent metals |
| 2 4 - | Spectrum | 1.598346 | anthelmintic (anti- |
| DIHYDROXYCHALCONE 4 - | | | parasitic de-wormer) |
| GLUCOSIDE | | | |
| 2-Methoxyestradiol | SelleckChem_FDA | 1.619623 | depolymerizes |
| | | | microtubules, blocks |
| | | | HIF-1alpha |
| 5-BDBD | Tocris | 1.92035 | P2X4 receptor antagonist |
| | | | (purinergic receptor |
| | | | antagonist) |
| 5-fluorouracil | Prestwick | 1.775485 | Pyrimidine analog that |
| | | | stops DNA for forming in |
| | | | new and rapidly |
| | | | developing cells (acts as |
| | | | an inhibitor during the S- |
| | | | phase of cell division) |
| 62 -DIMETHOXYFLAVONE | Spectrum | 1.745088 | Other |
| | | | (phytochemical/flavanoid, |
| | | | undetermined) |
| 6-AMINONICOTINAMIDE | Spectrum | 2.008106 | NADP inhibitor (inhibitor |
| | | | of NADP-dependent |
| | | | enzyme, 6- |
| | | | phosphogluconate |
| | | | dehydrogenase); |
| | | | (interferes with |
| | | | glycolysis, resulting in |
| | | | ATP depletion) |
| AG 494 | Tocris | 1.57514 | EGFR-kinase inhibitor |
| | | | (epidermal growth factor |
| | | | receptor kinase inhibitor |
| AM 630 | Tocris | 1.74067 | CB2 selective inverse |
| | | | agonist (binds to the same |
| | | | receptor as an agonist, but |
| | | | does the opposite); (binds |
| | | | to peripheral cannabinoid |
| | | | 2 receptor) |
| Amethopterin (RS) | Prestwick | 2.927481 | formerly known as |
| | | | methotrexate; inhibits |
| | | | tetrahydrogolate |
| | | | dehydrogenase and |
| | | | prevents formation of |
| | | | tetrahydrofolate; involved |
| | | | in DNA regulation |
| AMINOPTERIN | Spectrum | 2.587185 | Dihydrofolate reductase |
| BAY 61-3606 hydrochloride | Sigma_LOPAC | 1.540018 | Spleen tyrosine kinase |
| hydrate | | | (Syk) inhibitor |
| Benzethonium chloride | Prestwick | 5.179286 | quaternary ammonium |
| | | | salt (surfactant, antiseptic, |
| | | | broad spectrum |
| | | | antimicrobial properties) |
| BETAHISTINE | Spectrum | 1.531649 | antagonist of histamine |
| HYDROCHLORIDE | | | H3 receptors (used for |
| | | | anti-vertigo treatment) |
| beta-TOXICAROL | Spectrum | 1.664142 | Other |
| | | | (phytochemical/flavanoid, |
| | | | undetermined) |
| BIO | Sigma_LOPAC | 2.019588 | GSK3 inhibitor; activates |
| | | | Wnt pathway |
| Bromhexine HCl | SelleckChem_FDA | 1.547211 | Increases production of |
| | | | mucus, makes phlegm |
| | | | thinner and less viscous; |
| | | | helps cilia transport |
| | | | phlegm out of the lungs |
| Bufexamac | Prestwick | 3.676165 | Anti-inflammatory, |
| | | | HDAC 6/10 inhibitor, |
| | | | COX inhibitor |
| BW 373U86 | Tocris | 1.553484 | potent, selective non- |
| | | | peptide delta-opioid |
| | | | receptor agonist |
| CP 339818 hydrochloride | Tocris | 1.714638 | non-peptide Kv1.3 |
| | | | channel antagonist |
| | | | (voltage-gated potassium |
| | | | channel); (cation channel) |
| CPT 11 | Tocris | 1.893689 | DNA Topoisomerase I |
| | | | inhibitor |
| CY 208-243 | Tocris | 2.56706 | Dopamine Receptor 1 |
| | | | agonist, selective over D2 |
| | | | receptors; stimulates ACs |
| CYCLOHEXIMIDE | Spectrum | 3.609172 | interferes with RNA |
| | | | translocation (movement |
| | | | of tRNA and mRNA) |
| | | | blocking translational |
| | | | elongation; used to inhibit |
| | | | protein synthesis |
| Cyclosporin A | Prestwick | 1.975398 | inhibits T-cell receptor |
| | | | signal transduction |
| | | | pathway; inhibits nitric |
| | | | oxide synthesis induced |
| | | | by interleukin 1alpha |
| DEACETOXY-7- | Spectrum | 1.843354 | Other |
| OXOGEDUNIN | | | (phytochemical/flavanoid, |
| | | | undetermined) |
| DEGUELIN(−) | Spectrum | 2.480167 | Rotenoid (derivative of |
| | | | Rotenone); inhibits |
| | | | NADH: ubiquinon |
| | | | oxidoreductase activity |
| DEHYDROCHOLIC ACID | Spectrum | 1.610536 | synthetic bile acid |
| | | | (carbonic anhydrase IV; |
| | | | G-protein coupled bile |
| | | | acid receptor 1; steroid 5- |
| | | | alpha-reductase 2) |
| DEOXYGEDUNIN | Spectrum | 2.160793 | agonist of TrkB |
| | | | (tropomysin receptor |
| | | | kinase B): (main receptor |
| | | | of brian-derived |
| | | | neurotrophic factor |
| | | | (BDNF)) |
| DEOXYKHIVORIN | Spectrum | 1.681439 | Other |
| | | | (phytochemical/flavanoid, |
| | | | undetermined) |
| DEOXYSAPPANONE B 73 - | Spectrum | 3.162781 | Other |
| DIMETHYL ETHER | | | (phytochemical/flavanoid, |
| ACETATE | | | undetermined) |
| DIHYDROCELASTRYL | Spectrum | 2.785454 | CHEK1 regulator |
| DIACETATE | | | (serine/threonine-specific |
| | | | protein kinase involved in |
| | | | DNA damage response) |
| Diphenyleneiodonium chloride | Tocris | 2.60257 | Activates adenylyl |
| | | | cyclases (through GPR3 |
| | | | but not GPR6 or 12); |
| | | | inhibits NO synthase; |
| Docetaxel | Prestwick | 3.416309 | microtubule stabilizer |
| | | | (synthetic analog of |
| | | | paclitaxel (taxol)) |
| Dorzolamide HCL | SelleckChem_FDA | 2.20499 | carbonic anhydrase |
| | | | inhibitor, used as an anti- |
| | | | glaucoma agent, and may |
| | | | have an effect on retinal |
| | | | cilia (rods) |
| Droxinostat | Sigma_LOPAC | 2.909825 | HDAC inhibitor (mainly |
| | | | HDAC6 and HDAC8, |
| | | | some affect on HDAC3, |
| | | | none on others) |
| DUARTIN DIMETHYL | Spectrum | 2.069704 | Other |
| ETHER | | | (phytochemical/flavanoid, |
| | | | undetermined) |
| ESTRADIOL CYPIONATE | Spectrum | 1.559704 | synthetic estrogen; |
| | | | estrogen receptor agonist |
| | | | (estradiol 17-beta- |
| | | | dehydrogenase 1) |
| Ethacridine lactate | SelleckChem_FDA | 2.114111 | antiseptic agent |
| monohydrate | | | (antibacterial, antibiotic) |
| Etonitazenyl isothiocyanate | Tocris | 1.857545 | affinity label specific for |
| | | | u opioid sites (micro |
| | | | opioid sites) |
| Floxuridine | SelleckChem_FDA | 1.672407 | Pyrimidine analog that |
| | | | stops DNA for forming in |
| | | | new and rapidly |
| | | | developing cells (acts as |
| | | | an inhibitor during the S- |
| | | | phase of cell division) |
| Ginkgolide B | Tocris | 1.581395 | PAFR antagonist |
| | | | (platelet-activating factor |
| | | | receptor) that inhibits |
| | | | platelet aggregation; |
| | | | blocks cell cycling |
| GW 1929 | Tocris | 1.650101 | activator of PPAR-gama |
| | | | (regulates fatty acid |
| | | | storage and glucose |
| | | | metabolism, marker of |
| | | | adipogenic |
| | | | differentiation) |
| HARMALOL | Spectrum | 1.628532 | beta-carboline; |
| HYDROCHLORIDE | | | benzodiazepine inverse |
| | | | agonist |
| HECOGENIN ACETATE | Spectrum | 2.209553 | inhibits production |
| | | | (reactive oxygen species); |
| HOMIDIUM BROMIDE | Spectrum | 1.694381 | same as Ethidium |
| | | | Bromide; trypanocidal |
| | | | agent; binds to nucleic |
| | | | acids; non-competitive |
| | | | inhibition of nicotinic |
| | | | acetylcholine receptors |
| HYCANTHONE | Spectrum | 2.895774 | intercalates into DNA and |
| | | | Inhibits RNA synthesis; |
| IC 261 | Sigma_LOPAC | 1.651397 | Casein kinase 1 (CK1) |
| | | | inhibitor |
| Indirubin-3′-oxime | Tocris | 2.474718 | Protein kinase inhibitor, |
| | | | GSK-3beta inhibitor |
| Irinotecan | SelleckChem_FDA | 1.657911 | Topoisomerase I inhibitor |
| ITRACONAZOLE | Spectrum | 2.154445 | antifungal, inhibits |
| | | | lanosterol 14alpha- |
| | | | demethylase to inhibit |
| | | | synthesis of ergosterol |
| Kenpaullone | Sigma_LOPAC | 3.279991 | CDK1/cyclin B and |
| | | | GSK3b inhibitor. |
| | | | Generates induced |
| | | | pluripotent stem cells |
| | | | when used in combo with |
| | | | reprogramming factors) |
| KHIVORIN | Spectrum | 1.722962 | Limonoid; partial agonist |
| | | | of adhesion G-protein |
| | | | coupled receptors |
| | | | (GPR56/ADGRG1 and |
| | | | GPR114/ADGRG5) |
| LEVULINIC ACID 3- | Spectrum | 2.3613 | Other |
| BENZYLIDENYL- | | | (phytochemical/flavanoid, |
| | | | undetermined) |
| Lonidamine | Tocris | 2.357239 | decreases glycolysis; |
| | | | antispermatogenic agent |
| Loratidine | Tocris | 2.260501 | Histamine H1 receptor |
| | | | agonist |
| Merbromin | Prestwick | 5.067169 | antiseptic agent; |
| | | | containing mercury |
| MERCAPTOPURINE | Spectrum | 1.721874 | derivative of purine |
| | | | (interferes with |
| | | | DNA/RNA synthesis); |
| | | | competes with |
| | | | hypoxanthine and |
| | | | guanine for the enzyme |
| | | | HGPRTase and is |
| | | | converted to thioinosinic |
| | | | acid (TIMP) |
| Methotrexate | Tocris | 3.09706 | chemotherapy agent, |
| | | | immune system |
| | | | suppressant; |
| | | | antimetabolite of the |
| | | | antifolate type; |
| | | | competitively inhibits |
| | | | DHFR (dihydrofolate |
| | | | reductase) |
| METHOXYAMINE | Spectrum | 1.910185 | binds to |
| HYDROCHLORIDE | | | apurinic/apyrimidinic |
| | | | DNA damage sites, |
| | | | inhibits base excision |
| | | | repair; increases DNA |
| | | | strand breaks |
| MONENSIN SODIUM | Spectrum | 1.721923 | Ionophorous antibiotic; |
| (monensin A is shown) | | | enables transport across |
| | | | lipid membranes |
| MONOBENZONE | Spectrum | 1.913581 | tyrosinase inhibitor; |
| | | | topical drug from medical |
| | | | depigmentation |
| Mycophenolate mofetil | SelleckChem_FDA | 1.579155 | inosine-5′- |
| (CellCept) | | | monophosphate |
| | | | dehydrogenase (IMPDH) |
| | | | inhibitor; inhibits |
| | | | DNA/RNA synthesis |
| Oxfendazole | SelleckChem_FDA | 1.573848 | Broad spectrum |
| | | | benzimidazole, an |
| | | | anthelmintic (anti- |
| | | | parasitic de-wormer) in |
| | | | veterinary use; sulfoxide |
| | | | metabolite of |
| | | | fenbendazole |
| PARAROSANILINE | Spectrum | 2.388812 | triarylmethane dye, one |
| PAMOATE | | | of the key components of |
| | | | fuchsin |
| PD 169316 | Sigma_LOPAC | 3.454305 | p38 MAP Kinase |
| | | | inhibitor |
| PD-407824 | Sigma_LOPAC | 1.510015 | Chk1 and Wee1 inhibitor |
| | | | (kinases) |
| Pemetrexed | SelleckChem_FDA | 2.765365 | Alimta (trade name); |
| | | | chemotherapy; similar to |
| | | | folic acid (DHFR |
| | | | inhibitor); inhibits |
| | | | enzymes used in purine |
| | | | and pyrimidine synthesis |
| | | | (thymidylate synthase |
| | | | [TS], dihydrofolate |
| | | | reductase [DHFR], |
| | | | glycinamide |
| | | | ribonucleotide |
| | | | formyltransferase |
| | | | [GARFT] |
| PERILLIC ACID (−) | Spectrum | 2.196372 | increases p21, Bax, and |
| | | | caspase-3; inducing |
| | | | apoptosis |
| PHA 767491 hydrochloride | Sigma_LOPAC | 1.724284 | Cdc7/CDK9 inhibitor |
| PIM 1 Inhibitor 2 | Tocris | 3.222586 | Pim-1 kinase inhibitor; |
| | | | Pim1: involved in cell |
| | | | cycle progression, |
| | | | apopotosis, and |
| | | | transcriptional activation |
| Piperlongumine | Sigma_LOPAC | 3.198224 | increases level of reactive |
| | | | oxygen species (ROS) |
| | | | and apoptosis in cancer |
| | | | cells but not normal. |
| | | | Possibly inhibits GSTP1 |
| | | | enzyme |
| Pralatrexate(Folotyn) | SelleckChem_FDA | 2.105394 | Chemotherapy; antifolate |
| PYRIMETHAMINE | Spectrum | 3.194135 | Dihydrofolate reductase |
| | | | inhibitor (DHFR) |
| PYRVINIUM PAMOATE | Spectrum | 2.834048 | Anthelmintic (anti- |
| | | | parasitic de-wormer) |
| QUINIDINE GLUCONATE | Spectrum | 1.82481 | Sodium channel alpha |
| | | | subunit blocker (cation |
| | | | channel blocker) |
| QUININE SULFATE | Spectrum | 1.801811 | Ferriprotoporphyrin IX |
| | | | inhibitor; reduction of |
| | | | oxygen intake, disruption |
| | | | of DNA replication and |
| | | | transcription via DNA |
| | | | intercalation |
| RESERPINE | Spectrum | 1.972238 | inhibitor of vesicular |
| | | | monoamine transporter |
| | | | (VMAT); affects |
| | | | norepinephrine, serotonin, |
| | | | and dompamine transport |
| Retinoic acid p-hydroxyanilide | Sigma_LOPAC | 2.487406 | Vitamin A acid analogue; |
| | | | antiproliferative, induces |
| | | | apoptosis |
| Ro 19-4605 | Tocris | 1.584464 | Benzodiazepine inverse |
| | | | agonist; binds to |
| | | | diazepam-sensitive (DS) |
| | | | and diazepame- |
| | | | insensitive (DI) GAPAa |
| | | | receptors |
| S 14506 hydrochloride | Tocris | 1.53968 | 5-HT1a agonist |
| S(−)-Atenolol | Sigma_LOPAC | 2.057248 | Beta1 adrenoreceptor |
| | | | antagonist |
| SANT-1 | Tocris | 1.811052 | Antagonist sonic |
| | | | hedgehog (SHH) and |
| | | | inhibits by binding to |
| | | | smoothened (Smo) |
| SB 203580 | Tocris | 1.796593 | p38 MAP Kinase |
| | | | inhibitor |
| SB 206553 hydrochloride | Tocris | 1.703544 | 5-HT2b/5-HT2c receptor |
| | | | antagonist |
| SB 228357 | Tocris | 2.332256 | 5-HT2b/5-HT2c receptor |
| | | | antagonist |
| SB 239063 | Tocris | 4.000291 | p38 MAP Kinase |
| | | | inhibitor |
| SB 408124 | Tocris | 2.398154 | non-peptide orexin OX1 |
| | | | receptor antagonist |
| SB 415286 | Tocris | 2.049715 | GSK-3 inhibitor |
| SECURININE | Spectrum | 1.502272 | GAGAa antagonist |
| SKF 77434 hydrobromide | Tocris | 1.527282 | Dopamine D1-like |
| | | | receptor agonist |
| SKF 86002 dihydrochloride | Tocris | 1.549216 | p38 MAP Kinase |
| | | | inhibitor |
| Sorafenib (Nexavar) | SelleckChem_FDA | 1.499834 | multikinase inhibitor of |
| | | | Raf-1, B-Raf, and |
| | | | VEGFR-2 |
| THIOGUANINE | Spectrum | 1.674629 | inosine-5′- |
| | | | monophosphate |
| | | | dehydrogenase (IMPDH) |
| | | | inhibitor; inhibits |
| | | | DNA/RNA synthesis |
| Topotecan hydrochloride | Sigma_LOPAC | 3.898638 | Topoisomerase I inhibitor |
| hydrate | | | |
| Triamterene | Sigma_LOPAC | 2.775097 | blocks epithelial sodium |
| | | | channels (ENaC) in a |
| | | | voltage dependent |
| | | | manner |
| Trifluridine (Viroptic) | SelleckChem_FDA | 1.772731 | DNA/RNA synthesis |
| | | | inhibitor |
| TULOBUTEROL | Spectrum | 2.059746 | beta2-adrenergic receptor |
| | | | agonist |
| Tyrphostin B44 (−) enantiomer | Tocris | 1.877017 | EGFR-kinase inhibitor |
| | | | (epidermal growth factor |
| | | | receptor) |
| Vinblastine | SelleckChem_FDA | 1.771483 | inhibits microtubule |
| | | | assembly |
| Vorinostat | Prestwick | 3.282859 | HDAC inhibitor (mainly |
| | | | HDAC1 and HDAC3, |
| | | | maybe some effect on |
| | | | other HDACs) |
| VU 0155069 | Tocris | 1.618479 | PLD1 inhibitor |
| | | | (phospholipase D1); |
| | | | inhibits cell migration |

TABLE 3
Compounds increasing cilia incidence.

| | | Average | |
|---|---|---|---|
| | | Incidence | |
| Drug Name | Vendor | SD Away | Mechanism of Action |
| Acadesine | SelleckChem_FDA | 1.846681 | AMPK activator (5′ AMP- |
| | | | activated protein kinase) |
| Acetanilide | SelleckChem_FDA | 1.696295 | inhibitor of hydrogen peroxide |
| (Antifebrin) | | | |
| Albendazole Oxide | SelleckChem_FDA | 2.01699 | tubulin polymerization inhibitor |
| (Ricobendazole) | | | |
| Amfebutamone | SelleckChem_FDA | 1.605458 | AChR; norepinephrine-dopamine |
| (Bupropion) | | | reuptake inhibitor |
| Aminophylline | Sigma_LOPAC | 2.589753 | PDE inhibitor (phosphodiesterase); |
| ethylenediamine | | | raises cAMP and activates PKA |
| Amiodarone HCl | SelleckChem_FDA | 3.380494 | Ion-channel blocker (broad |
| | | | spectrum) |
| Arcyriaflavin A | Tocris | 1.891508 | cdk4/cyclin D1 inhibitor |
| Aspartame | SelleckChem_FDA | 1.731531 | non-saccharide artificial sweetener |
| Atropine | SelleckChem_FDA | 1.694156 | AChR antagonist (muscarinic |
| | | | acetylcholine receptor) |
| Avobenzone (Parsol | SelleckChem_FDA | 1.501535 | absorbs UVA rays (used in |
| 1789) | | | sunscreen) |
| Azasetron HCl | SelleckChem_FDA | 1.652885 | 5-HT3 antagonist |
| Benidipine | SelleckChem_FDA | 1.811129 | calcium channel blocker |
| hydrochloride | | | (dihydropiridine) |
| Betaxolol | SelleckChem_FDA | 1.630679 | beta-1 adrenergic receptor blocker |
| hydrochloride | | | |
| (Betoptic) | | | |
| Bethanechol chloride | SelleckChem_FDA | 1.763703 | AChR agonist (muscarinic |
| | | | receptor) |
| Bifonazole | SelleckChem_FDA | 1.66407 | imidazole antifungal agent |
| Bromhexine HCl | SelleckChem_FDA | 1.88232 | Increases production of mucus, |
| | | | makes phlegm thinner and less |
| | | | viscous; helps cilia transport |
| | | | phlegm out of the lungs |
| Bufexamac | Prestwick | 2.326419 | Anti-inflammatory, HDAC 6/10 |
| | | | inhibitor, COX inhibitor |
| Bupivacaine | SelleckChem_FDA | 1.826848 | Sodium channel blocker (binds to |
| hydrochloride | | | voltage-gated Na channels, blocks |
| (Marcain) | | | sodium influx) |
| Cimetidine | SelleckChem_FDA | 1.561816 | Histamine receptor H2 antagonist |
| (Tagamet) | | | |
| Cladribine | SelleckChem_FDA | 1.603977 | DNA/RNA synthesis (adenosine |
| | | | deaminase inhibitor) |
| Cleviprex | SelleckChem_FDA | 1.837172 | calcium channel blocker |
| (Clevidipine) | | | (dihydropiridine) |
| CPT 11 | Tocris | 1.633435 | Topoisomerase I inhibitor |
| Detomidine HCl | SelleckChem_FDA | 1.92214 | alpha2-adrenergic agonist |
| Dexmedetomidine | SelleckChem_FDA | 2.924328 | alpha2-adrenergic agonist |
| HCl (Precedex) | | | (adrenergic receptors) |
| Dextrose (D-glucose) | SelleckChem_FDA | 1.517602 | simple sugar (other) |
| Diclazuril | SelleckChem_FDA | 1.667569 | antifection; anticoccidial drug |
| Diphenhydramine | SelleckChem_FDA | 1.928039 | Histamine H1 receptor antagonist |
| HCl (Benadryl) | | | |
| Dipyridamole | SelleckChem_FDA | 2.055435 | PDE inhibitor (phosphodiesterase) |
| (Persantine) | | | |
| Droxinostat | Sigma_LOPAC | 2.259524 | HDAC inhibitor (mainly HDAC6 |
| | | | and HDAC8, some affect on |
| | | | HDAC3, none on others) |
| Edaravone (MCI- | SelleckChem_FDA | 1.536994 | antioxidant (other) |
| 186) | | | |
| Fenofibrate (Tricor | SelleckChem_FDA | 2.949682 | PPAR-alpha activator (peroxisome |
| Trilipix) | | | proliferator-activated receptor |
| | | | alpha) |
| Fenoprofen calcium | SelleckChem_FDA | 3.367532 | COX-2 inhibitor; NSAID |
| Floxuridine | SelleckChem_FDA | 1.579591 | Pyrimidine analog that stops DNA |
| | | | for forming in new and rapidly |
| | | | developing cells (acts as an |
| | | | inhibitor during the S-phase of cell |
| | | | division) |
| Flutamide (Eulexin) | SelleckChem_FDA | 2.034848 | Androgen receptor antagonist |
| Gabexate mesylate | SelleckChem_FDA | 1.870428 | Serine protease inhibitor |
| Geniposide | SelleckChem_FDA | 4.33545 | iridoid glycoside (other) |
| Genistein | SelleckChem_FDA | 1.523506 | PTK inhibitor (protein tyrosine |
| | | | kinase) |
| Hydroxyurea | SelleckChem_FDA | 1.987889 | DNA/RNA synthesis inhibitor |
| (Cytodrox) | | | |
| Idoxuridin | SelleckChem_FDA | 1.798224 | Nucleoside analogue; DNA/RNA |
| | | | synthesis inhibitor |
| Iloperidone (Fanapt) | SelleckChem_FDA | 1.774571 | 5-HT2 Receptor (serotonin)/ |
| | | | dopamine D2 receptor antagonist |
| Irinotecan | SelleckChem_FDA | 1.680718 | Topoisomerase I inhibitor |
| Irsogladine | SelleckChem_FDA | 1.543361 | PDE inhibitor (phosphodiesterase) |
| Isoprenaline | SelleckChem_FDA | 1.622508 | beta-adrenergic receptor agonist |
| hydrochloride | | | |
| Ivabradine HCl | SelleckChem_FDA | 1.610631 | Calcium channel blocker |
| (Procoralan) | | | |
| Lafutidine | SelleckChem_FDA | 1.618716 | Histamine H2 receptor antagonist |
| Levosimendan | SelleckChem_FDA | 1.520002 | Calcium sensitizer |
| Licofelone | SelleckChem_FDA | 1.935552 | COX inhibitor; NSAID |
| Lomustine (CeeNU) | SelleckChem_FDA | 2.227746 | DNA/RNA synthesis inhibitor |
| LY 364947 | Tocris | 1.590856 | TGF-beta receptor 1 inhibitor |
| Medroxyprogesterone | SelleckChem_FDA | 2.659248 | proestrogen receptor agonist |
| acetate | | | |
| Methotrexate | Tocris | 2.423671 | chemotherapy agent, immune |
| | | | system suppressant; antimetabolite |
| | | | of the antifolate type; |
| | | | competitively inhibits DHFR |
| | | | (dihydrofolate reductase) |
| Metoprolol tartrate | SelleckChem_FDA | 1.601192 | Beta-1 adrenergic receptor |
| | | | antagonist |
| Metronidazole | SelleckChem_FDA | 2.09186 | DNA/RNA synthesis inhibitor |
| (Flagyl) | | | |
| Mirtazapine | SelleckChem_FDA | 1.681189 | 5-HT Receptor antagonist |
| (Remeron Avanza) | | | |
| Mycophenolate | SelleckChem_FDA | 1.654129 | inosine-5′-monophosphate |
| mofetil (CellCept) | | | dehydrogenase (IMPDH) inhibitor; |
| | | | inhibits DNA/RNA synthesis |
| Nefiracetam | SelleckChem_FDA | 1.552041 | GABAergic system enhancer |
| (Translon) | | | (other) |
| Nicorandil (Ikorel) | SelleckChem_FDA | 1.556012 | Potassium channel activator |
| Nitrendipine | SelleckChem_FDA | 1.542139 | Calcium channel (dihydropyridine) |
| NXY 059 | Tocris | 1.838152 | Free-radical trapping agent |
| Olanzapine (Zyprexa) | SelleckChem_FDA | 1.609438 | 5-HT2 Receptor (serotonin)/ |
| | | | dopamine D2 receptor antagonist |
| Ondansetron (Zofran) | SelleckChem_FDA | 1.506065 | 5-HT3 receptor antagonist |
| Oxfendazole | SelleckChem_FDA | 2.267721 | anthelmintic (anti-parasitic de- |
| | | | wormer) |
| Oxibendazole | SelleckChem_FDA | 4.084284 | Broad spectrum benzimidazole, an |
| | | | anthelmintic (anti-parasitic de- |
| | | | wormer) in veterinary use; |
| | | | sulfoxide metabolite of |
| | | | fenbendazole |
| Ozagrel HCl | SelleckChem_FDA | 1.683075 | Thromboxane A(2) (TXA(2)) |
| | | | snthetase inhibitor; P450 (e.g. |
| | | | CYP17) |
| Pefloxacin mesylate | SelleckChem_FDA | 1.681721 | Topoisomerase (IV) inhibitor |
| Pemetrexed | SelleckChem_FDA | 2.082984 | Alimta (trade name); |
| | | | chemotherapy; similar to folic acid |
| | | | (DHFR inhibitor); inhibits |
| | | | enzymes used in purine and |
| | | | pyrimidine synthesis (thymidylate |
| | | | synthase [TS], dihydrofolate |
| | | | reductase [DHFR], glycinamide |
| | | | ribonucleotide formyltransferase |
| | | | [GARFT] |
| Phenindione | SelleckChem_FDA | 1.687887 | Vitamin K antagonist |
| (Rectadione) | | | |
| Pioglitazone | SelleckChem_FDA | 2.52042 | PPARgamma agonist; P450 (e.g. |
| hydrochloride | | | CYP17) |
| (Actos) | | | |
| Pralatrexate(Folotyn) | SelleckChem_FDA | 2.192505 | Chemotherapy; antifolate (DHFR |
| | | | inhibitor) |
| Proparacaine HCl | SelleckChem_FDA | 1.522795 | Sodium channel antagonist |
| | | | (voltage-gated Na channels) |
| Propylthiouracil | SelleckChem_FDA | 1.658653 | Thyroperoxidase and 5′-deiodinase |
| | | | inhibitor |
| PYRIMETHAMINE | Spectrum | 1.761932 | Dihydrofolate reductase inhibitor |
| | | | (DHFR) |
| Ranolazine (Ranexa) | SelleckChem_FDA | 1.564839 | pFOX inhibitor (partial fatty acid |
| | | | oxidation); calcium uptake |
| | | | inhibitor via sodium/calcium |
| | | | channel |
| Rivastigmine tartrate | SelleckChem_FDA | 1.663812 | AChR inhibitor |
| (Exelon) | | | (acetylcholinesterase) |
| Rosuvastatin calcium | SelleckChem_FDA | 1.643692 | HMG-CoA Reductase inhibitor |
| (Crestor) | | | |
| S(−)-Atenolol | Sigma_LOPAC | 2.17694 | Beta1 adrenoreceptor antagonist |
| SB 239063 | Tocris | 1.794902 | p38 MAP Kinase inhibitor |
| Sodium butyrate | SelleckChem_FDA | 2.3981 | HDAC inhibitor |
| Sorafenib (Nexavar) | SelleckChem_FDA | 1.677517 | Raf-1, B-Raf, and VEGFR-2 |
| | | | multikinase inhibitor |
| Sulfamethoxazole | SelleckChem_FDA | 1.766832 | DHFR synthesis |
| TAME | SelleckChem_FDA | 2.32474 | APC inhibitor (anaphyse- |
| | | | promoting complex) |
| Tebipenem pivoxil | SelleckChem_FDA | 1.857371 | Antifection (broad spectrum |
| (L-084) | | | antibiotic) |
| Temocapril HCl | SelleckChem_FDA | 1.876515 | ACE inhibitor |
| Tiotropium Bromide | SelleckChem_FDA | 1.593179 | AChR (muscarinic) antagonist |
| hydrate | | | |
| Tolnaftate | SelleckChem_FDA | 1.5099 | Antifungal (Antifection) |
| Topotecan | Sigma_LOPAC | 2.750407 | Topoisomerase I inhibitor |
| hydrochloride | | | |
| hydrate | | | |
| Tranexamic acid | SelleckChem_FDA | 1.58212 | Lysine analog |
| (Transamin) | | | |
| Trifluridine | SelleckChem_FDA | 2.117341 | DNA/RNA synthesis inhibitor |
| (Viroptic) | | | |
| Tropisetron | SelleckChem_FDA | 1.694477 | 5-HT3 receptor antagonist |
| Valsartan (Diovan) | SelleckChem_FDA | 1.647861 | ACE inhibitor; angiotensin II |
| | | | blocker |
| Vinblastine | SelleckChem_FDA | 3.124003 | Inhibits microtubule assembly; |
| | | | suppresses nAChR activity |
| Zolmitriptan (Zomig) | SelleckChem_FDA | 1.520897 | 5-HT(1B/1D) receptor agonist |

TABLE 4
Compounds increasing both cilia length and cilia incidence.

| | | Average | Average | |
|---|---|---|---|---|
| | | Length | Incidence | |
| Drug Name | Vendor | SD Away | SD Away | Mechanism of Action |
| Bromhexine HCl | SelleckChem_FDA | 1.547211 | 1.88232 | Increases production of |
| | | | | mucus, makes phlegm |
| | | | | thinner and less viscous; |
| | | | | helps cilia transport |
| | | | | phlegm out of the lungs |
| Bufexamac | Prestwick | 3.676165 | 2.326419 | Anti-inflammatory, |
| | | | | HDAC 6/10 inhibitor, |
| | | | | COX inhibitor |
| CPT 11 | Tocris | 1.893689 | 1.633435 | Topoisomerase I |
| | | | | inhibitor |
| Droxinostat | Sigma_LOPAC | 2.909825 | 2.259524 | HDAC inhibitor (mainly |
| | | | | HDAC6 and HDAC8, |
| | | | | some affect on HDAC3, |
| | | | | none on others) |
| Floxuridine | SelleckChem_FDA | 1.672407 | 1.579591 | Pyrimidine analog that |
| | | | | stops DNA for forming |
| | | | | in new and rapidly |
| | | | | developing cells (acts as |
| | | | | an inhibitor during the |
| | | | | S-phase of cell division) |
| Irinotecan | SelleckChem_FDA | 1.657911 | 1.680718 | Topoisomerase I |
| | | | | inhibitor |
| Methotrexate | Tocris | 3.09706 | 2.423671 | chemotherapy agent, |
| | | | | immune system |
| | | | | suppressant; |
| | | | | antimetabolite of the |
| | | | | antifolate type; |
| | | | | competitively inhibits |
| | | | | DHFR (dihydrofolate |
| | | | | reductase) |
| Mycophenolate | SelleckChem_FDA | 1.579155 | 1.654129 | inosine-5′- |
| mofetil (CellCept) | | | | monophosphate |
| | | | | dehydrogenase |
| | | | | (IMPDH) inhibitor; |
| | | | | inhibits DNA/RNA |
| | | | | synthesis |
| Oxfendazole | SelleckChem_FDA | 1.573848 | 2.267721 | anthelmintic (anti- |
| | | | | parasitic de-wormer) |
| Pemetrexed | SelleckChem_FDA | 2.765365 | 2.082984 | Alimta (trade name); |
| | | | | chemotherapy; similar to |
| | | | | folic acid (DHFR |
| | | | | inhibitor); inhibits |
| | | | | enzymes used in purine |
| | | | | and pyrimidine synthesis |
| | | | | (thymidylate synthase |
| | | | | [TS], dihydrofolate |
| | | | | reductase [DHFR], |
| | | | | glycinamide |
| | | | | ribonucleotide |
| | | | | formyltransferase |
| | | | | [GARFT] |
| Pralatrexate(Folotyn) | SelleckChem_FDA | 2.105394 | 2.192505 | Chemotherapy; |
| | | | | antifolate (DHFR |
| | | | | inhibitor) |
| PYRIMETHAMINE | Spectrum | 3.194135 | 1.761932 | Dihydrofolate reductase |
| | | | | inhibitor (DHFR) |
| S(−)-Atenolol | Sigma_LOPAC | 2.057248 | 2.17694 | Beta1 adrenoreceptor |
| | | | | antagonist |
| SB 239063 | Tocris | 4.000291 | 1.794902 | p38 MAP Kinase |
| | | | | inhibitor |
| Sorafenib (Nexavar) | SelleckChem_FDA | 1.499834 | 1.677517 | Raf-1, B-Raf, and |
| | | | | VEGFR-2 multikinase |
| | | | | inhibitor |
| Topotecan | Sigma_LOPAC | 3.898638 | 2.750407 | Topoisomerase I |
| hydrochloride | | | | inhibitor |
| hydrate | | | | |
| Trifluridine | SelleckChem_FDA | 1.772731 | 2.117341 | DNA/RNA synthesis |
| (Viroptic) | | | | inhibitor |
| Vinblastine | SelleckChem_FDA | 1.771483 | 3.124003 | Inhibits microtubule |
| | | | | assembly; suppresses |
| | | | | nAChR activity |

Using the high-throughput drug screening platform, compounds and classes of compounds that enhance ciliogenesis were identified. This has potential to elucidate molecular pathways and targets for therapeutic development of compounds to treat conditions marked by impaired ciliogenesis. Impaired cilia formation has consequences in cell signaling and homeostasis, as well as chemo and mechanosensing.

The final list of hits is comprised only of individual compounds, yet many compounds were repeated in different libraries with the same mechanism of action. This screen utilizes multiple different commercially available libraries, so there is overlap in compounds between libraries. Because of this, many compounds identified by the 1.5 standard deviation threshold are repeated, with 17 of the 103 compounds increasing cilia length having at least one repetition in another library. For example, both bufexamac and methotrexate are listed as hits from Selleck and Prestwick libraries. 92 compounds were identified to increase cilia incidence (independent of cilia length), with 8 of these containing library repetitions with similar effects on incidence. Compound repetition between libraries demonstrates robustness of the entire drug treatment, imaging, and analysis platform.

The results from the screen focus on compounds that increase primary cilia length and incidence. The screening data were also analyzed for compounds that decrease cilia length and incidence. This, however, elicited almost no hits. Compounds that decreased cilia length or incidence by at least 1.5 standard deviations also caused cell death. Of compounds that decreased cilia incidence, none retained at least 40% cell viability. Many ciliopathies are characterized by impaired cilia formation and function. Because of this, the analysis of the drug screen results was focused on compounds that increase cilia length and incidence.

The high-throughput screening platform does not necessarily exclude compounds not listed as hits. Through this platform compounds were only tested at 10 μM concentrations over a 16 hour timeframe. Lithium was not classified as a hit, however lithium increases cilia length over 16 hours, at 500 μM concentration. Cilia length changes have also been reported with 3 hours of drug treatment with calcium channel blockers (gadolinium, 30 μM) or adenylyl cyclase activators (forskolin, 100 μM) (Besschetnova, T. Y. et al. Curr. Biol. 20, 182-7 (2010)). While a full dose-response on this complete bioactive library of 6931 compounds was not conducted, classifying compounds based on mechanism of action still allows examination of pathways through which ciliogenesis may be modified. The largest group of hits that increase cilia length are DNA and RNA synthesis inhibitors, including anti-folates, topoisomerase inhibitors, purine/pyrimidine analogues, and other less specific DNA/RNA inhibitors. As previously introduced, ciliogenesis coincides with the cell cycle, and deciliation occurs with the initiation of DNA synthesis (Tucker et al. Cell 17, 527-535 (1979)). Additionally, topoisomerase inhibitors have also previously been demonstrated to enhance ciliogenesis (Khan et al. Oncotarget 7, 9975-92 (2016)). Purine and pyrimidine analogues additionally prevent the continuation of the cell cycle, allowing enhanced ciliogenesis (Sigmond, J. & Peters, G. J. Nucleosides. Nucleotides Nucleic Acids 24, 1997-2022 (2005)). The role of anti-folates and dihydrofolate reductase inhibitors (DHFRs) on primary cilia formation has not been explicitly examined, but because DHFR inhibitors impair DNA/RNA synthesis, this result is not unexpected (Johnston et al. Mol. Cell. Biol. 6, 3373-81 (1986)). In fact, certain doses of prenatal exposure to the DHFR inhibitor methotrexate can result in methotrexate embryopathy, characterized by altered patterning and embryogenesis, consistent with altered primary cilia function (Chapa et al. Obstet. Gynecol. 101, 1104-7 (2003)).

Drug classification also supports a purported dopamine receptor-adenylyl cyclase-calcium-related pathway to enhance cilia length. Several distinct compounds that act as agonists of dopamine D₁-like receptors (DR1), as well as compounds that stimulate adenylyl cyclase activity, are demonstrated as hits in the screen. A described herein, fenoldopam, a DR1 agonist enhances cilia length (Kathem, S. H. et al. J. Geriatr. Cardiol. 11, 63-73 (2014); Spasic, M. & Jacobs, C. R. Eur. Cell. Mater. 33, 158-168 (2017)). Furthermore, it has been demonstrated that DR1 agonists enhance AC6 production, which results in increased cAMP production (Yu, P. et al. Cell. Signal. 26, 2521-2529 (2014)). Besschetnova et al. reported that blocking calcium channels or stimulating cAMP production and PKA activation all increase primary cilia length, in a process that is impaired with AC6 disruption (Besschetnova, T. Y. et al. Curr. Biol. 20, 182-7 (2010)). AC6 is a calcium inhibited isoform of adenylyl cyclase that localizes to the primary cilium (Kwon, R. Y., et al. FASEB J. 24, 2859-68 (2010)). Through the screen general calcium channel blockers that increase cilia length were also identified. Together with existing literature, the compounds classified through the screen help to demonstrate a pathway through which DR1 agonists stimulate an increase in AC6 production to promote cilia elongation. AC6 plays a critical role in load-induced bone formation and mechanotransduction (Kwon, R. Y., et al. FASEB J. 24, 2859-68 (2010); Lee, K. L. et al. FASEB J. 28, 1157-1165 (2014)). The results demonstrate that DR1 and AC agonists have potential use in promoting bone formation for the treatment of bone diseases such as osteoporosis.

Histone deacetylases have been implicated in cilia formation, and suggest a length-stiffness functional relationship. It has been previously demonstrated that HDACs activity, including HDAC2 and HDAC6 activity, results in cilia disassembly (Kobayashi, T. et al. EMBO Rep. 18, 334-343 (2017); Ran, J. et al. Sci. Rep. 5, 1-13 (2015)). Primary cilia contain heavily acetylated microtubules, and this acetylation stabilizes microtubules from disassembly (Portran, D. et al. Nat. Cell Biol. 19, 391-398 (2017)). HDAC6 inhibition also stabilizes cilia to prevent mechanically-induced disassembly (Thompson, C. L. et al. Osteoarthr. Cartil. 22, 490-498 (2014)). Cilia length enhances mechanosensitivity; however, increased microtubule acetylation by HDAC6 inhibition increases primary cilia stiffness to abrogate primary cilia-mediated mechanotransduction (Nguyen, A. M. et al. Biol. Open 4, 1733-8 (2015); Spasic, M. & Jacobs, C. R. Eur. Cell. Mater. 33, 158-168 (2017)). This distinction suggests that a relationship exists whereby both cilia length and stiffness can independently impact its function as a cellular mechanosensor.

The drug screening platform utilized osteocytes as a model system, but modulating primary ciliogenesis has broad implications in treating numerous human conditions. Increasing cilia length can enhance cell mechanosensitivity. In the context of bone, increasing cilia length augments osteogenic activity and may have potential as a bone disease therapeutic strategy (Spasic, M. & Jacobs, C. R. Eur. Cell. Mater. 33, 158-168 (2017)). Increasing cilia length also has implications in treating hypertension resulting from disrupted cilia mechanosensing (Kathem, S. H. et al. J. Geriatr. Cardiol. 11, 63-73 (2014)). Modulating ciliogenesis can also restore chemosensing. A mouse model of diminished IFT88 production leads to impaired cilia formation, resulting in disrupted olfaction (McIntyre, J. C. et al. Nat. Med. 18, 1423-8 (2012)). When ciliogenesis is recovered—using an adenovirus to promote IFT88 expression—olfaction in the mice is restored. Primary ciliogenesis has also been linked to tumor progression, with many cancer types reported to lose primary cilia (Hassounah, N. B. et al. Clin. Cancer Res. 18, 2429-35 (2012)). In particular, premalignant and invasive breast cancer cells both display significantly fewer cilia than normal breast tissue (Menzl, I. et al. Cilia 3, 7 (2014)). Primary cilia regulate hedgehog signaling, which is known to be disrupted in tumorigenesis (Hassounah, N. B. et al. Clin. Cancer Res. 18, 2429-35 (2012)). Thus, restoring ciliogenesis also has significant potential in slowing cancer progression. Understanding ciliogenesis and identifying modulators of this process are critical to developing therapeutic strategies for a myriad of different human health conditions.

### Example 5: Osteocyte Cilia Mediate Paracrine Signaling Between Cells within Bone

Additional details related to Example 2 are discussed in this Example.

Bone maintenance and mechanotransduction is regulated by intercellular communication between the cells within bone. Osteocytes residing deep within bone tissue have been demonstrated to be highly mechanosensitive, and it is believed that these cells coordinate intercellular signaling to direct bone formation and osteogenic differentiation. As described herein, primary cilia are critical osteocyte mechanosensors. In this Example osteocytes were mechanically stimulated and the conditioned media was used to culture bone-forming osteoblasts and mesenchymal stem cells (MSCs), to study mechanically-induced intercellular paracrine signaling. This Example demonstrates that the osteocyte cilium, and ciliary mechanosensing proteins, play a critical role in initiating the mechanotransduction events that result in pro-osteogenic paracrine signaling to osteoblasts. Furthermore, this Example demonstrates that enhancing osteocyte primary cilia-mediated mechanotransduction with fenoldopam augments osteoblast activity and MSC osteogenic differentiation. Together, this demonstrates a regulation of intercellular bone signaling through manipulation of the osteocyte cilium.

The function of osteocytes was long unknown, since osteoblasts actively form new bone material and mesenchymal stem cells can differentiate into osteoblasts, while osteocytes resorb bone. Osteocytes reside deep within the lacuno-canalicular network in bone, comprising over 90% of all bone cells (Schaffler, M. B. & Kennedy, 0. D. Curr. Osteoporos. Rep. 10, 118-25 (2012)). Studies now suggest that this dominant cell type actually serves a mechanosensing role in bone; and moreover, that osteocytes direct signaling to other cells within bone. Notably, Tatsumi et al. utilized an in vivo osteocyte-specific ablation model and found impaired bone mechanotransduction in mice lacking osteocytes (Tatsumi, S. et al. Cell Metab 5, 464-475 (2007)).

The prevailing paradigm is that osteocytes sense mechanical forces, and transduce these signals to direct the activity of the other cell types within bone. It is believed that osteocytes signal to osteoblasts, osteoclasts, and MSCs through soluble paracrine signals as well as direct cell communication. However, only recently have these phenomena been explicitly demonstrated (Vezeridis, P. S. et al. Biochem. Biophys. Res. Commun. 348, 1082-1088 (2006); Hoey, D. A. et al. Biochem. Biophys. Res. Commun. 412, 182-187 (2011)). Osteocytes subjected to pulsatile fluid flow were found to secrete soluble factors into the media that increase osteoblast proliferation, potentially through activation of a nitric oxide pathway (Vezeridis, P. S. et al. Biochem. Biophys. Res. Commun. 348, 1082-1088 (2006)). More recently, it was found that osteocytes exposed to oscillatory fluid flow display enhanced signaling to MSCs, increasing MSC osteogenic differentiation, migration, and proliferation (Hoey, D. A. et al. Biochem. Biophys. Res. Commun. 412, 182-187 (2011); Brady, R. T. et al. Biochem. Biophys. Res. Commun. 459, 118-123 (2015)). Moreover, also it has recently been demonstrated that paracrine factors secreted by mechanically stimulated osteocytes can elicit epigenetic changes in MSCs that promote osteogenic differentiation (Chen, J. C. et al. J. Biomech. Eng. 137, 20902 (2015)).

While it was previously described that primary cilia play a significant role in osteocyte mechanosensing, it is unclear how this initial mechanotransduction event may be transduced into downstream signaling to other cell types. As mentioned herein, impairing primary cilia formation disrupts osteocyte mechanosensing, and whole bone mechanotransduction.

Similarly, knockdown and knockout of ciliary proteins such as stretch-activated ion channels (TRPV4, PC2) and mechanotransductive proteins (AC6) abrogates osteocyte and whole bone mechanosensing. Furthermore, it was demonstrated that the osteocyte cilium can be pharmacologically sensitized to mechanical stimulation. It is unclear, however, how these proteins or manipulation of the osteocyte cilium impact the intercellular bone signaling axis.

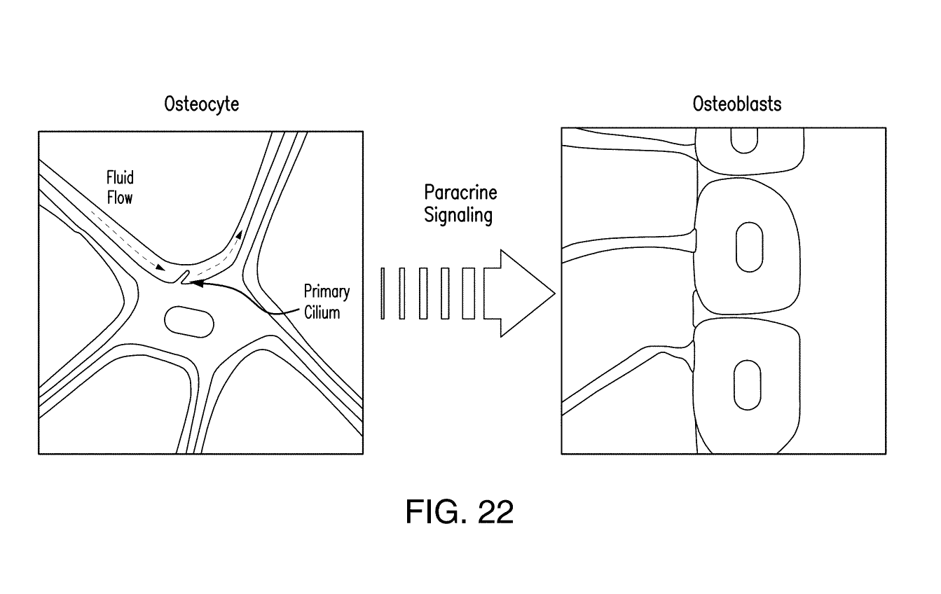

FIG. 22 is a schematic illustrating exemplary flow-induced osteogenic paracrine signaling. A prevailing paradigm in bone mechanotransduction is that osteocytes sense mechanical stimuli and transduce these signals to direct osteoblast-mediated bone formation and mesenchymal stem cell osteogenic differentiation. In this Example soluble paracrine signaling between mechanically stimulated osteocytes and osteoblasts/MSCs was examined.

To study intercellular paracrine signaling, conditioned media were collect from mechanically stimulated osteocytes and use to culture osteoblasts and MSCs. This model allows RNA and pharmacologic manipulation of cilia and ciliary proteins specifically in osteocytes, while quantifying resultant osteogenic changes in osteoblasts and MSCs. To study soluble paracrine signaling from mechanically stimulated cells a rocking platform was utilized to supply oscillatory fluid flow and consistently collect conditioned media. Based on the size of the culture vessel, amount of media, placement on rocker, and rocking angle and frequency, the peak shear stress can accurately be calculated (Zhou, X. et al. J Biomech 43, 1598-1602). Using this scheme, not only can the initial ciliary mechanosensing events be studied, but also how the resultant downstream signaling impacts intercellular communication. The following materials and methods were used.

Cell Culture.

MLO-Y4 osteocytes were cultured on collagen I-coated dishes in MEMα (Life Technologies) supplemented with 5% FBS, 5% CS, and 1% P/S at 37° C. and 5% CO₂. Osteocytes were seeded at 2800 cells/cm₂ for 72 hours before application of fluid flow. MC3T3 osteoblasts were cultured in MEMα supplemented with 10% FBS and 1% P/S. Osteoblasts were seeded at 2000 cells/cm₂ 72 hours before receiving conditioned media. C3H10T1/2 MSCs were cultured in DMEM low glucose (Life Technologies) supplemented with 10% FBS and 1% P/S, and seeded at 1100 cells/cm₂ 72 hours before culture with conditioned media 150. Osteocytes were treated with 10 μM fenoldopam mesylate (Sigma) for 16 hours or 5 μM tubastatin (Sigma) for 3 hours, prior to experimentation.

Mechanical Stimulation.

Osteocytes were cultured on rectangular flasks (8.2×9.2 cm; 12.5 ml of media) on a rocking platform which oscillated at a frequency of 0.33 Hz with an amplitude of 1.3 cm, supplying 0.08 Pa shear stress for 2, 6, 12, or 24 hours, FIG. 4.2. Shear stress generated by oscillatory fluid flow was calculated as previously performed ₁₅₃. 12 hours of fluid flow was used in all experiments. Cells were also cultured in static conditions as a no flow control. Conditioned media was collected from osteocytes immediately upon completion of rocking, and used to culture osteoblasts or MSCs for 24 or 48 hours, respectively.

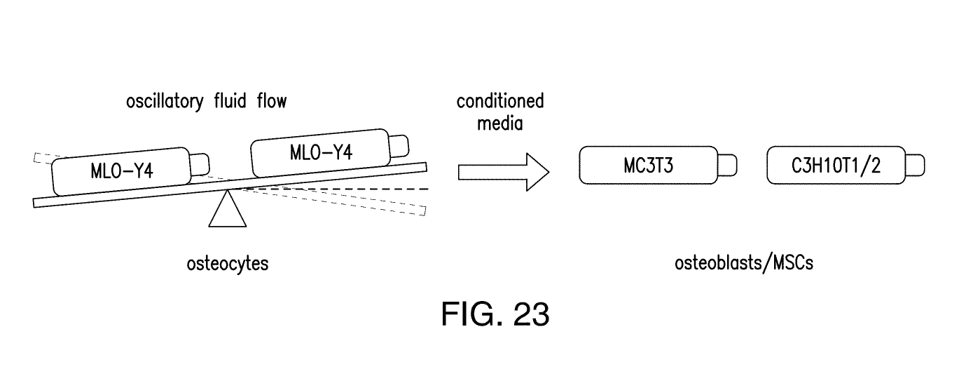

FIG. 23 is a schematic illustrating an exemplary model of flow-induced paracrine signaling. To study mechanically-induced osteogenic paracrine signaling between osteocytes and osteoblasts/MSCs, a rocker platform was utilized to perform conditioned media studies. In this platform, MLO-Y4 osteocytes are placed on a rocker to apply oscillatory fluid flow mechanical stimulation. The conditioned media is then collected and used to culture MC3T3 osteoblasts and C3H10T1/2 mesenchymal stem cells. With this set-up osteocyte primary cilia-mediated mechanotransduction can be manipulated (such as by siRNA-mediated knockdown or pharmacologic challenge) and the resulting changes in paracrine signaling leading to altered osteoblast and MSC activity can be studied. All results are compared relative to static (no flow) osteocytes.

RNA Interference in Osteocytes.

Gene silencing was performed only in osteocytes, 48 hours prior to application of fluid flow, by siRNA-mediated knockdown. All data was compared to scramble siRNA control. Primary cilia were disrupted by IFT88 siRNA (5′-CCAGAAACAGATGAGGACGACCTTT-3′) (SEQ ID NO: 1) (Spasic, M. & Jacobs, C. R. Eur. Cell. Mater. 33, 158-168 (2017)). Adcy6 (5′-CCTGCCACCTACAACAGCTCAATTA-3′) (SEQ ID NO: 2), Pkd2 (5′-CCTCTTGGCAGTTTCAGCCTGTAAA-3′) (SEQ ID NO: 3), and TRPV4 (5′-GATGGACTGCTCTCCTTCTTGTTGA-3′) (SEQ ID NO: 4) were disrupted as previously performed (Lee, K. L. et al. Cilia 4, 7 (2015); Spasic, M. & Jacobs, C. R. Eur. Cell. Mater. 33, 158-168 (2017)).

mRNA Expression.

Osteoblasts and MSCs were washed with PBS and total mRNA was isolated using TriReagent (Sigma), immediately following 24 hours (48 hours for MSCs) of culture in conditioned media from mechanically stimulated (or static control) osteocytes. Total mRNA was converted to cDNA by TaqMan reverse transcriptase (Applied Biosystems). Gene expression was analyzed by quantitative real-time PCR using primers and probes (Life Technologies) for analysis of osteopontin, OPN (Mm00436767_m1), osterix, OSX (Mm04209856_m1), and GAPDH (4351309). Samples and standards were run in triplicate, and all gene expression was normalized to GAPDH endogenous control.

Analysis.

All data were analyzed with one-way ANOVA followed by Bonferroni post-hoc correction. Values are reported as mean±SEM, with p<0.05 considered statistically significant. Sample size, n, represents biological replicates.

Results:

Increased Duration of Flow Enhances Signaling to Osteoblasts.

Cultured osteocytes were placed on the rocker platform and subjected to oscillatory fluid flow for 2, 6, 12, or 24 hours, each timepoint with a corresponding static control. Media was collected and used to culture osteoblasts for 24 hours. At each timepoint osteoblasts demonstrated a significant increase in osteopontin (OPN) mRNA expression compared to static controls (FIG. 6). 12 hours of rocking was used for all further experiments.

Intercellular Signaling is Impaired with Cilia/Associated Protein Disruption.

The role of osteocyte primary cilia-mediated mechanotransduction on osteogenic intercellular paracrine signaling was examined. Primary cilia formation was disrupted using siRNA-mediated knockdown of IFT88, while mechanotransduction proteins were impaired by knockdown of the ciliary stretch-activated ion channels TRPV4 and PC2, or knockdown of AC6. Knockdown of osteocyte cilia or ciliary mechanosensing proteins significantly impairs mechanically-induced pro-osteogenic paracrine signaling to osteoblasts (FIG. 8).

Fenoldopam Treatment Enhances Intercellular Signaling.

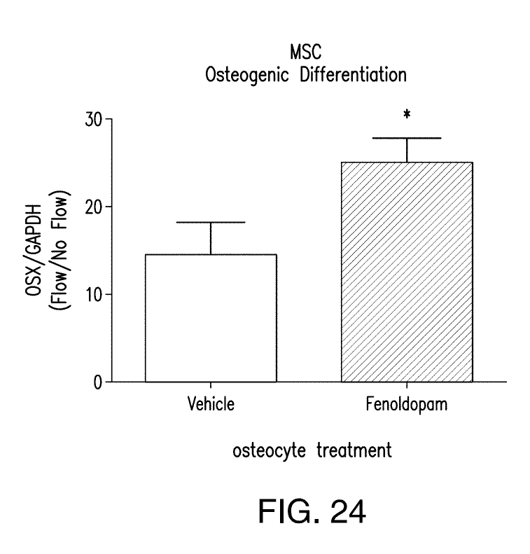

It was examined how potentiating osteocyte mechanosensitivity may direct intercellular osteogenic signaling. Osteocytes were treated with fenoldopam to increase cilia length and enhance mechanosensitivity, and subjected to oscillatory fluid flow. The collected media was used to culture osteoblasts or MSCs for 24 or 48 hours, respectively. Media collected from the fenoldopam treated osteocytes significantly enhanced osteoblast osteogenic activity, as measured by osteopontin gene expression (FIG. 8), and significantly enhanced MSC osteogenic differentiation, as measured by osterix (OSX) expression (FIG. 24). FIG. 24 is a graph reporting exemplary results of MSC osteogenic differentiation by pharmacologically manipulating osteogenic intercellular communication. Osteocytes were treated with fenoldopam or vehicle control, and the resulting response of osteoblasts and MSCs was examined. FIG. 24 shows fenoldopam treatment sensitizes osteocyte mechanosensitivity to enhance mechanically stimulated pro-osteogenic paracrine signaling promoting osteoblast activity (A), and MSC osteogenic differentiation. Additionally, HDAC6 (histone deacetylase 6) inhibition increases ciliary bending stiffness to decrease mechanosensitivity (Nguyen, A. M. et al. Biol. Open 4, 1733-8 (2015)). Osteoblasts cultured with conditioned media from mechanically stimulated osteocytes treated with the HDAC6 inhibitor, tubastatin, displayed impaired osteopontin expression (FIG. 8).

This Example demonstrates that cilia, and cilia-associated proteins not only impact osteocyte mechanosensitivity, but the entire mechanotransduction cascade leading to pro-osteogenic intercellular communication. By manipulating osteocyte mechanosensitivity, the intercellular signaling axis between cells within bone can be directed. Intercellular communication within bone utilizes several different paracrine factors to mediate osteogenic signaling. Mechanically stimulated osteocytes have been demonstrated to increase production of prostaglandin E2 and cAMP, as well as release of nitric oxide, Ca²⁺, and ATP (Schaffler, M. B. et al. Calcif. Tissue Int. 94, 5-24 (2014); Robling, A. G. et al. J Biol Chem 283, 5866-5875 (2008)). Expression of larger peptides involved in bone formation such as IGF-1, RANKL, and OPG have also been demonstrated to be increased in osteocytes exposed to physical stimulation (Malone, A. M. D. et al. Proc. Natl. Acad. Sci. 104, 13325-30 (2007); Lean, J. M., et al. Am. J. Physiol. 268, E318-27 (1995)). One limitation of the model used in this Example is that MLO-Y4 osteocytes do not produce sclerostin, a significant regulator of osteoblast activity (Nguyen, A. M. & Jacobs, C. R. Bone 54, 196-204 (2013); Robling, A. G. et al. J Biol Chem 283, 5866-5875 (2008)). As previously described, sclerostin is produced by osteocytes as a negative regulator of osteoblast activity. However, this Example demonstrates that sclerostin production by osteocytes is not exclusively necessary for the regulation of osteoblast activity by paracrine signaling.

Paracrine signaling is not the sole method of intercellular communication. Osteocyte dendritic processes extend throughout the lacuno-canalicular network, allowing for direct cell contact between osteocytes, and other cells within bone. This allows for the formation of gap junctions between cells through which ions such as Na₊, K₊, Ca₂₊, and Mg₂₊ can be transported to neighboring cells (Schaffler, M. B. et al. Calcif. Tissue Int. 94, 5-24 (2014)). Connexins make up hemichannels which form gap junctions mediating intercellular signaling. In both osteocytes and osteoblasts connexin 43 (Cx43) has been demonstrated to open in response to fluid flow, enabling signal transduction between neighboring cells (Plotkin, L. I. et al. Curr. Osteoporos. Rep. 13, 67-72 (2015)). While the model does not recapitulate the entire complexity of bone, the data do demonstrate that manipulation of cellular mechanosensitivity can have profound impacts on pro-osteogenic intercellular signaling.

By manipulating osteocyte primary cilia and ciliary associated proteins, intercellular communication can be directly mechanically-induced. Knockdown of AC6 impairs osteocyte mechanosensing, in a similar manner to impaired ciliogenesis by IFT88 knockdown (Kwon, R. Y. et al. FASEB J. 24, 2859-68 (2010); Spasic, M. & Jacobs, C. R. Eur. Cell. Mater. 33, 158-168 (2017)). It has also been shown that polycystin 2 is involved in NO production in osteocytes exposed to oscillatory fluid flow (Xu, H. et al. J. Biomech. 47, 387-391 (2014)). In vivo, skeletal deletion of the polycystin ion channel complex impairs whole bone mechanosensitivity (Xiao, Z. et al. FASEB J. 25, 2418-2432 (2011)). Also, knockdown of TRPV4 impairs osteocyte mechanosensing (Lee, K. L. et al. Cilia 4, 7 (2015)). TRPV4 has been implicated with a key role in cartilage mechanosensing, and mutations in this channel have been associated with numerous skeletal dysplasias (O'Conor, C. J. et al. Proc. Natl. Acad. Sci. U.S.A 111, 1316-21 (2014); Nishimura, G. et al. Am. J. Med. Genet. Part C Semin. Med. Genet. 160 C, 190-204 (2012)). Interestingly, previous data suggests that TRPV4 and not PC2 is the main ciliary mechanosensitive ion channel over short periods of mechanical stimulation (Lee, K. L. et al. Cilia 4, 7 (2015)). TRPV4 and PC2, however, may form a heteromeric channel within the cilium suggesting that their function may in fact be linked, warranting further investigation into their long term ciliary mechanosensing function (Kottgen, M. et al. J. Cell Biol. 182, 437-47 (2008)). The data described herein also demonstrate that enhancing osteocyte primary cilia-mediated mechanosensing significantly potentiates pro-osteogenic paracrine signaling to osteoblasts and MSCs. While osteoblasts and MSCs are certainly both mechanosensitive, the current paradigm is that osteocytes are the predominant mechanosensors within bone, directing osteogenic activity of other bone cells (Schaffler, M. B. et al. Calcif. Tissue Int. 94, 5-24 (2014); Owan, I. et al. Am J Physiol 273, C810-5 (1997); Stavenschi, E. et al. J. Biomech. 55, 99-106 (2017)).

### Example 6: Targeting Primary Cilia to Promote Load-Induced Bone Formation In Vivo

Additional details related to Example 2 and Example 3 are discussed in this Example.

Bone actively responds to mechanical stimuli, yet none of the current treatment options for low bone mass and osteoporosis leverage the inherent mechanosensitivity of bone. The primary cilium has been identified as a critical mechanosensor within bone, and pharmacologically targeting the primary cilium with fenoldopam can enhance osteocyte mechanosensitivity. As described herein, potentiating osteocyte mechanosensing with fenoldopam in vitro promotes pro-osteogenic paracrine signaling to activate osteoblasts and stimulate osteogenic differentiation of mesenchymal stem cells. In this Example an in vivo model of load-induced bone formation was utilized to demonstrate that fenoldopam treatment sensitizes bone to mechanical stimulation, in a dose-dependent manner. Minimal adverse effects of this treatment were examined, as assessed by bone quality, and kidney and liver morphology and histology. Finally, it is demonstrated that fenoldopam treatment can augment load-induced bone formation in osteoporotic bones, using a mouse model of postmenopausal osteoporosis. This Example is the first to examine the efficacy of targeting primary cilia-mediated mechanosensing to treat osteoporotic bones.

As previously described herein, osteoporosis is a devastating condition that contributes to increased risk of fracture and extended hospitalization, with over 50% of the US population over 50 years old having low bone mass leading to osteoporosis (US Department of Health and Human Services. Bone health and osteoporosis: a report of the Surgeon General. US Health and Human Services (2004). doi:10.2165/00002018-200932030-00004). It has long been known that bone actively responds to mechanical stimulation, with load being a potent anabolic stimulus of bone formation (Rubin, C. T. & Lanyon, L. E. J. Orthop. Res. 5, 300-310 (1987)). Despite this, none of the current osteoporosis therapeutics leverage the inherent mechanosensitivity of bone. Bisphosphonates and RANKL inhibitors are antiresorptive agents that mitigate bone resorption, while the anti-sclerostin antibody prevents inactivation of bone-forming osteoblasts (Shah, A., et al. Int. J. Womens. Health 7, 565-580 (2015); Deal, C. Nat. Clin. Pract. Rheumatol. 5, 20-27 (2009)). Furthermore, these compounds are starting to be met with patient concern of increased microdamage with reports of mandibular necrosis and atypical fracture (Black, D. M. et al. N. Engl. J. Med. 362, 1761-71 (2010); Marx, R. E. J. Oral Maxillofac. Surg. 61, 1115-7 (2003)). Thus, the need for a new class of osteoporosis therapeutics is growing.

Bone is known to actively respond to mechanical stimulation, with load being a potent anabolic stimulus of bone formation. Previous work has demonstrated that primary cilia play a key role in whole bone mechanosensitivity. When primary cilia are removed from osteocytes and osteoblasts using a DMP1-CRE Kif3A knockout mouse model, the animals display significantly attenuated load-induced bone adaptation (Temiyasathit, S. et al. PLoS One 7, (2012)). Furthermore, mice lacking the cilia-localized protein, adenylyl cyclase 6 (AC6) also display significantly diminished load-induced bone formation (Lee, K. L. et al. FASEB J. 28, 1157-1165 (2014)). Interestingly, these global AC6 knockout animals display no alteration in normal bone development, suggesting a key role of AC6 in the response specifically to heightened mechanical stimuli.

As described herein, primary cilia can be pharmacologically targeted to manipulate cellular mechanotransduction. It is thought that increasing primary cilia length increases the amount of ciliary membrane strain in response to mechanical stimulation, to further stimulate stretch-activated ion channels (Spasic, M. & Jacobs, C. R. Eur. Cell. Mater. 33, 158-168 (2017)). However, it is also likely that increasing primary cilia length promotes production of ciliary proteins to potentiate mechanotransduction events. Dopamine-like 1 receptors localize to the primary cilium, and treatment with the agonist, fenoldopam, increases primary cilia length in endothelial cells, kidney epithelial cells, and osteocytes (Kathem, S. H. et al. J. Geriatr. Cardiol. 11, 63-73 (2014); Upadhyay, V. S. et al. Front. Physiol. 5, 72 (2014); Spasic, M. & Jacobs, C. R. Eur. Cell. Mater. 33, 158-168 (2017)). Intravenous injection of fenoldopam lowers blood pressure in hypertensive patients and mice, by increasing flow-induced nitric oxide production (Kathem, S. H. et al. J. Geriatr. Cardiol. 11, 63-73 (2014)). Furthermore, as described herein, increasing primary cilia length with fenoldopam significantly enhances osteocyte mechanosensitivity and osteogenic signaling (Spasic, M. & Jacobs, C. R. Eur. Cell. Mater. 33, 158-168 (2017)). Besschetnova et al. found that stimulating the cAMP/PKA signaling pathway increases primary cilia length, in an adenylyl cyclase-mediated manner (Besschetnova, T. Y. et al. Curr. Biol. 20, 182-7 (2010)). It has also been described that fenoldopam treatment increases adenylyl cyclase 6 production in human renal proximal tubule cells (Yu, P. et al. Cell. Signal. 26, 2521-2529 (2014)).

Ovariectomized mice (OVX) are a commonly used estrogen depletion model simulating post-menopausal osteoporosis (Klein-Nulend, J., et al. J. Biomech. 1-11 (2014); Bouxsein, M. L. et al. J. Bone Miner. Res. 20, 1085-1092 (2005)). OVX animals display significant changes in bone architecture such as decreased cortical thickness, decreased bone mineral density (BMD), and increased marrow area in cortical bone; as well as decreased bone volume fraction, trabecular number and thickness, and increased trabecular spacing in trabecular bone (Bouxsein, M. L. et al. J. Bone Miner. Res. 20, 1085-1092 (2005)). Four weeks post-ovariectomy in skeletally mature mice has been demonstrated to elicit an osteoporotic phenotype characterized by decreased bone volume fraction, trabecular spacing, trabecular number, and bone mineral density in trabecular bone; and decreased cortical thickness, bone mineral density, and increased marrow area in cortical bone (Li, H. et al. J. Biomech. 46, 1242-1247 (2013)). It has also been suggested that ovariectomy may lead to reduced mechanosensitivity of osteocytes (Klein-Nulend, J., et al. J. Biomech. 1-11 (2014)).

To study the potential of fenoldopam for use in promoting bone formation, a mouse model of compressive ulnar load to stimulate load-induced bone adaptation was utilized. Mice were subcutaneously injected with fenoldopam and subjected to mechanical stimulation. Periosteal bone formation was quantified in response to indicate drug-induced changes in whole bone mechanosensitivity. Potential adverse effects of drug treatment were also examined.

The following materials and methods were used.

Animals and Injections.

16 week old, skeletally mature, C57Bl/6 mice were injected subcutaneously with 20 or 50 mg/kg fenoldopam (US Pharmacopeal Convention), or vehicle control, for 7 consecutive days. Mice were housed in the Columbia University Barrier Facility and fed ad libidum. All procedures performed were in accordance with Columbia University Institutional Animal Care and Use Committee guidelines.

Mechanical Stimulation, In Vivo.

On the final three days of drug or control injection, animals were also subjected to compressive ulnar load at a peak load of 3 N using a 2 Hz sine wave for 120 cycles, as previously described (Temiyasathit, S. et al. PLoS One 7, (2012); Lee, K. L. et al. FASEB J. 28, 1157-1165 (2014)). Contralateral limbs served as a non-loaded control. 2 days following load, mice were treated with 10 mg/kg calcein (Sigma), and 4 days later 70 mg/kg alizarin (Sigma), with mice being sacrificed 6 days later.

Dynamic Histomorphometry.

Dynamic histomorphometry was performed as previously described, measuring the amount and separation of calcein and alizarin labels to quantify mineralizing surface (rMS/BS), mineral apposition rate (rMAR), and bone formation rate (rBFR/BS) relative to non-loaded controls (Temiyasathit, S. et al. PLoS One 7, (2012); Lee, K. L. et al. FASEB J. 28, 1157-1165 (2014)). Left and right ulnae were dissected and preserved in ethanol. Samples were then infiltrated with methyl methacrylate, and embedded in methyl methacrylate and benzoyl peroxide. Transverse sections of the ulnar midshaft were imaged on a laser scanning confocal microscope (Olympus Fluoview FV1000). Measurements of outer bone perimeter (OP), single label perimeter (SL), double label perimeter (DL), and double label area (DA) were made in ImageJ. The mineralizing surface relative to outer bone surface (MS/BS), mineral apposition rate (MAR), and bone formation rate (BFS/BS) were calculated as:

μm per day; μm³/μm²per year

Relative measurements—rMS/BS, rMAR, rBFR/BS—were determined by subtracting non-loaded from loaded ulnae to display differences due to mechanical load.

mRNA Expression.

For gene expression analysis, mice underwent the first 5 days of fenoldopam or vehicle injection, and 1 day of ulnar load. 24 hrs later mice were sacrificed, and ulnae were dissected, flash-frozen, and pulverized as previously described (Robling, A. G. et al. J Biol Chem 283, 5866-5875 (2008)). Total mRNA was converted to cDNA by TaqMan reverse transcriptase (Applied Biosystems). Gene expression was analyzed by quantitative real-time PCR using primers and probes (Life Technologies) for analysis of adenylyl cyclase 6, Adcy6 (Mm00475772_m1), and GAPDH (4351309). Samples and standards were run in triplicate, and all gene expression was normalized to GAPDH endogenous control, as previously performed (Spasic, M. & Jacobs, C. R. Eur. Cell. Mater. 33, 158-168 (2017)).

Osteoporotic Animals.

Ovariectomized (OVX) and Sham animals were purchased directly from Jackson Laboratories, with surgery performed at 12 weeks old. Mice followed the same injection (20 mg/kg fenoldopam or vehicle control) and loading timepoints described above beginning at 16 weeks old, a timeline previously demonstrated to exhibit an osteoporotic phenotype in mouse ulnae (Li, H. et al. J. Biomech. 46, 1242-1247 (2013)). Dynamic histomorphometry was only performed on OVX animals, while Sham animals were used to confirm osteoporotic phenotype based on μCT analysis.

μCt Analysis.

Non-loaded ulnae were used for μCT analysis. Samples were imaged with a Scanco ViaCT 80 at 10.5 μm isotropic resolution. Images were processed using a Gaussian filter and global threshold to segment bone volumes. Ulnar midshafts were imaged and used to assess cortical bone, while the proximal epiphysis was used to examine ulnar trabecular bone. Cortical bone analysis was performed to determine total area, cortical area, marrow area, bone volume fraction (BV/TV), cortical thickness, moment of inertia (Iₘₐₓ and Iₘᵢₙ), and bone mineral density (BMD). Trabecular analyses included BV/TV, connectivity density, trabecular number, trabecular thickness, trabecular spacing, and BMD.

Kidney/Liver Analysis.

Kidneys and liver were dissected from animals, weighed, fixed in 10% formalin, and stored in 70% ethanol before being paraffin embedded. 5 μm thick longitudinal sections were made, and samples were stained with hematoxylin and eosin to visualize tissue structure. Images were captured at 4× magnification. Kidney function was broadly examined by urine creatinine concentration. Mice were subcutaneously injected with 20 mg/kg fenoldopam 3 days per week (Monday, Wednesday, Friday) for 5 weeks, with urine collected once per week. Hydrophobic sand (Lab Sand) was used for urine sample collection, and analysis was performed using a total urine creatinine kit (Cayman Chemical).

Analysis.

All data were analyzed with one-way ANOVA followed by Bonferroni post-hoc correction. Dynamic histomorphometry data were initially analyzed with 2-way ANOVA to determine no sex-based differences in drug-enhanced bone adaptation. Values are reported as mean±SEM, with p<0.05 considered statistically significant.

Sample size, n, represents biological replicates.

Results:

Fenoldopam Enhances Load-Induced Bone Formation.

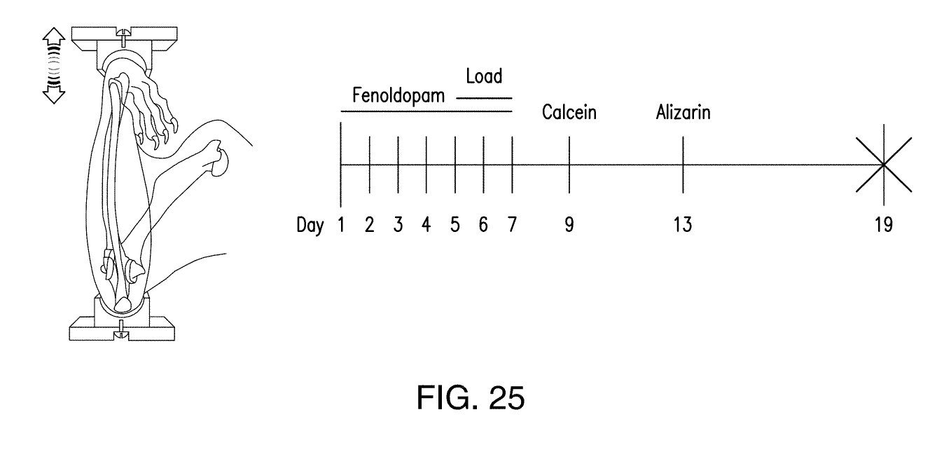

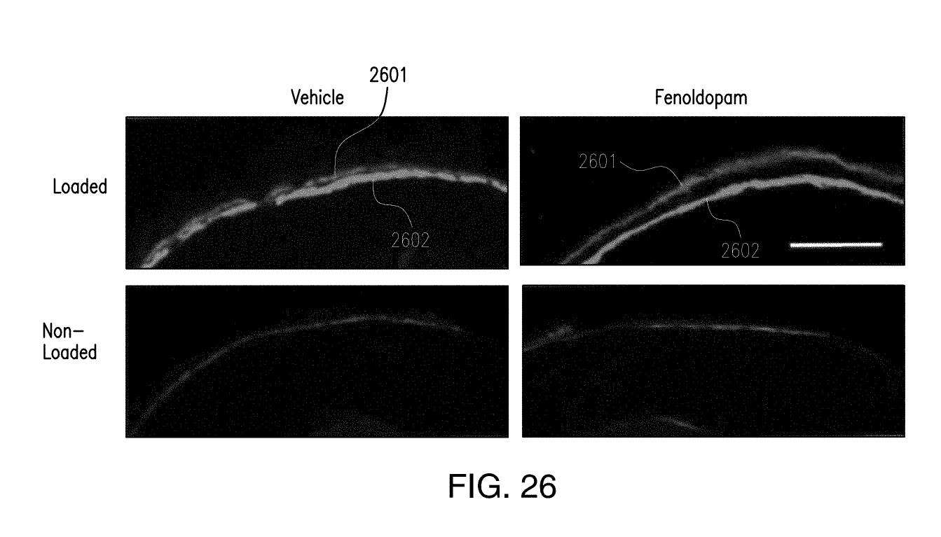

First, translation of the in vitro findings into an in vivo model of load-induced bone formation was investigated. Skeletally mature, 16 week old, mice were subcutaneously injected with fenoldopam (20 mg/kg or 50 mg/kg) or vehicle control for 7 consecutive days, as shown in FIG. 25. On the final 3 days, mice were also subjected to compressive ulnar load, while contralateral limbs served as non-loaded controls, as previously performed (Lee, K. L. et al. FASEB J. 28, 1157-1165 (2014)). Animals were then treated with calcein and alizarin 4 days apart to allow quantification of periosteal bone formation by standard dynamic histomorphometry (Temiyasathit, S. et al. PLoS One 7, (2012); Lee, K. L. et al. FASEB J. 28, 1157-1165 (2014)), as shown in FIG. 26. In FIG. 26, Calcein (indicated by 2602) and alizarin (indicated by 2601) labels were injected 4 days apart. Greater amounts of labeling around the periosteal surface of bone indicate increased mineralizing surface, while separation of labels indicates mineral apposition. Qualitatively, load greatly enhances bone formation, and fenoldopam treatment increases label separation. Scale bar in FIG. 26 represents 100 μm. The relative amount of mineralizing surface (rMS/BS) was quantified as the amount of labeled surface normalize to the bone surface. The mineral apposition rate (rMAR) is quantified by the separation of calcein and alizarin labels, while the bone formation rate (rBFR/BS) is the product of rMS/BS and rMAR. While neither fenoldopam concentration elicited a significant increase in rMS/BS, 20 mg/kg fenoldopam results in a significant increase in rMAR and rBFR/BS compared to vehicle control (FIG. 26). Interestingly, the higher dose of fenoldopam resulted in no statistically significant difference in any of these parameters.

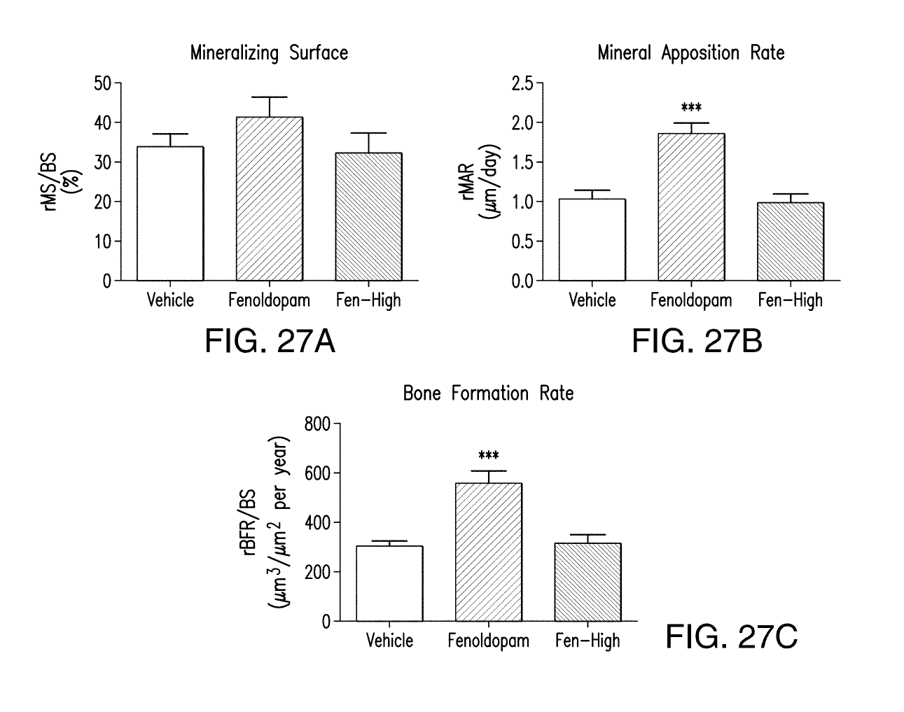

As shown in FIG. 27, Mice were treated with fenoldopam (20 mg/kg, or “Fen-High” at 50 mg/kg) for 7 consecutive days and also subjected to compressive ulnar load on the final 3 days. Fenoldopam treatment displayed no effect in enhancing the amount of mineralizing surface. However, the lower dose of fenoldopam (20 mg/kg) elicited significant increases in load-induced mineral apposition rate and bone formation rate. The higher dose of fenoldopam (50 mg/kg) had no effect on load-induced bone adaptation. Mean±SEM; n=26, 12, 14 for each group, respectively; ***p<0.001. Male and female animals were grouped as 2-way ANOVA revealed no sex-based differences. All data are reported relative to non-loaded contralateral limbs.

AC6 mRNA Expression in Ulnae.

As described herein, adenylyl cyclase 6 was identified as a critical signal transduction protein in load-induced bone adaptation. Mice injected with 20 mg/kg fenoldopam for 5 days were sacrificed, and their ulnae were dissected for analysis of AC6 mRNA expression. In fact, the effective dose of fenoldopam that enhances load-induced bone formation also elicited a significant increase in AC6 mRNA expression.

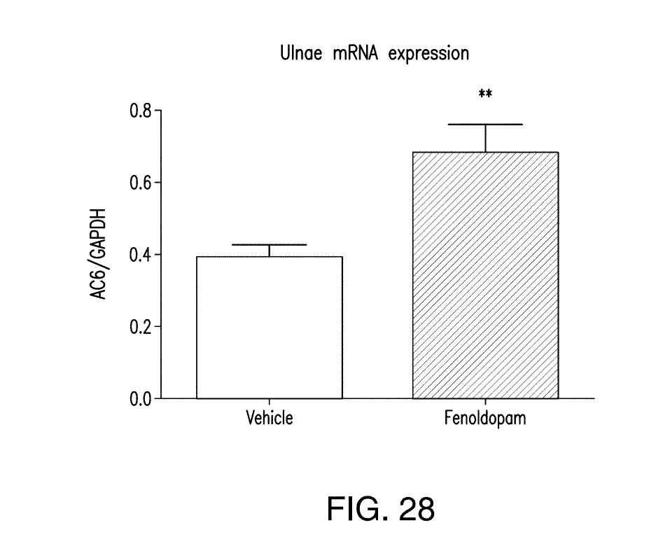

Mice were treated with fenoldopam (20 mg/kg) for 5 days. 24 hours following the final injection mice were sacrificed and ulnae were dissected, flash frozen, and pulverized for mRNA analysis. As shown in FIG. 28, Fenoldopam treatment increases AC6 mRNA expression in the ulnar cortical bone relative to vehicle control. Mean±SEM; n=8 for each group; **p<0.01.

Minimal Adverse Effects of Fenoldopam Treatment.

In addition to load-induced bone formation, potential indicators of adverse effects of drug treatment were also examined. Loaded ulnae from fenoldopam treated mice were examined for bone ultrastructure and microarchitecture.

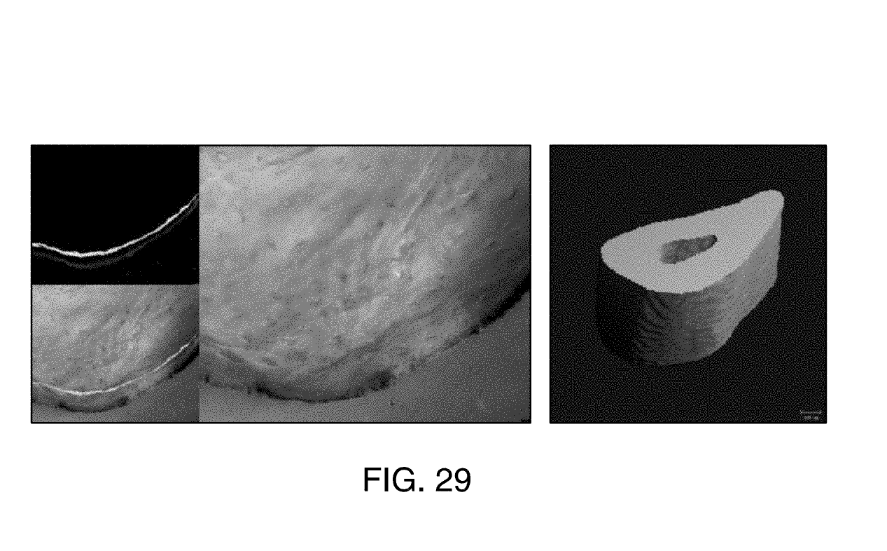

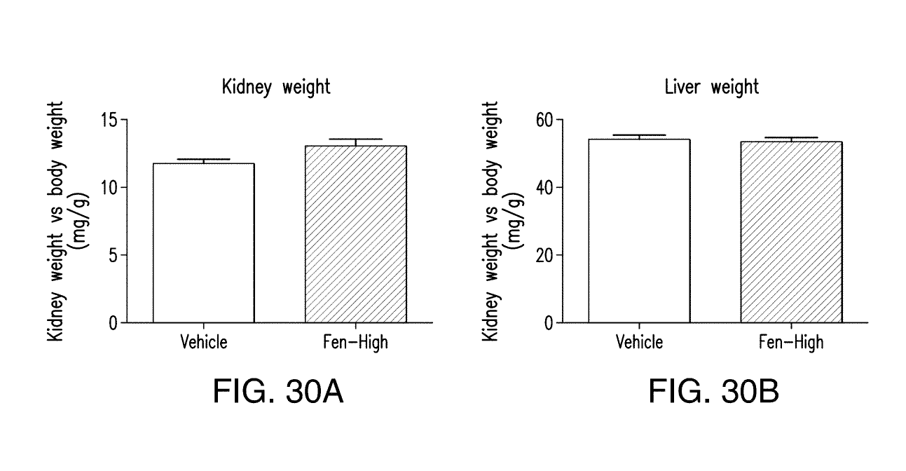

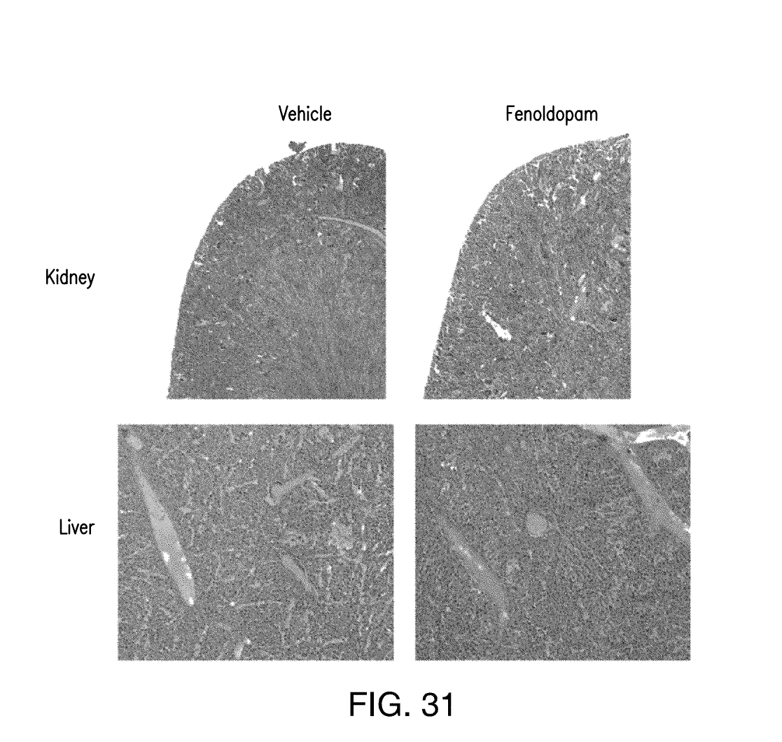

Exemplary images are shown in FIG. 29. As shown in FIG. 29, left panels, qualitative examination of the mid diaphysis of loaded ulnae suggest no changes in the quality of bone formed. Fenoldopam treatment enhanced load-induced bone formation, without promoting woven bone formation. As shown in FIG. 29, right panel, μCT analysis was performed on non-loaded ulnae to assess potential fenoldopam-induced changes in bone architecture. These results are summarized in Table 5. Fenoldopam treatment elicited no qualitative change in bone ultrastructure, suggesting no difference in woven versus lamellar bone formed in response to load (FIG. 29). Additionally, μCT analysis revealed no difference in normal bone microarchitecture due to drug treatment (FIG. 29, Table 5). μCT was performed on ulna midshaft to examine cortical bone at the region where dynamic histomorphometric measurements were taken. No changes in bone architecture were examined in 20 mg/kg fenoldopam treated animals, compared to vehicle control (Table 5). Kidney and liver weight was also assessed as an indicator of tissue inflammation, with no change examined relative to vehicle control (FIG. 30). The kidneys and liver were dissected from fenoldopam (50 mg/kg) treated animals and weighed relative to total body weight. Even at the higher dose of fenoldopam we see no change in kidney or liver weight. Mean±SEM; n=10, 14 for each vehicle and control, respectively. Furthermore, no changes kidney and liver structure were observed, as assessed by H&E. FIG. 31 is a set of images showing exemplary histology results of kidney and liver from mice treated with fenoldopam or vehicle. Kidneys and livers dissected from fenoldopam (50 mg/kg) treated animals were fixed, paraffin embedded, sectioned, and stained with H&E. The higher dose of fenoldopam does not cause any changes in tissue morphology compared to vehicle control.

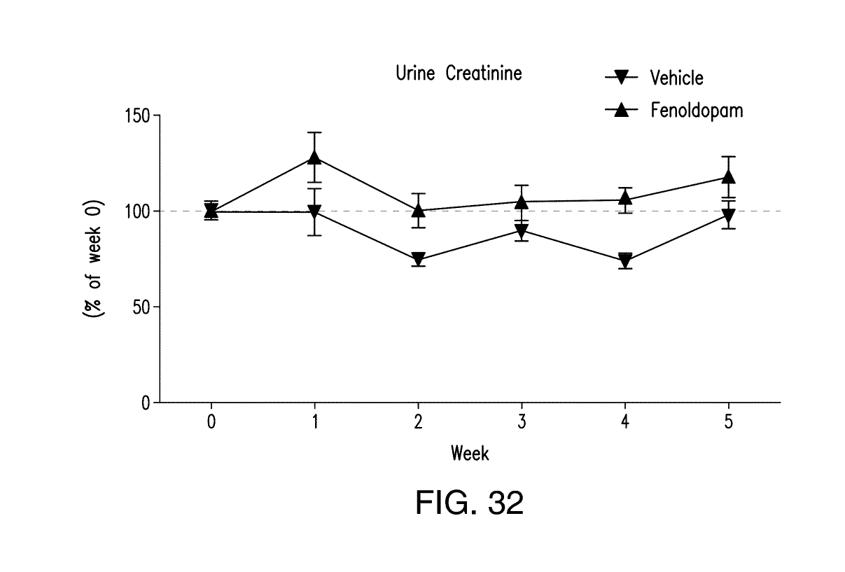

Urine creatinine was also examined over long-term drug treatment—3 days per week with 20 mg/kg fenoldopam for 5 weeks—and suggests no change in kidney function. FIG. 32 is a graph reporting exemplary data on urea creatinine in mice treated with fenoldopam or vehicle. Urine creatinine was assessed in animals treated with fenoldopam (20 mg/kg) 3 days per week for 5 weeks. Urine was collected at the same time once per week. After 5 weeks of drug treatment, there is no significant difference in urine creatinine concentration relative to pre-drug treatment values. Mean±SEM; n=7 for each group.

TABLE 5
Short-term fenoldopam treatment does not affect bone architecture.

| Bone and | Female | | Male | |
|---|---|---|---|---|
| parameter | Vehicle | Fenoldopam | Vehicle | Fenoldopam |

Ulnar midshaft - cortical

| | | | | |
|---|---|---|---|---|
| n | 9 | 5 | 8 | 7 |
| Total area | 0.352 ± 0.006 | 0.357 ± 0.005 | 0.380 ± 0.006 | 0.370 ± 0.010 |
| (mm²) | | | | |
| Cortical area | 0.306 ± 0.006 | 0.310 ± 0.003 | 0.330 ± 0.006 | 0.320 ± 0.009 |
| (mm²) | | | | |
| Marrow area | 0.046 ± 0.003 | 0.310 ± 0.003 | 0.051 ± 0.001 | 0.049 ± 0.001 |
| (mm²) | | | | |
| Bone vol./ | 0.869 ± 0.002 | 0.310 ± 0.003 | 0.867 ± 0.001 | 0.866 ± 0.001 |
| Total vol. | | | | |
| Cortical | 0.173 ± 0.003 | 0.173 ± 0.006 | 0.172 ± 0.002 | 0.169 ± 0.004 |
| thickness | | | | |
| (mm) | | | | |
| Iₘₐₓ (mm⁴) | 0.028 ± 0.001 | 0.029 ± 0.001 | 0.037 ± 0.002 | 0.033 ± 0.003 |
| Iₘᵢₙ (mm⁴) | 0.004 ± 0.001 | 0.005 ± 0.000 | 0.005 ± 0.001 | 0.004 ± 0.001 |
| Bone Mineral | 1236.4 ± 5.0 | 1224.143 ± 11.750 | 1234.7 ± 9.1 | 1218.5 ± 7.7 |
| Density | | | | |
| (mg/mm³) | | | | |

The ulnar midshaft of non-loaded animals treated with 20 mg/kg fenoldopam was examined by μCT. Standard cortical bone parameters were examined. Fenoldopam treatment did not result in any change in bone architecture at the region where dynamic histomorphometry was performed.

Fenoldopam is Efficacious in Osteoporotic Animals.

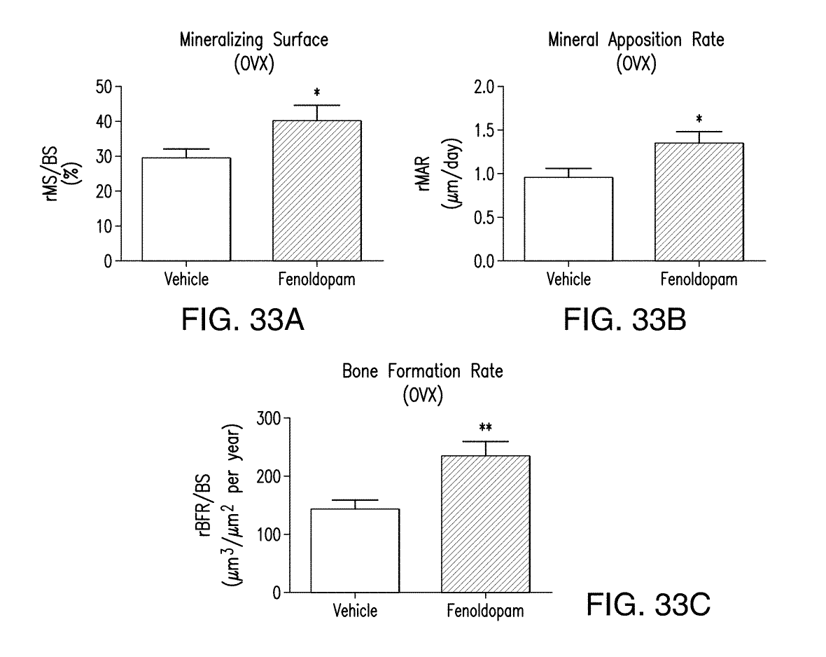

Next, ovariectomized (OVX) mice were used as an estrogen-deficiency model of post-menopausal osteoporosis. Mice underwent OVX surgery at 12 weeks old. 4 weeks post surgery mice were injected with 20 mg/kg fenoldopam for 7 days and subjected to compressive ulnar load. Fenoldopam treated animals displayed significantly augmented load-induced bone adaptation as measured by increased rMS/BS, rMAR, and rBFR/BS compared to vehicle control. FIG. 33 is a set of graphs reporting exemplary data on mineralizing surface (Panel A), mineral apposition rate (Panel B) and bone formation rate (Panel C) in osteoporotic mice treated with vehicle or fenoldopam. Ovariectomy (OVX) was used as a model of post-menopausal osteoporosis. Mice were subjected to OVX surgery at 12 weeks old, and fenoldopam (20 mg/kg) injections began at 16 weeks old. In osteoporotic animals, fenoldopam significantly enhances load-induced bone adaptation as quantified by dynamic histomorphometry. Mean±SEM; n=10 for each group; *p<0.05, **p<0.01.

μCT was performed on ulnar cortical (mid diaphysis) and trabecular (proximal epiphysis) bone to confirm effects of OVX on ulnae microarchitecture. Results are shown in Table 6.

TABLE 6
μCT analysis of osteoporotic and sham fenoldopam treated animals.

| Bone and | Sham | | OVX | |
|---|---|---|---|---|
| parameter | Vehicle | Fenoldopam | Vehicle | Fenoldopam |

Ulnar midshaft -cortical

| | | | | |
|---|---|---|---|---|
| n | 6 | 6 | 10 | 10 |
| Total area | 0.310 ± 0.009 | 0.322 ± 0.009 | 0.319 ± 0.004 | 0.307 ± 0.005 |
| (mm²) | | | | |
| Cortical area | 0.268 ± 0.007 | 0.276 ± 0.008 | 0.272 ± 0.003 | 0.261 ± 0.005 |
| (mm²) | | | | |
| Marrow area | 0.043 ± 0.001+ | 0.046 ± 0.001 | 0.047 ± 0.001+ | 0.046 ± 0.001 |
| (mm²) | | | | |
| Bone vol./ | 0.863 ± 0.002* | 0.859 ± 0.002* | 0.853 ± 0.001* | 0.849 ± 0.003* |
| Total vol. | | | | |
| Cortical | 0.162 ± 0.001* | 0.162 ± 0.001* | 0.154 ± 0.002* | 0.151 ± 0.003* |
| thickness | | | | |
| (mm) | | | | |
| Iₘₐₓ (mm⁴) | 0.016 ± 0.002 | 0.019 ± 0.002 | 0.019 ± 0.001 | 0.018 ± 0.001 |
| Iₘᵢₙ (mm⁴) | 0.005 ± 0.001 | 0.005 ± 0.001 | 0.005 ± 0.001 | 0.004 ± 0.001 |
| Bone Mineral | 1097.4 ± 3.8* | 1089.5 ± 5.7 | 1082.5 ± 2.2* | 1079.5 ± 3.9 |
| Density | | | | |
| (mg/mm³) | | | | |

Ulnar proximal epiphysis - trabecular

| | | | | |
|---|---|---|---|---|
| Bone vol./ | 0.175 ± 0.008* | 0.183 ± 0.008* | 0.136 ± 0.004* | 0.130 ± 0.008* |
| Total vol. | | | | |
| Conn. Dens | 233.7 ± 17.6 | 232.8 ± 22.6 | 202.9 ± 14.3 | 187.6 ± 20.0 |
| (mm⁻³) | | | | |
| SMI | 1.309 ± 0.067+ | 1.362 ± 0.083+ | 1.736 ± 0.045+ | 1.807 ± 0.067+ |
| Tb. Number | 5.201 ± 0.123 | 5.384 ± 0.159* | 4.928 ± 0.114 | 4.853 ± 0.139* |
| (mm⁻¹) | | | | |
| Tb. Thickness | 0.0434 ± 0.0015 | 0.0445 ± 0.0015* | 0.0401 ± 0.0007 | 0.0395 ± 0.0010* |
| (mm) | | | | |
| Tb. Spacing | 0.192 ± 0.005 | 0.182 ± 0.005+ | 0.206 ± 0.005 | 0.208 ± 0.006+ |
| (mm) | | | | |
| Bone Mineral | 257.9 ± 10.5* | 268.8 ± 10.3* | 208.2 ± 7.1* | 201.9 ± 10.2* |
| Density | | | | |
| (mg/mm³) | | | | |

Non-loaded ulnae from OVX and Sham control animals were examined by μCT to assess bone microarchitecture. Statistical analyses were performed between OVX-Sham for either vehicle or fenoldopam treatment. In Table 6, (*) denotes values where OVX is significantly smaller compared to Sham counterpart. (+) denotes values where OVX is significantly higher compared to Sham counterpart. Data demonstrate that at this timepoint OVX animals display an osteoporotic phenotype with significantly lower bone volume fraction (bone vol./total vol.) and bone mineral density, with increased marrow area in cortical bone. In trabecular bone OVX animals display significantly decreased bone volume fraction and bone mineral density, with decreased trabecular number and thickness, and increased trabecular spacing and structure model index (SMI). Mean±SEM; */+p<0.05.

The results in this Example demonstrate that targeting primary cilia-mediated mechanosensing with fenoldopam treatment significantly enhances load-induced bone formation. While fenoldopam is clinically used for hypertension, this is the first work studying the effects of this treatment on bone in vivo.

The loading data suggests that short-term fenoldopam treatment does not affect normal bone architecture. μCT analysis of non-loaded cortical bone demonstrates no change in bone physical properties after 7 days of fenoldopam injection. This treatment does, however, significantly enhance load-induced bone adaptation. Together this suggests that short-term fenoldopam treatment sensitizes cells to mechanical stimulation rather than eliciting long-term systemic changes in bone quality. This type of treatment could be used to sensitize bone to lower amounts of load, thus preventing progression of limited use-induced bone loss. Potential clinical application of fenoldopam to enhance bone formation would likely require an associated exercise regimen.

It is not yet completely defined how fenoldopam treatment enhances primary cilia-mediated mechanotransduction in bone, and it is likely a combination of both structural and biochemical changes. While in vitro significant increases in cilia length were identified, the limited lacunar space in which osteocytes reside may impede substantial cilia elongation (Spasic, M. & Jacobs, C. R. Eur. Cell. Mater. 33, 158-168 (2017)). Computational models have suggested that specific orientations of primary cilia may allow for elongation leading to enhanced mechanosensitivity (Vaughan, T. J., et al. Biomech. Model. Mechanobiol. 2, (2014)). This model also posits that osteocyte primary cilia may not be free standing organelles in vitro and may anchor to the lacunar wall, in a manner similar to chondrocyte cilia attaching to the extracellular matrix (McGlashan, S. R. et al. J. Histochem. Cytochem. 54, 1005-1014 (2006)). Regardless of the means of physical changes to primary cilia structure, the data described herein also demonstrates that fenoldopam treatment significantly enhances AC6 production. As previously described, recent work indicates that deletion of AC6 impairs load-induced bone formation, suggesting that the opposite may also be true, and that increasing AC6 production will augment load-induced bone formation (Lee, K. L. et al. FASEB J. 28, 1157-1165 (2014)). Together the increase in cilia length, enabling further stimulation of stretch-activated ion channels, and enhanced AC6 protein levels potentiate bone mechanosensitivity.

There is a therapeutic window in which fenoldopam treatment is effective in promoting load-induced bone formation. While 20 mg/kg fenoldopam significantly enhanced bone adaptation, 50 mg/kg elicited no bone formation response. Previous work identified that fenoldopam treatment increases primary cilia length in a dose-dependent manner (Kathem, S. H. et al. J. Geriatr. Cardiol. 11, 63-73 (2014)). Moreover, when the concentration was increased to 2.5× the peak effective dose, cilia length reduces closer to baseline level. Currently it is not completely evident why this dose-dependence occurs, but it is likely due to toxicity. During injections with the higher dose of fenoldopam, a small amount of tissue necrosis was observed near the injection site in several animals, which was not observed in mice treated with the effective, 20 mg/kg, dose. While this high dose may be associated with some level of toxicity, changes in kidney or liver weight and morphology were not observed. Further toxicology would need to be performed to fully characterize adverse events.

Fenoldopam does not specifically target osteocytes, thus allowing potential for adverse effects. While fenoldopam is clinically used to treat extreme hypertension, it resembles other antihypertensive compounds and has been shown to have limited or no effect hypotensive effect in normotensive patients (Murphy, M. B. et al. Clin. Pharmacol. Ther. 44, 49-55 (1988); MacGregor, G. A. et al. J. Cardiovasc. Pharmacol. 4 Suppl 3, S358-62 (1982)). Furthermore, low enough doses of fenoldopam can still increase endothelial cilia length, and mechanically induced nitric oxide production, without affecting blood pressure (Kathem, S. H. et al. J. Geriatr. Cardiol. 11, 63-73 (2014)). Fenoldopam treatment has, however, also been implicated with an increase in renal blood flow and an increase in natriuresis, diuresis, and glomerular filtration rate, potentially all leading to hypertensive renal damage and kidney disease (Elliott, W. J. et al. Circulation 81, 970-977 (1990)). High enough doses of fenoldopam, 100 mg/kg, have also been found to be vasotoxic in a rat model (Dalmas, D. A. et al. Toxicol. Pathol. 36, 496-519 (2008)). The effective, 20 mg/kg, treatment dose did not elicit any change in urine creatinine concentration, and even at the higher dose, 50 mg/kg, there was no change in kidney and liver weight or morphology. Fenoldopam also does not cross the blood brain barrier, so there is minimal risk of dopaminergic central nervous system effects resulting from treatment (Stote, R. M. et al. Clin. Pharmacol. Ther. 34, 309-15 (1983)). While primary cilia are not unique to osteocytes and fenoldopam does not specifically target bone, the data described herein suggests minimal adverse systemic affects while still enhancing bone mechanosensitivity. A cell-type specific therapy would mitigate any concerns of adverse effects.

Together, these data demonstrate that targeting the primary cilium is a potent strategy for directing cell mechanotransduction, and intercellular communication to dictate whole tissue function. This is the first demonstration of a ciliotherapy enhancing bone adaptation in vivo and, moreover, enhancing load-induced bone formation in osteoporotic subjects. Fenoldopam is clinically used, but until now has never been examined in the context of whole bone adaptation. The results described herein suggest that there is significant potential for repurposing fenoldopam for use in bone indications, and for developing cilia-targeted therapeutics to direct cell and tissue mechanotransduction to combat human diseases.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

We claim:

1. A method of increasing expression of a gene in a cell comprising:
contacting the cell with an effective amount of a cilium elongation modulator,
wherein the cilium elongation modulator increases a length of one or more primary cilia of the cell.

2. The method of claim 1, wherein the cilium elongation modulator is contacted to the cell in an amount effective to increase mechanosensitivity of the cell.

3. The method of claim 1, wherein the cilium elongation modulator is contacted to the cell in an amount effective to increase cAMP level in the cell.

4. The method of claim 3, wherein the cilium elongation modulator is contacted to the cell in an amount effective to increase expression level or an enzymatic activity of adenylyl cyclase.

5. The method of claim 1, wherein the cilium elongation modulator comprises a dopamine D1-like receptor agonist, derivatives thereof, and combinations thereof.

6. The method of claim 5, wherein the cilium elongation modulator is selected from the group consisting of fenoldopam, Dihydrexidine (CAS No: 158704-02-0), Dopamine (CAS No: 62-31-7), NPEC-caged-dopamine (CAS No: 1257326-23-0), SKF 38393 (CAS No: 20012-10-6), SKF 77434 (CAS No: 300561-58-4), SKF 81297 (CAS No: 67287-39-2), SKF 82958 (CAS No: 74115-01-8), SKF 83822 (CAS No: 74115-10-9), SCH-23390 (CAS No: 87075-17-0), SKF-83959 (CAS No: 67287-95-0), A68930 (CAS No: 130465-39-3), A77636 (CAS No: 145307-34-2), (R)-(−)-Apomorphine (CAS No: 314-19-2), CY 208-243 (CAS No: 100999-26-6), Ecopipam (SCH-39,166, CAS No: 112108-01-7), derivatives thereof, and combinations thereof.

7. The method of claim 5, wherein the dopamine D1-like receptor agonist is selected from the group consisting of fenoldopam, derivatives thereof, and combinations thereof.

8. The method of claim 1, wherein the cilium elongation modulator is selected from the group consisting of lithium, derivatives thereof, and combinations thereof.

9. The method of claim 1, wherein the cilium elongation modulator comprises an adenylyl cyclase agonist, derivatives thereof, and combinations thereof.

10. The method of claim 9, wherein the cilium elongation modulator is selected from the group consisting of fenoldopam, forskolin, NKH 477 (CAS No: 138605-00-2), PACAP 1-27 (CAS No: 127317-03-7), PACAP 1-38 (137061-48-4), derivatives thereof, and combinations thereof.

11. The method of claim 1, wherein the cillium elongation modulator increases the expression of one or more gene selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

12. The method of claim 1, wherein the cell is an osteocyte, an osteoblast, an osteoclast, an osteoprogenitor cell, or a combination thereof.

13. The method of claim 1, wherein the cillium elongation modulator is contacted to a population of cells.

14. The method of claim 1, wherein the gene is an osteogenic gene.

15. The method of claim 14, wherein the osteogenic gene is selected from the group consisting of COX-2, OPN, BSP, Collagen I, Osteocalcin, Runt-related transcription factor 2 (Runx2, (Cbfa1/PEBP2αA/AML-3/Osf2)), Osterix (Osx), distal-less homeobox protein 5 (Dlx5), Alkaline phosphatase (ALP), Msx-2 (Hox-8), Nuclear factor of kappa light polypeptide gene enhancer in B-cells (NF-κB), Osteoprotegerin (OPG), Cytochrome c oxidase subunit 2 (Cox-2), fibroblast growth factor 2 (FGF2), bagpipe homeobox homolog 1 (Drosophila) (Bapx1), Collagen I, Osteocalcin, Osteopontin (OPN), Bone sialoprotein (BSP), AHSG, AMBN, AMELY, BGLAP, ENAM, MINPP1, STATH, TUFT1, Cartilage condensation genes: BMP1, COL11A1, SOX9, ALPL, AMBN, AMELY, BGLAP, CALCR, CDH11, DMP1, DSPP, ENAM, MINPP1, PHEX, RUNX2, STATH, TFIP11, TUFT1, ANXA5, BGLAP, BMP1, CALCR, CDH11, COMP, DMP1, EGF, MMP2, MMP8, VDR, BMP1, BMP2, BMP3, BMP4, BMP5, BMP6, CSF2, CSF3, EGF, EGFR, FGF1, FGF2, FGF3, FGFR1, FGFR2, FLT1, GDF10, IGF1, IGF1R, IGF2, PDGFA, TGFB1, TGFB2, TGFB3, TGFBR1, TGFBR2, VEGFA, VEGFB, COL4A3, COL10A1, COL11A1, COL12A1, COL14A1, COL15A1, COL1A1, COL1A2, COL2A1, COL3A1, COL4A3, COL5A1, AHSG, COL4A3, SERPINH1, CTSK, MMP10, MMP2, MMP8, MMP9, and PHEX, CDH11, COL11A1, COL14A1, ICAM1, ITGB1, VCAM1, ITGA1, ITGA2, ITGA3, ITGAM, ITGB1, BGLAP, CD36, COL12A1, COL15A1, COL4A3, COL5A1, COMP, FN1, SCARB1, TNF, MSX1, NFKB1, RUNX2, SMAD1, SMAD2, SMAD3, SMAD4, SOX9, TNF, TWIST1, VDR, and combinations thereof.

16. The method of claim 15, wherein the osteogenic gene is selected from the group consisting of COX-2, OPN, BSP, Collagen I, Osteocalcin, and combinations thereof.

17. The method of claim 14, wherein the cell is comprised in a population of cells, and the cilium elongation modulator is contacted to the population of cells in an amount effective to increase osteogenesis.

18. The method of claim 1, wherein the cell is a mammalian cell.

19. The method of claim 18, wherein the mammalian cell is a human cell.

20. A method of treating a ciliopathy in a subject comprising:
administering to a subject in need thereof, an effective amount of a cilium elongation modulator,
wherein the cilium elongation modulator increases a length of one or more primary cilia of a cell in the subject.

21. The method of claim 20, wherein the cilium elongation modulator is administered to the subject in an amount effective to increase expression of a gene in the cell of the subject.

22. The method of claim 20, wherein the cilium elongation modulator is administered to the subject in an amount effective to increase mechanosensitivity of the cell of the subject.

23. The method of claim 20, wherein the cilium elongation modulator is contacted to the cell in an amount effective to increase cAMP level in the cell.

24. The method of claim 23, wherein the cilium elongation modulator is contacted to the cell in an amount effective to increase expression level or an enzymatic activity of adenylyl cyclase.

25. The method of claim 20, wherein the cilium elongation modulator comprises a dopamine D1-like receptor agonist, derivatives thereof, and combinations thereof.

26. The method of claim 20, wherein the cilium elongation modulator is selected from the group consisting of fenoldopam, Dihydrexidine (CAS No: 158704-02-0), Dopamine (CAS No: 62-31-7), NPEC-caged-dopamine (CAS No: 1257326-23-0), SKF 38393 (CAS No: 20012-10-6), SKF 77434 (CAS No: 300561-58-4), SKF 81297 (CAS No: 67287-39-2), SKF 82958 (CAS No: 74115-01-8), SKF 83822 (CAS No: 74115-10-9), SCH-23390 (CAS No: 87075-17-0), SKF-83959 (CAS No: 67287-95-0), A68930 (CAS No: 130465-39-3), A77636 (CAS No: 145307-34-2), (R)-(−)-Apomorphine (CAS No: 314-19-2), CY 208-243 (CAS No: 100999-26-6), Ecopipam (SCH-39,166, CAS No: 112108-01-7), derivatives thereof, and combinations thereof.

27. The method of claim 26, wherein the dopamine D1-like receptor agonist is selected from the group consisting of fenoldopam, derivatives thereof, and combinations thereof.

28. The method of claim 20, wherein the cilium elongation modulator is selected from the group consisting of lithium, derivatives thereof, and combinations thereof.

29. The method of claim 20, wherein the cilium elongation modulator comprises an adenylyl cyclase agonist, derivatives thereof, and combinations thereof.

30. The method of claim 29, wherein the cilium elongation modulator is selected from the group consisting of fenoldopam, forskolin, NKH 477 (CAS No: 138605-00-2), PACAP 1-27 (CAS No: 127317-03-7), PACAP 1-38 (137061-48-4), derivatives thereof, and combinations thereof.

31. The method of claim 20, wherein the cillium elongation modulator increases the expression of one or more gene selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

32. The method of claim 20, wherein the cell is an osteocyte, an osteoblast, an osteoclast, an osteoprogenitor cell, or a combination thereof.

33. The method of claim 20, wherein the gene is an osteogenic gene.

34. The method of claim 33, wherein the osteogenic gene is selected from the group consisting of COX-2, OPN, BSP, Collagen I, Osteocalcin, Runt-related transcription factor 2 (Runx2, (Cbfa1/PEBP2αA/AML-3/Osf2)), Osterix (Osx), distal-less homeobox protein 5 (Dlx5), Alkaline phosphatase (ALP), Msx-2 (Hox-8), Nuclear factor of kappa light polypeptide gene enhancer in B-cells (NF-κB), Osteoprotegerin (OPG), Cytochrome c oxidase subunit 2 (Cox-2), fibroblast growth factor 2 (FGF2), bagpipe homeobox homolog 1 (Drosophila) (Bapx1), Collagen I, Osteocalcin, Osteopontin (OPN), Bone sialoprotein (BSP), AHSG, AMBN, AMELY, BGLAP, ENAM, MINPP1, STATH, TUFT1, Cartilage condensation genes: BMP1, COL11A1, SOX9, ALPL, AMBN, AMELY, BGLAP, CALCR, CDH11, DMP1, DSPP, ENAM, MINPP1, PHEX, RUNX2, STATH, TFIP11, TUFT1, ANXA5, BGLAP, BMP1, CALCR, CDH11, COMP, DMP1, EGF, MMP2, MMP8, VDR, BMP1, BMP2, BMP3, BMP4, BMP5, BMP6, CSF2, CSF3, EGF, EGFR, FGF1, FGF2, FGF3, FGFR1, FGFR2, FLT1, GDF10, IGF1, IGF1R, IGF2, PDGFA, TGFB1, TGFB2, TGFB3, TGFBR1, TGFBR2, VEGFA, VEGFB, COL4A3, COL10A1, COL11A1, COL12A1, COL14A1, COL15A1, COL1A1, COL1A2, COL2A1, COL3A1, COL4A3, COL5A1, AHSG, COL4A3, SERPINH1, CTSK, MMP10, MMP2, MMP8, MMP9, and PHEX, CDH11, COL11A1, COL14A1, ICAM1, ITGB1, VCAM1, ITGA1, ITGA2, ITGA3, ITGAM, ITGB1, BGLAP, CD36, COL12A1, COL15A1, COL4A3, COL5A1, COMP, FN1, SCARB1, TNF, MSX1, NFKB1, RUNX2, SMAD1, SMAD2, SMAD3, SMAD4, SOX9, TNF, TWIST1, VDR, and combinations thereof.

35. The method of claim 34, wherein the osteogenic gene is selected from the group consisting of COX-2, OPN, BSP, Collagen I, Osteocalcin, and combinations thereof.

36. The method of claim 34, wherein the cell is comprised in a population of cells, and the cilium elongation modulator is contacted to the population of cells in an amount effective to increase osteogenesis.

37. A method of treating osteoporosis in a subject comprising:
administering to a subject in need thereof, an effective amount of a cilium elongation modulator,
wherein the cilium elongation modulator increases a length of one or more primary cilia of a cell in the subject.

38. The method of claim 37, wherein the cilium elongation modulator is administered to the subject in an amount effective to increase expression of a gene in the cell of the subject.

39. The method of claim 37, wherein the cilium elongation modulator is administered to the subject in an amount effective to increase mechanosensitivity of the cell of the subject.

40. The method of claim 37, wherein the cilium elongation modulator is contacted to the cell in an amount effective to increase cAMP level in the cell.

41. The method of claim 40, wherein the cilium elongation modulator is contacted to the cell in an amount effective to increase expression level or an enzymatic activity of adenylyl cyclase.

42. The method of claim 37, wherein the cilium elongation modulator comprises a dopamine D1-like receptor agonist, derivatives thereof, and combinations thereof.

43. The method of claim 37, wherein the cilium elongation modulator is selected from the group consisting of fenoldopam, Dihydrexidine (CAS No: 158704-02-0), Dopamine (CAS No: 62-31-7), NPEC-caged-dopamine (CAS No: 1257326-23-0), SKF 38393 (CAS No: 20012-10-6), SKF 77434 (CAS No: 300561-58-4), SKF 81297 (CAS No: 67287-39-2), SKF 82958 (CAS No: 74115-01-8), SKF 83822 (CAS No: 74115-10-9), SCH-23390 (CAS No: 87075-17-0), SKF-83959 (CAS No: 67287-95-0), A68930 (CAS No: 130465-39-3), A77636 (CAS No: 145307-34-2), (R)-(−)-Apomorphine (CAS No: 314-19-2), CY 208-243 (CAS No: 100999-26-6), Ecopipam (SCH-39,166, CAS No: 112108-01-7), derivatives thereof, and combinations thereof.

44. The method of claim 42, wherein the dopamine D1-like receptor agonist is selected from the group consisting of fenoldopam, derivatives thereof, and combinations thereof.

45. The method of claim 37, wherein the cilium elongation modulator is selected from the group consisting of lithium, derivatives thereof, and combinations thereof.

46. The method of claim 37, wherein the cilium elongation modulator comprises an adenylyl cyclase agonist, derivatives thereof, and combinations thereof.

47. The method of claim 46, wherein the cilium elongation modulator is selected from the group consisting of fenoldopam, forskolin, NKH 477 (CAS No: 138605-00-2), PACAP 1-27 (CAS No: 127317-03-7), PACAP 1-38 (137061-48-4), derivatives thereof, and combinations thereof.

48. The method of claim 37, wherein the cillium elongation modulator increases the expression of one or more gene selected from the group consisting of AKT1, BBS4, CCND1, CDK5RAP2, CDKN1A (P21CIP1/WAF1), IGF1, INS2, MAP2K1, PKD1, PKD2, TRP53. DYNC2LI1, IFT172, IFT20, IFT74, IFT80, TRPV4, IFT88, Kinesin-like protein (KIF3A, KIF3B), ALMS1, ARL6, BBS1, BBS2, BBS4, BBS7, IFT172, IFT88, MKKS, OFD1, PKHD1, RPGRIP1L, VANGL2 and WWTR1.

49. The method of claim 37, wherein the gene is an osteogenic gene.

50. The method of claim 49, wherein the osteogenic gene is selected from the group consisting of COX-2, OPN, BSP, Collagen I, Osteocalcin, Runt-related transcription factor 2 (Runx2, (Cbfa1/PEBP2αA/AML-3/Osf2)), Osterix (Osx), distal-less homeobox protein 5 (Dlx5), Alkaline phosphatase (ALP), Msx-2 (Hox-8), Nuclear factor of kappa light polypeptide gene enhancer in B-cells (NF-κB), Osteoprotegerin (OPG), Cytochrome c oxidase subunit 2 (Cox-2), fibroblast growth factor 2 (FGF2), bagpipe homeobox homolog 1 (Drosophila) (Bapx1), Collagen I, Osteocalcin, Osteopontin (OPN), Bone sialoprotein (BSP), AHSG, AMBN, AMELY, BGLAP, ENAM, MINPP1, STATH, TUFT1, Cartilage condensation genes: BMP1, COL11A1, SOX9, ALPL, AMBN, AMELY, BGLAP, CALCR, CDH11, DMP1, DSPP, ENAM, MINPP1, PHEX, RUNX2, STATH, TFIP11, TUFT1, ANXA5, BGLAP, BMP1, CALCR, CDH11, COMP, DMP1, EGF, MMP2, MMP8, VDR, BMP1, BMP2, BMP3, BMP4, BMP5, BMP6, CSF2, CSF3, EGF, EGFR, FGF1, FGF2, FGF3, FGFR1, FGFR2, FLT1, GDF10, IGF1, IGF1R, IGF2, PDGFA, TGFB1, TGFB2, TGFB3, TGFBR1, TGFBR2, VEGFA, VEGFB, COL4A3, COL10A1, COL11A1, COL12A1, COL14A1, COL15A1, COL1A1, COL1A2, COL2A1, COL3A1, COL4A3, COL5A1, AHSG, COL4A3, SERPINH1, CTSK, MMP10, MMP2, MMP8, MMP9, and PHEX, CDH11, COL11A1, COL14A1, ICAM1, ITGB1, VCAM1, ITGA1, ITGA2, ITGA3, ITGAM, ITGB1, BGLAP, CD36, COL12A1, COL15A1, COL4A3, COL5A1, COMP, FN1, SCARB1, TNF, MSX1, NFKB1, RUNX2, SMAD1, SMAD2, SMAD3, SMAD4, SOX9, TNF, TWIST1, VDR, and combinations thereof.

51. The method of claim 52, wherein the osteogenic gene is selected from the group consisting of COX-2, OPN, BSP, Collagen I, Osteocalcin, and combinations thereof.

52. The method of claim 52, wherein the cell is comprised in a population of cells, and the cilium elongation modulator is contacted to the population of cells in an amount effective to increase osteogenesis.

53. The method of claim 1, wherein the cilium elongation modulator is selected from a dihydrofolate reductase antagonist, an inhibitor of DNA synthesis or RNA synthesis, a glycogen synthase kinase 3 inhibitor, a topoisomerase inhibitor, a nucleoside analogue, an HDAC inhibitor, a 5-HT modulator, an anthelmintic, an epidermal growth factor receptor kinase inhibitor, a histamine receptor modulator, a dopamine D1 receptor agonist, a checkpoint kinase 1 inhibitor, a microtubule stabilizer, a reactive oxygen species modulator, a sodium channel blocker, an adrenoreceptor modulator, an inosine-5′-monophosphate dehydrogenase inhibitor, a benzodiazepine inverse agonist, an opioid receptor modulator, and an antiseptic.

54. The method of claim 1, wherein the cilium elongation modulator is selected from (S)-(+)-Niguldipine hydrochloride (CAS No.: 113145-69-0), 10-hydroxycamptothecin (CAS No.: 19685-09-7), 110-Phenanthroline monohydrate (CAS No.: 5144-89-8), 2 4-dihydroxychalcone 4-glucoside (CAS No.: 25515-43-9), 2-Methoxyestradiol (CAS No.: 362-07-2), 5-BDBD (CAS No.: 768404-03-1), 5-fluorouracil (CAS No.: 51-21-8), 62-dimethoxyflavone, 6-aminonicotinamide (CAS No.: 329-89-5), AG 494 (CAS No.: 139087-53-9), AM 630 (CAS No.: 164178-33-0), Amethopterin (R,S) (CAS No.: 60388-53-6), aminopterin (CAS No.: 54-62-6), BAY 61-3606 hydrochloride hydrate (CAS No.: 732938-37-8), Benzethonium chloride (CAS No.: 121-54-0), betahistine hydrochloride (CAS No.: 15430-48-5), beta-toxicarol (CAS No.: 82-11-1), BIO (CAS No.: 667463-62-9), Bromhexine HCl (CAS No.: 3572-43-8), Bufexamac (CAS No.: 2438-72-4), BW 373U86 (CAS No.: 155836-50-3), CP 339818 hydrochloride (CAS No.: 478341-55-8), CPT 11 (CAS No.: 100286-90-6), CY 208-243 (CAS No.: 100999-26-6), cycloheximide (CAS No.: 66-81-9), Cyclosporin A (CAS No.: 59865-13-3), deacetoxy-7-oxogedunin (CAS No.: CAS No. 13072-74-7), DEGUELIN(−) (CAS No.: 522-17-8), dehydrocholic acid (CAS No.: 81-23-2), DEOXYGEDUNIN (CAS No.: 21963-95-1), deoxykhivorin, deoxysappanone b 73-dimethyl ether acetate (CAS No.: 113122-54-6), dihydrocelastryl diacetate, Diphenyleneiodonium chloride (CAS No.: 4673-26-1), Docetaxel (CAS No.: 114977-28-5), Dorzolamide HCl (CAS No.: 130693-82-2), Droxinostat (CAS No.: 99873-43-5), duartin dimethyl ether, estradiol cypionate (CAS No.: 313-06-4), Ethacridine lactate monohydrate (CAS No.: 1837-57-6), Etonitazenyl isothiocyanate (CAS No.: 85951-65-1), Floxuridine (CAS No.: 50-91-9), Ginkgolide B (CAS No.: 15291-77-7), GW 1929 (CAS No.: 196808-24-9), harmalol hydrochloride (CAS No.: 6028-07-5), hecogenin acetate (CAS No.: 915-35-5), homidium bromide (CAS No.: 1239-45-8), hycanthone (CAS No.: 3105-97-3), IC 261 (CAS No.: 186611-52-9), Indirubin-3′-oxime (CAS No.: 160807-49-8), Irinotecan (CAS No.: 97682-44-5), itraconazole (CAS No.: 84625-61-6), Kenpaullone (CAS No.: 142273-20-9), khivorin (CAS No.: 2524-38-1), levulinic acid 3-benzylidenyl-, Lonidamine (CAS No.: 50264-69-2), Loratidine (CAS No.: 79794-75-5), Merbromin (CAS No.: 129-16-8), mercaptopurine (CAS No.: 50-44-2), Methotrexate (CAS No.: 59-05-2), methoxyamine hydrochloride (CAS No.: 67-62-9), monensin sodium (CAS No.: 22373-78-0), monobenzone (CAS No.: 103-16-2), Mycophenolate mofetil (CAS No.: 128794-94-5), Oxfendazole (CAS No.: 53716-50-0), pararosaniline pamoate (CAS No.: 7460-07-3), PD 169316 (CAS No.: 152121-53-4)), PD-407824 (CAS No.: 622864-54-4), Pemetrexed (CAS No.: 137281-23-3), perillic acid (−) (CAS No.: 23635-14-5), PHA 767491 hydrochloride (CAS No.: 942425-68-5), PIM 1 Inhibitor 2 (CAS No.: 477845-12-8), Piperlongumine (CAS No.: 20069-09-4), Pralatrexate (CAS No.: 146464-95-1), pyrimethamine (CAS No.: 58-14-0), pyrvinium pamoate (CAS No.: 3546-41-6), quinidine gluconate (CAS No.: 56-54-2), quinine sulfate (CAS No.: 6119-70-6), reserpine (CAS No.: 50-55-5), Retinoic acid p-hydroxyanilide (CAS No.: 65646-68-6), Ro 19-4605, S 14506 hydrochloride (CAS No.: 286369-38-8), S(−)-Atenolol (CAS No.: 93379-54-5.), SANT-1 (CAS No.: 304909-07-7), SB 203580 (CAS No.: 152121-47-6), SB 206553 hydrochloride (CAS No.: 1197334-04-5), SB 228357 (CAS No.: 181629-93-6), SB 239063 (CAS No.: 193551-21-2), SB 408124 (CAS No.: 288150-92-5), SB 415286 (CAS No.: 264218-23-7), securinine (CAS No.: 5610-40-2), SKF 77434 hydrobromide (CAS No.: 300561-58-4), SKF 86002 dihydrochloride (CAS No.: 116339-68-5), Sorafenib (CAS No.: 284461-73-0), thioguanine (CAS No.: 154-42-7), Topotecan hydrochloride hydrate (CAS No.: 119413-54-6), Triamterene (CAS No.: 396-01-0), Trifluridine (CAS No.: 70-00-8), tulobuterol (CAS No.: 41570-61-0), Tyrphostin B44 (−) enantiomer (CAS No.: 133550-32-0), Vinblastine (CAS No.: 865-21-4), Vorinostat (CAS No.: 149647-78-9), VU 0155069 (CAS No.: 1130067-06-9), derivatives thereof, and combinations thereof.

55. The method of claim 20, wherein the cilium elongation modulator is selected from a dihydrofolate reductase antagonist, an inhibitor of DNA synthesis or RNA synthesis, a glycogen synthase kinase 3 inhibitor, a topoisomerase inhibitor, a nucleoside analogue, an HDAC inhibitor, a 5-HT modulator, an anthelmintic, an epidermal growth factor receptor kinase inhibitor, a histamine receptor modulator, a dopamine D1 receptor agonist, a checkpoint kinase 1 inhibitor, a microtubule stabilizer, a reactive oxygen species modulator, a sodium channel blocker, an adrenoreceptor modulator, an inosine-5′-monophosphate dehydrogenase inhibitor, a benzodiazepine inverse agonist, an opioid receptor modulator, and an antiseptic.

56. The method of claim 20, wherein the cilium elongation modulator is selected from (S)-(+)-Niguldipine hydrochloride (CAS No.: 113145-69-0), 10-hydroxycamptothecin (CAS No.: 19685-09-7), 110-Phenanthroline monohydrate (CAS No.: 5144-89-8), 2 4-dihydroxychalcone 4-glucoside (CAS No.: 25515-43-9), 2-Methoxyestradiol (CAS No.: 362-07-2), 5-BDBD (CAS No.: 768404-03-1), 5-fluorouracil (CAS No.: 51-21-8), 62-dimethoxyflavone, 6-aminonicotinamide (CAS No.: 329-89-5), AG 494 (CAS No.: 139087-53-9), AM 630 (CAS No.: 164178-33-0), Amethopterin (R,S) (CAS No.: 60388-53-6), aminopterin (CAS No.: 54-62-6), BAY 61-3606 hydrochloride hydrate (CAS No.: 732938-37-8), Benzethonium chloride (CAS No.: 121-54-0), betahistine hydrochloride (CAS No.: 15430-48-5), beta-toxicarol (CAS No.: 82-11-1), BIO (CAS No.: 667463-62-9), Bromhexine HCl (CAS No.: 3572-43-8), Bufexamac (CAS No.: 2438-72-4), BW 373U86 (CAS No.: 155836-50-3), CP 339818 hydrochloride (CAS No.: 478341-55-8), CPT 11 (CAS No.: 100286-90-6), CY 208-243 (CAS No.: 100999-26-6), cycloheximide (CAS No.: 66-81-9), Cyclosporin A (CAS No.: 59865-13-3), deacetoxy-7-oxogedunin (CAS No.: CAS No. 13072-74-7), DEGUELIN(−) (CAS No.: 522-17-8), dehydrocholic acid (CAS No.: 81-23-2), DEOXYGEDUNIN (CAS No.: 21963-95-1), deoxykhivorin, deoxysappanone b 73-dimethyl ether acetate (CAS No.: 113122-54-6), dihydrocelastryl diacetate, Diphenyleneiodonium chloride (CAS No.: 4673-26-1), Docetaxel (CAS No.: 114977-28-5), Dorzolamide HCl (CAS No.: 130693-82-2), Droxinostat (CAS No.: 99873-43-5), duartin dimethyl ether, estradiol cypionate (CAS No.: 313-06-4), Ethacridine lactate monohydrate (CAS No.: 1837-57-6), Etonitazenyl isothiocyanate (CAS No.: 85951-65-1), Floxuridine (CAS No.: 50-91-9), Ginkgolide B (CAS No.: 15291-77-7), GW 1929 (CAS No.: 196808-24-9), harmalol hydrochloride (CAS No.: 6028-07-5), hecogenin acetate (CAS No.: 915-35-5), homidium bromide (CAS No.: 1239-45-8), hycanthone (CAS No.: 3105-97-3), IC 261 (CAS No.: 186611-52-9), Indirubin-3′-oxime (CAS No.: 160807-49-8), Irinotecan (CAS No.: 97682-44-5), itraconazole (CAS No.: 84625-61-6), Kenpaullone (CAS No.: 142273-20-9), khivorin (CAS No.: 2524-38-1), levulinic acid 3-benzylidenyl-, Lonidamine (CAS No.: 50264-69-2), Loratidine (CAS No.: 79794-75-5), Merbromin (CAS No.: 129-16-8), mercaptopurine (CAS No.: 50-44-2), Methotrexate (CAS No.: 59-05-2), methoxyamine hydrochloride (CAS No.: 67-62-9), monensin sodium (CAS No.: 22373-78-0), monobenzone (CAS No.: 103-16-2), Mycophenolate mofetil (CAS No.: 128794-94-5), Oxfendazole (CAS No.: 53716-50-0), pararosaniline pamoate (CAS No.: 7460-07-3), PD 169316 (CAS No.: 152121-53-4)), PD-407824 (CAS No.: 622864-54-4), Pemetrexed (CAS No.: 137281-23-3), perillic acid (−) (CAS No.: 23635-14-5), PHA 767491 hydrochloride (CAS No.: 942425-68-5), PIM 1 Inhibitor 2 (CAS No.: 477845-12-8), Piperlongumine (CAS No.: 20069-09-4), Pralatrexate (CAS No.: 146464-95-1), pyrimethamine (CAS No.: 58-14-0), pyrvinium pamoate (CAS No.: 3546-41-6), quinidine gluconate (CAS No.: 56-54-2), quinine sulfate (CAS No.: 6119-70-6), reserpine (CAS No.: 50-55-5), Retinoic acid p-hydroxyanilide (CAS No.: 65646-68-6), Ro 19-4605, S 14506 hydrochloride (CAS No.: 286369-38-8), S(−)-Atenolol (CAS No.: 93379-54-5.), SANT-1 (CAS No.: 304909-07-7), SB 203580 (CAS No.: 152121-47-6), SB 206553 hydrochloride (CAS No.: 1197334-04-5), SB 228357 (CAS No.: 181629-93-6), SB 239063 (CAS No.: 193551-21-2), SB 408124 (CAS No.: 288150-92-5), SB 415286 (CAS No.: 264218-23-7), securinine (CAS No.: 5610-40-2), SKF 77434 hydrobromide (CAS No.: 300561-58-4), SKF 86002 dihydrochloride (CAS No.: 116339-68-5), Sorafenib (CAS No.: 284461-73-0), thioguanine (CAS No.: 154-42-7), Topotecan hydrochloride hydrate (CAS No.: 119413-54-6), Triamterene (CAS No.: 396-01-0), Trifluridine (CAS No.: 70-00-8), tulobuterol (CAS No.: 41570-61-0), Tyrphostin B44 (−) enantiomer (CAS No.: 133550-32-0), Vinblastine (CAS No.: 865-21-4), Vorinostat (CAS No.: 149647-78-9), VU 0155069 (CAS No.: 1130067-06-9), derivatives thereof, and combinations thereof.

57. The method of claim 37, wherein the cilium elongation modulator is selected from a dihydrofolate reductase antagonist, an inhibitor of DNA synthesis or RNA synthesis, a glycogen synthase kinase 3 inhibitor, a topoisomerase inhibitor, a nucleoside analogue, an HDAC inhibitor, a 5-HT modulator, an anthelmintic, an epidermal growth factor receptor kinase inhibitor, a histamine receptor modulator, a dopamine D1 receptor agonist, a checkpoint kinase 1 inhibitor, a microtubule stabilizer, a reactive oxygen species modulator, a sodium channel blocker, an adrenoreceptor modulator, an inosine-5′-monophosphate dehydrogenase inhibitor, a benzodiazepine inverse agonist, an opioid receptor modulator, and an antiseptic.

58. The method of claim 37, wherein the cilium elongation modulator is selected from (S)-(+)-Niguldipine hydrochloride (CAS No.: 113145-69-0), 10-hydroxycamptothecin (CAS No.: 19685-09-7), 110-Phenanthroline monohydrate (CAS No.: 5144-89-8), 2 4-dihydroxychalcone 4-glucoside (CAS No.: 25515-43-9), 2-Methoxyestradiol (CAS No.: 362-07-2), 5-BDBD (CAS No.: 768404-03-1), 5-fluorouracil (CAS No.: 51-21-8), 62-dimethoxyflavone, 6-aminonicotinamide (CAS No.: 329-89-5), AG 494 (CAS No.: 139087-53-9), AM 630 (CAS No.: 164178-33-0), Amethopterin (R,S) (CAS No.: 60388-53-6), aminopterin (CAS No.: 54-62-6), BAY 61-3606 hydrochloride hydrate (CAS No.: 732938-37-8), Benzethonium chloride (CAS No.: 121-54-0), betahistine hydrochloride (CAS No.: 15430-48-5), beta-toxicarol (CAS No.: 82-11-1), BIO (CAS No.: 667463-62-9), Bromhexine HCl (CAS No.: 3572-43-8), Bufexamac (CAS No.: 2438-72-4), BW 373U86 (CAS No.: 155836-50-3), CP 339818 hydrochloride (CAS No.: 478341-55-8), CPT 11 (CAS No.: 100286-90-6), CY 208-243 (CAS No.: 100999-26-6), cycloheximide (CAS No.: 66-81-9), Cyclosporin A (CAS No.: 59865-13-3), deacetoxy-7-oxogedunin (CAS No.: CAS No. 13072-74-7), DEGUELIN(−) (CAS No.: 522-17-8), dehydrocholic acid (CAS No.: 81-23-2), DEOXYGEDUNIN (CAS No.: 21963-95-1), deoxykhivorin, deoxysappanone b 73-dimethyl ether acetate (CAS No.: 113122-54-6), dihydrocelastryl diacetate, Diphenyleneiodonium chloride (CAS No.: 4673-26-1), Docetaxel (CAS No.: 114977-28-5), Dorzolamide HCl (CAS No.: 130693-82-2), Droxinostat (CAS No.: 99873-43-5), duartin dimethyl ether, estradiol cypionate (CAS No.: 313-06-4), Ethacridine lactate monohydrate (CAS No.: 1837-57-6), Etonitazenyl isothiocyanate (CAS No.: 85951-65-1), Floxuridine (CAS No.: 50-91-9), Ginkgolide B (CAS No.: 15291-77-7), GW 1929 (CAS No.: 196808-24-9), harmalol hydrochloride (CAS No.: 6028-07-5), hecogenin acetate (CAS No.: 915-35-5), homidium bromide (CAS No.: 1239-45-8), hycanthone (CAS No.: 3105-97-3), IC 261 (CAS No.: 186611-52-9), Indirubin-3′-oxime (CAS No.: 160807-49-8), Irinotecan (CAS No.: 97682-44-5), itraconazole (CAS No.: 84625-61-6), Kenpaullone (CAS No.: 142273-20-9), khivorin (CAS No.: 2524-38-1), levulinic acid 3-benzylidenyl-, Lonidamine (CAS No.: 50264-69-2), Loratidine (CAS No.: 79794-75-5), Merbromin (CAS No.: 129-16-8), mercaptopurine (CAS No.: 50-44-2), Methotrexate (CAS No.: 59-05-2), methoxyamine hydrochloride (CAS No.: 67-62-9), monensin sodium (CAS No.: 22373-78-0), monobenzone (CAS No.: 103-16-2), Mycophenolate mofetil (CAS No.: 128794-94-5), Oxfendazole (CAS No.: 53716-50-0), pararosaniline pamoate (CAS No.: 7460-07-3), PD 169316 (CAS No.: 152121-53-4)), PD-407824 (CAS No.: 622864-54-4), Pemetrexed (CAS No.: 137281-23-3), perillic acid (−) (CAS No.: 23635-14-5), PHA 767491 hydrochloride (CAS No.: 942425-68-5), PIM 1 Inhibitor 2 (CAS No.: 477845-12-8), Piperlongumine (CAS No.: 20069-09-4), Pralatrexate (CAS No.: 146464-95-1), pyrimethamine (CAS No.: 58-14-0), pyrvinium pamoate (CAS No.: 3546-41-6), quinidine gluconate (CAS No.: 56-54-2), quinine sulfate (CAS No.: 6119-70-6), reserpine (CAS No.: 50-55-5), Retinoic acid p-hydroxyanilide (CAS No.: 65646-68-6), Ro 19-4605, S 14506 hydrochloride (CAS No.: 286369-38-8), S(−)-Atenolol (CAS No.: 93379-54-5.), SANT-1 (CAS No.: 304909-07-7), SB 203580 (CAS No.: 152121-47-6), SB 206553 hydrochloride (CAS No.: 1197334-04-5), SB 228357 (CAS No.: 181629-93-6), SB 239063 (CAS No.: 193551-21-2), SB 408124 (CAS No.: 288150-92-5), SB 415286 (CAS No.: 264218-23-7), securinine (CAS No.: 5610-40-2), SKF 77434 hydrobromide (CAS No.: 300561-58-4), SKF 86002 dihydrochloride (CAS No.: 116339-68-5), Sorafenib (CAS No.: 284461-73-0), thioguanine (CAS No.: 154-42-7), Topotecan hydrochloride hydrate (CAS No.: 119413-54-6), Triamterene (CAS No.: 396-01-0), Trifluridine (CAS No.: 70-00-8), tulobuterol (CAS No.: 41570-61-0), Tyrphostin B44 (−) enantiomer (CAS No.: 133550-32-0), Vinblastine (CAS No.: 865-21-4), Vorinostat (CAS No.: 149647-78-9), VU 0155069 (CAS No.: 1130067-06-9), derivatives thereof, and combinations thereof.


SEQUENCE LISTING (2 sequences)

SEQ ID NO: 1 - DNA, 25 bp, Artificial Sequence
  siRNA
ccagaaacagatgaggacgaccttt

SEQ ID NO: 2 - DNA, 25 bp, Artificial Sequence
  siRNA
cctgccacctacaacagctcaatta